SEQUENCE LISTING (694 sequences)

SEQ ID NO: 1 - DNA, 20 bp, Artificial Sequence
  Description of Artificial Sequence; Note= Synthetic Construct
aattggcaccagagaaatgg

SEQ ID NO: 2 - DNA, 24 bp, Artificial Sequence
  Description of Artificial Sequence; Note= Synthetic Construct
tcatgtgtttagttctatcgcaaa

SEQ ID NO: 3 - DNA, 20 bp, Artificial Sequence
  Description of Artificial Sequence; Note= Synthetic Construct
tcatttctccatggttgggt

SEQ ID NO: 4 - DNA, 21 bp, Artificial Sequence
  Description of Artificial Sequence; Note= Synthetic Construct
tgacatcccaataaacctcca

SEQ ID NO: 5 - DNA, 20 bp, Artificial Sequence
  Description of Artificial Sequence; Note= Synthetic Construct
gctctcacagggttgtttca

SEQ ID NO: 6 - DNA, 22 bp, Artificial Sequence
  Description of Artificial Sequence; Note= Synthetic Construct
gaagggcaaagataagagacga

SEQ ID NO: 7 - DNA, 20 bp, Artificial Sequence
  Description of Artificial Sequence; Note= Synthetic Construct
gggaaccaaagtgattgagg

SEQ ID NO: 8 - DNA, 21 bp, Artificial Sequence
  Description of Artificial Sequence; Note= Synthetic Construct
tgcttggagacagcgtttaat

SEQ ID NO: 9 - DNA, 21 bp, Artificial Sequence
  Description of Artificial Sequence; Note= Synthetic Construct
caggagacacagagatttgcc

SEQ ID NO: 10 - DNA, 20 bp, Artificial Sequence
  Description of Artificial Sequence; Note= Synthetic Construct
tcggaagaccctatgctcag

SEQ ID NO: 11 - DNA, 22 bp, Artificial Sequence
  Description of Artificial Sequence; Note= Synthetic Construct
gcttgcgttaaatgatggtatg

SEQ ID NO: 12 - DNA, 20 bp, Artificial Sequence
  Description of Artificial Sequence; Note= Synthetic Construct
ttgatttgctgttccagtgc

SEQ ID NO: 13 - DNA, 25 bp, Artificial Sequence
  Description of Artificial Sequence; Note= Synthetic Construct
gcgtagattatttgtcatcttcagg

SEQ ID NO: 14 - DNA, 22 bp, Artificial Sequence
  Description of Artificial Sequence; Note= Synthetic Construct
tgagtaaccatccaacagagga

SEQ ID NO: 15 - DNA, 20 bp, Artificial Sequence
  Description of Artificial Sequence; Note= Synthetic Construct
ttatcccatgcaccacaatg

SEQ ID NO: 16 - DNA, 20 bp, Artificial Sequence
  Description of Artificial Sequence; Note= Synthetic Construct
caagccaatgtcatggaaga

SEQ ID NO: 17 - DNA, 20 bp, Artificial Sequence
  Description of Artificial Sequence; Note= Synthetic Construct
ctgctgaatgtgtggagagc

SEQ ID NO: 18 - DNA, 20 bp, Artificial Sequence
  Description of Artificial Sequence; Note= Synthetic Construct
acatttcattcccacgctgt

SEQ ID NO: 19 - DNA, 20 bp, Artificial Sequence
  Description of Artificial Sequence; Note= Synthetic Construct
ggccttctggaaataaaggc

SEQ ID NO: 20 - DNA, 20 bp, Artificial Sequence
  Description of Artificial Sequence; Note= Synthetic Construct
aaacttgtggcccatttaga

SEQ ID NO: 21 - DNA, 20 bp, Artificial Sequence
  Description of Artificial Sequence; Note= Synthetic Construct
gccaacaggaatacgaaagc

SEQ ID NO: 22 - DNA, 20 bp, Artificial Sequence
  Description of Artificial Sequence; Note= Synthetic Construct
ttcaaatccacagttggcac

SEQ ID NO: 23 - DNA, 23 bp, Artificial Sequence
  Description of Artificial Sequence; Note= Synthetic Construct
tgcagaatcactcctatatggtc

SEQ ID NO: 24 - DNA, 20 bp, Artificial Sequence
  Description of Artificial Sequence; Note= Synthetic Construct
cataccctgcgttgttctca

SEQ ID NO: 25 - DNA, 22 bp, Artificial Sequence
  Description of Artificial Sequence; Note= Synthetic Construct
ggagaacatcctgctgtacctt

SEQ ID NO: 26 - DNA, 20 bp, Artificial Sequence
  Description of Artificial Sequence; Note= Synthetic Construct
tagcaagggctttctctcca

SEQ ID NO: 27 - DNA, 22 bp, Artificial Sequence
  Description of Artificial Sequence; Note= Synthetic Construct
ttcctttgcatagaaagcatca

SEQ ID NO: 28 - DNA, 21 bp, Artificial Sequence
  Description of Artificial Sequence; Note= Synthetic Construct
aaccgtcgcttgtaactctca

SEQ ID NO: 29 - DNA, 21 bp, Artificial Sequence
  Description of Artificial Sequence; Note= Synthetic Construct
ccaaatggtaggcaattctca

SEQ ID NO: 30 - DNA, 20 bp, Artificial Sequence
  Description of Artificial Sequence; Note= Synthetic Construct
aatgtcaggataaccgtcgc

SEQ ID NO: 31 - DNA, 20 bp, Artificial Sequence
  Description of Artificial Sequence; Note= Synthetic Construct
cagcatttcagaatggcaag

SEQ ID NO: 32 - DNA, 21 bp, Artificial Sequence
  Description of Artificial Sequence; Note= Synthetic Construct
tgaaatcagcagtctatggca

SEQ ID NO: 33 - DNA, 22 bp, Artificial Sequence
  Description of Artificial Sequence; Note= Synthetic Construct
tgtctccagtgatgaatatggg

SEQ ID NO: 34 - DNA, 20 bp, Artificial Sequence
  Description of Artificial Sequence; Note= Synthetic Construct
tgcgcagactgagacatcat

SEQ ID NO: 35 - DNA, 20 bp, Artificial Sequence
  Description of Artificial Sequence; Note= Synthetic Construct
aagctctgacatgcaagcac

SEQ ID NO: 36 - DNA, 20 bp, Artificial Sequence
  Description of Artificial Sequence; Note= Synthetic Construct
actgagaaaggctggacacc

SEQ ID NO: 37 - DNA, 22 bp, Artificial Sequence
  Description of Artificial Sequence; Note= Synthetic Construct
ttgtcttccttggaaataggag

SEQ ID NO: 38 - DNA, 23 bp, Artificial Sequence
  Description of Artificial Sequence; Note= Synthetic Construct
tttggaaatagcattatccctga

SEQ ID NO: 39 - DNA, 23 bp, Artificial Sequence
  Description of Artificial Sequence; Note= Synthetic Construct
atttaaactaatttccaagccca

SEQ ID NO: 40 - DNA, 20 bp, Artificial Sequence
  Description of Artificial Sequence; Note= Synthetic Construct
acactatccggtgtggttcc

SEQ ID NO: 41 - DNA, 20 bp, Artificial Sequence
  Description of Artificial Sequence; Note= Synthetic Construct
gcctgtttggtcaggacaag

SEQ ID NO: 42 - DNA, 20 bp, Artificial Sequence
  Description of Artificial Sequence; Note= Synthetic Construct
gctgagtttcagttgccaca

SEQ ID NO: 43 - DNA, 21 bp, Artificial Sequence
  Description of Artificial Sequence; Note= Synthetic Construct
ttgcaattgggattaacaatg

SEQ ID NO: 44 - DNA, 20 bp, Artificial Sequence
  Description of Artificial Sequence; Note= Synthetic Construct
cccaccagtttgagaatgtg

SEQ ID NO: 45 - DNA, 21 bp, Artificial Sequence
  Description of Artificial Sequence; Note= Synthetic Construct
atccttgaatcccaccataat

SEQ ID NO: 46 - DNA, 26 bp, Artificial Sequence
  Description of Artificial Sequence; Note= Synthetic Construct
cagcagaaatgaaaggtaatatagga

SEQ ID NO: 47 - DNA, 20 bp, Artificial Sequence
  Description of Artificial Sequence; Note= Synthetic Construct
gggaaagaatcatgggtgag

SEQ ID NO: 48 - DNA, 20 bp, Artificial Sequence
  Description of Artificial Sequence; Note= Synthetic Construct
cttcctgctgcatgacaatg

SEQ ID NO: 49 - DNA, 20 bp, Artificial Sequence
  Description of Artificial Sequence; Note= Synthetic Construct
cattgtcatgcagcaggaag

SEQ ID NO: 50 - DNA, 20 bp, Artificial Sequence
  Description of Artificial Sequence; Note= Synthetic Construct
atgtgtcgaagaggccaaac

SEQ ID NO: 51 - DNA, 20 bp, Artificial Sequence
  Description of Artificial Sequence; Note= Synthetic Construct
tgaattatcatcatcgggca

SEQ ID NO: 52 - DNA, 21 bp, Artificial Sequence
  Description of Artificial Sequence; Note= Synthetic Construct
ccttgtcacaatccttgaacc

SEQ ID NO: 53 - DNA, 21 bp, Artificial Sequence
  Description of Artificial Sequence; Note= Synthetic Construct
cacaaatccatacctccatgc

SEQ ID NO: 54 - DNA, 20 bp, Artificial Sequence
  Description of Artificial Sequence; Note= Synthetic Construct
ttgaggcacctgctttcttt

SEQ ID NO: 55 - DNA, 24 bp, Artificial Sequence
  Description of Artificial Sequence; Note= Synthetic Construct
tccatattcacgatgatgtttacc

SEQ ID NO: 56 - DNA, 25 bp, Artificial Sequence
  Description of Artificial Sequence; Note= Synthetic Construct
gagcttgaatgattaaatgtcagaa

SEQ ID NO: 57 - DNA, 20 bp, Artificial Sequence
  Description of Artificial Sequence; Note= Synthetic Construct
gcgagtaggcactctctgct

SEQ ID NO: 58 - DNA, 23 bp, Artificial Sequence
  Description of Artificial Sequence; Note= Synthetic Construct
tcttgcacattctaggaaatcag

SEQ ID NO: 59 - DNA, 20 bp, Artificial Sequence
  Description of Artificial Sequence; Note= Synthetic Construct
gatcatgcaaagctggttga

SEQ ID NO: 60 - DNA, 20 bp, Artificial Sequence
  Description of Artificial Sequence; Note= Synthetic Construct
tgctttccaacaatgccata

SEQ ID NO: 61 - DNA, 19 bp, Artificial Sequence
  Description of Artificial Sequence; Note= Synthetic Construct
agtatctgccggaagccat

SEQ ID NO: 62 - DNA, 22 bp, Artificial Sequence
  Description of Artificial Sequence; Note= Synthetic Construct
gcaagtgcatcttcacttcatc

SEQ ID NO: 63 - DNA, 21 bp, Artificial Sequence
  Description of Artificial Sequence; Note= Synthetic Construct
catggtagaggtggttgagga

SEQ ID NO: 64 - DNA, 20 bp, Artificial Sequence
  Description of Artificial Sequence; Note= Synthetic Construct
attcggtgttgtcttgaggc

SEQ ID NO: 65 - DNA, 24 bp, Artificial Sequence
  Description of Artificial Sequence; Note= Synthetic Construct
ttcatccaaatttatggctagaat

SEQ ID NO: 66 - DNA, 21 bp, Artificial Sequence
  Description of Artificial Sequence; Note= Synthetic Construct
agttgagcgaagtgagatgga

SEQ ID NO: 67 - DNA, 20 bp, Artificial Sequence
  Description of Artificial Sequence; Note= Synthetic Construct
ctgagaacaggagcacagga

SEQ ID NO: 68 - DNA, 20 bp, Artificial Sequence
  Description of Artificial Sequence; Note= Synthetic Construct
gctgtgtcatttggtgatgg

SEQ ID NO: 69 - DNA, 21 bp, Artificial Sequence
  Description of Artificial Sequence; Note= Synthetic Construct
gggcagtttcttatttgtgga

SEQ ID NO: 70 - DNA, 20 bp, Artificial Sequence
  Description of Artificial Sequence; Note= Synthetic Construct
taccaccattgacaaaggca

SEQ ID NO: 71 - DNA, 20 bp, Artificial Sequence
  Description of Artificial Sequence; Note= Synthetic Construct
ccatacagaaagccgtttca

SEQ ID NO: 72 - DNA, 21 bp, Artificial Sequence
  Description of Artificial Sequence; Note= Synthetic Construct
gacagggcatcctaacagtca

SEQ ID NO: 73 - DNA, 20 bp, Artificial Sequence
  Description of Artificial Sequence; Note= Synthetic Construct
acttcaacctctgtgacccg

SEQ ID NO: 74 - DNA, 20 bp, Artificial Sequence
  Description of Artificial Sequence; Note= Synthetic Construct
accctagaccgtgcagaaga

SEQ ID NO: 75 - DNA, 20 bp, Artificial Sequence
  Description of Artificial Sequence; Note= Synthetic Construct
tgcatcaccaaccaaactgt

SEQ ID NO: 76 - DNA, 20 bp, Artificial Sequence
  Description of Artificial Sequence; Note= Synthetic Construct
cagaggtgatggcagtgaaa

SEQ ID NO: 77 - DNA, 21 bp, Artificial Sequence
  Description of Artificial Sequence; Note= Synthetic Construct
ggtttcagaaatgaagcagga

SEQ ID NO: 78 - DNA, 20 bp, Artificial Sequence
  Description of Artificial Sequence; Note= Synthetic Construct
tcctgcacaaaccagatgag

SEQ ID NO: 79 - DNA, 20 bp, Artificial Sequence
  Description of Artificial Sequence; Note= Synthetic Construct
agccttggaaggagaagcat

SEQ ID NO: 80 - DNA, 20 bp, Artificial Sequence
  Description of Artificial Sequence; Note= Synthetic Construct
attcctctggtgtcttggga

SEQ ID NO: 81 - DNA, 21 bp, Artificial Sequence
  Description of Artificial Sequence; Note= Synthetic Construct
agcccattcatttcatcagag

SEQ ID NO: 82 - DNA, 20 bp, Artificial Sequence
  Description of Artificial Sequence; Note= Synthetic Construct
atggcttatgcaggttgaca

SEQ ID NO: 83 - DNA, 20 bp, Artificial Sequence
  Description of Artificial Sequence; Note= Synthetic Construct
gaaatttaaatgccggttgc

SEQ ID NO: 84 - DNA, 20 bp, Artificial Sequence
  Description of Artificial Sequence; Note= Synthetic Construct
gcttccaggaaaccatttga

SEQ ID NO: 85 - DNA, 20 bp, Artificial Sequence
  Description of Artificial Sequence; Note= Synthetic Construct
caccatttgctacctttggg

SEQ ID NO: 86 - DNA, 22 bp, Artificial Sequence
  Description of Artificial Sequence; Note= Synthetic Construct
ttcagctcatttgtctgaattg

SEQ ID NO: 87 - DNA, 20 bp, Artificial Sequence
  Description of Artificial Sequence; Note= Synthetic Construct
ggattaaagaaggcatcgca

SEQ ID NO: 88 - DNA, 20 bp, Artificial Sequence
  Description of Artificial Sequence; Note= Synthetic Construct
ggttccaacataaagccgaa

SEQ ID NO: 89 - DNA, 20 bp, Artificial Sequence
  Description of Artificial Sequence; Note= Synthetic Construct
atcttgatgggatgctcctg

SEQ ID NO: 90 - DNA, 20 bp, Artificial Sequence
  Description of Artificial Sequence; Note= Synthetic Construct
catttggctttctgtgcctt

SEQ ID NO: 91 - DNA, 27 bp, Artificial Sequence
  Description of Artificial Sequence; Note= Synthetic Construct
cagatataatgacataatgttgttaga

SEQ ID NO: 92 - DNA, 21 bp, Artificial Sequence
  Description of Artificial Sequence; Note= Synthetic Construct
gcaatccagatcttccctaag

SEQ ID NO: 93 - DNA, 20 bp, Artificial Sequence
  Description of Artificial Sequence; Note= Synthetic Construct
gtcttgggaaagggcataca

SEQ ID NO: 94 - DNA, 23 bp, Artificial Sequence
  Description of Artificial Sequence; Note= Synthetic Construct
atagtatgcaaggtggaaagatg

SEQ ID NO: 95 - DNA, 26 bp, Artificial Sequence
  Description of Artificial Sequence; Note= Synthetic Construct
cctataatcattctgttacagtctac

SEQ ID NO: 96 - DNA, 23 bp, Artificial Sequence
  Description of Artificial Sequence; Note= Synthetic Construct
gaagcctgtcagtttacaagaac

SEQ ID NO: 97 - DNA, 23 bp, Artificial Sequence
  Description of Artificial Sequence; Note= Synthetic Construct
tgctttaagtgtttaccctttgg

SEQ ID NO: 98 - DNA, 20 bp, Artificial Sequence
  Description of Artificial Sequence; Note= Synthetic Construct
ctgacctggctttccatctc

SEQ ID NO: 99 - DNA, 22 bp, Artificial Sequence
  Description of Artificial Sequence; Note= Synthetic Construct
gctagttgctgagagggaactg

SEQ ID NO: 100 - DNA, 20 bp, Artificial Sequence
  Description of Artificial Sequence; Note= Synthetic Construct
aagccagcattaacattgcc

SEQ ID NO: 101 - DNA, 20 bp, Artificial Sequence
  Description of Artificial Sequence; Note= Synthetic Construct
ttcattggctttgatttccc

SEQ ID NO: 102 - DNA, 20 bp, Artificial Sequence
  Description of Artificial Sequence; Note= Synthetic Construct
gaaggcaaattggcacagac

SEQ ID NO: 103 - DNA, 20 bp, Artificial Sequence
  Description of Artificial Sequence; Note= Synthetic Construct
gatgctctccaaacttgcct

SEQ ID NO: 104 - DNA, 20 bp, Artificial Sequence
  Description of Artificial Sequence; Note= Synthetic Construct
aagttcctgcccaccctact

SEQ ID NO: 105 - DNA, 25 bp, Artificial Sequence
  Description of Artificial Sequence; Note= Synthetic Construct
cagaaactaatatttgccatcaaaa

SEQ ID NO: 106 - DNA, 20 bp, Artificial Sequence
  Description of Artificial Sequence; Note= Synthetic Construct
gagaagaatgagctgggctg

SEQ ID NO: 107 - DNA, 20 bp, Artificial Sequence
  Description of Artificial Sequence; Note= Synthetic Construct
aagcctcctctctgcacttg

SEQ ID NO: 108 - DNA, 20 bp, Artificial Sequence
  Description of Artificial Sequence; Note= Synthetic Construct
cgagtcatattgccctccac

SEQ ID NO: 109 - DNA, 20 bp, Artificial Sequence
  Description of Artificial Sequence; Note= Synthetic Construct
agcagcatcaaagacaagca

SEQ ID NO: 110 - DNA, 20 bp, Artificial Sequence
  Description of Artificial Sequence; Note= Synthetic Construct
cgacaaattcagccatctca

SEQ ID NO: 111 - DNA, 20 bp, Artificial Sequence
  Description of Artificial Sequence; Note= Synthetic Construct
ggccaagtgcaatcttgttt

SEQ ID NO: 112 - DNA, 20 bp, Artificial Sequence
  Description of Artificial Sequence; Note= Synthetic Construct
ttcctccacggaactattgc

SEQ ID NO: 113 - DNA, 20 bp, Artificial Sequence
  Description of Artificial Sequence; Note= Synthetic Construct
ggctgcctagggtgtagaaa

SEQ ID NO: 114 - DNA, 21 bp, Artificial Sequence
  Description of Artificial Sequence; Note= Synthetic Construct
ttgattgcatgttgaaatgac

SEQ ID NO: 115 - DNA, 20 bp, Artificial Sequence
  Description of Artificial Sequence; Note= Synthetic Construct
tccgcaattcctacatccat

SEQ ID NO: 116 - DNA, 20 bp, Artificial Sequence
  Description of Artificial Sequence; Note= Synthetic Construct
gctttcgtagaagccgagtg

SEQ ID NO: 117 - DNA, 20 bp, Artificial Sequence
  Description of Artificial Sequence; Note= Synthetic Construct
accaggagcccagaggtaat

SEQ ID NO: 118 - DNA, 20 bp, Artificial Sequence
  Description of Artificial Sequence; Note= Synthetic Construct
agggcaacacattaacagcc

SEQ ID NO: 119 - DNA, 26 bp, Artificial Sequence
  Description of Artificial Sequence; Note= Synthetic Construct
ccattgttataatactaccacaagag

SEQ ID NO: 120 - DNA, 20 bp, Artificial Sequence
  Description of Artificial Sequence; Note= Synthetic Construct
gtggcaattcacatcttcca

SEQ ID NO: 121 - DNA, 20 bp, Artificial Sequence
  Description of Artificial Sequence; Note= Synthetic Construct
ccaaatttaagccttgcctg

SEQ ID NO: 122 - DNA, 23 bp, Artificial Sequence
  Description of Artificial Sequence; Note= Synthetic Construct
tgaactgaactgataggcagaaa

SEQ ID NO: 123 - DNA, 22 bp, Artificial Sequence
  Description of Artificial Sequence; Note= Synthetic Construct
gcaaagatcattcatttgacca

SEQ ID NO: 124 - DNA, 20 bp, Artificial Sequence
  Description of Artificial Sequence; Note= Synthetic Construct
agtcgaggactgctgctttc

SEQ ID NO: 125 - DNA, 20 bp, Artificial Sequence
  Description of Artificial Sequence; Note= Synthetic Construct
gcttcattcaggcccaagta

SEQ ID NO: 126 - DNA, 21 bp, Artificial Sequence
  Description of Artificial Sequence; Note= Synthetic Construct
gacaaaccagacatctggaca

SEQ ID NO: 127 - DNA, 21 bp, Artificial Sequence
  Description of Artificial Sequence; Note= Synthetic Construct
agtttatgggcttgtggatga

SEQ ID NO: 128 - DNA, 20 bp, Artificial Sequence
  Description of Artificial Sequence; Note= Synthetic Construct
gcacacagaccctcagacaa

SEQ ID NO: 129 - DNA, 20 bp, Artificial Sequence
  Description of Artificial Sequence; Note= Synthetic Construct
gcagagagatgctgaggtga

SEQ ID NO: 130 - DNA, 20 bp, Artificial Sequence
  Description of Artificial Sequence; Note= Synthetic Construct
atctcccttgtgtgcaatcc

SEQ ID NO: 131 - DNA, 22 bp, Artificial Sequence
  Description of Artificial Sequence; Note= Synthetic Construct
tgtgttatgtggcctgaagtaa

SEQ ID NO: 132 - DNA, 20 bp, Artificial Sequence
  Description of Artificial Sequence; Note= Synthetic Construct
caactgcagcctttcacaat

SEQ ID NO: 133 - DNA, 21 bp, Artificial Sequence
  Description of Artificial Sequence; Note= Synthetic Construct
cctgtgggaacacatacatga

SEQ ID NO: 134 - DNA, 20 bp, Artificial Sequence
  Description of Artificial Sequence; Note= Synthetic Construct
gcaatgggacaggataggaa

SEQ ID NO: 135 - DNA, 20 bp, Artificial Sequence
  Description of Artificial Sequence; Note= Synthetic Construct
cagacagaatcaacagggca

SEQ ID NO: 136 - DNA, 21 bp, Artificial Sequence
  Description of Artificial Sequence; Note= Synthetic Construct
tggtgcaaagtgaatgagaga

SEQ ID NO: 137 - DNA, 25 bp, Artificial Sequence
  Description of Artificial Sequence; Note= Synthetic Construct
tttgaagatgaatcgtatcagtcaa

SEQ ID NO: 138 - DNA, 20 bp, Artificial Sequence
  Description of Artificial Sequence; Note= Synthetic Construct
ctacatccttgccatttccc

SEQ ID NO: 139 - DNA, 20 bp, Artificial Sequence
  Description of Artificial Sequence; Note= Synthetic Construct
tcctcccagatatttgcctg

SEQ ID NO: 140 - DNA, 20 bp, Artificial Sequence
  Description of Artificial Sequence; Note= Synthetic Construct
ggaaagcaatagccaaacca

SEQ ID NO: 141 - DNA, 20 bp, Artificial Sequence
  Description of Artificial Sequence; Note= Synthetic Construct
ctctcagctgaacaccctcc

SEQ ID NO: 142 - DNA, 20 bp, Artificial Sequence
  Description of Artificial Sequence; Note= Synthetic Construct
cctgataaacagtccgcaca

SEQ ID NO: 143 - DNA, 20 bp, Artificial Sequence
  Description of Artificial Sequence; Note= Synthetic Construct
cttgctgctgaattggaaga

SEQ ID NO: 144 - DNA, 20 bp, Artificial Sequence
  Description of Artificial Sequence; Note= Synthetic Construct
ttgacaaaggatggatgcac

SEQ ID NO: 145 - DNA, 20 bp, Artificial Sequence
  Description of Artificial Sequence; Note= Synthetic Construct
acttgccctctaacgtgcat

SEQ ID NO: 146 - DNA, 20 bp, Artificial Sequence
  Description of Artificial Sequence; Note= Synthetic Construct
ggcaggtttggtcaaagatt

SEQ ID NO: 147 - DNA, 20 bp, Artificial Sequence
  Description of Artificial Sequence; Note= Synthetic Construct
cttggtggccaaagcattat

SEQ ID NO: 148 - DNA, 20 bp, Artificial Sequence
  Description of Artificial Sequence; Note= Synthetic Construct
gggctcaaccaaagagatga

SEQ ID NO: 149 - DNA, 20 bp, Artificial Sequence
  Description of Artificial Sequence; Note= Synthetic Construct
tggcattatctccttgaggg

SEQ ID NO: 150 - DNA, 21 bp, Artificial Sequence
  Description of Artificial Sequence; Note= Synthetic Construct
tcccagaaagccagaactatg

SEQ ID NO: 151 - DNA, 23 bp, Artificial Sequence
  Description of Artificial Sequence; Note= Synthetic Construct
catagttctttaagcctccatcg

SEQ ID NO: 152 - DNA, 20 bp, Artificial Sequence
  Description of Artificial Sequence; Note= Synthetic Construct
ccaaccaaatcctctccctt

SEQ ID NO: 153 - DNA, 20 bp, Artificial Sequence
  Description of Artificial Sequence; Note= Synthetic Construct
tgaggagacagcactgcaag

SEQ ID NO: 154 - DNA, 25 bp, Artificial Sequence
  Description of Artificial Sequence; Note= Synthetic Construct
aaagggcacctcataattaactctt

SEQ ID NO: 155 - DNA, 20 bp, Artificial Sequence
  Description of Artificial Sequence; Note= Synthetic Construct
ctctgtggcttgcccattac

SEQ ID NO: 156 - DNA, 22 bp, Artificial Sequence
  Description of Artificial Sequence; Note= Synthetic Construct
tgaagggtacgttgagatgatg

SEQ ID NO: 157 - DNA, 20 bp, Artificial Sequence
  Description of Artificial Sequence; Note= Synthetic Construct
gaaaatagccacctccacca

SEQ ID NO: 158 - DNA, 20 bp, Artificial Sequence
  Description of Artificial Sequence; Note= Synthetic Construct
atatcacgccaaaaggatgc

SEQ ID NO: 159 - DNA, 20 bp, Artificial Sequence
  Description of Artificial Sequence; Note= Synthetic Construct
tcactcatagccaaggtgga

SEQ ID NO: 160 - DNA, 20 bp, Artificial Sequence
  Description of Artificial Sequence; Note= Synthetic Construct
aagcaggtaagcctggatga

SEQ ID NO: 161 - DNA, 20 bp, Artificial Sequence
  Description of Artificial Sequence; Note= Synthetic Construct
ttacaactcctgattcccgc

SEQ ID NO: 162 - DNA, 20 bp, Artificial Sequence
  Description of Artificial Sequence; Note= Synthetic Construct
tcacaaatgtgatggggcta

SEQ ID NO: 163 - DNA, 20 bp, Artificial Sequence
  Description of Artificial Sequence; Note= Synthetic Construct
atatggaacgcattttgggt

SEQ ID NO: 164 - DNA, 20 bp, Artificial Sequence
  Description of Artificial Sequence; Note= Synthetic Construct
cctgtgtggaactactcgca

SEQ ID NO: 165 - DNA, 20 bp, Artificial Sequence
  Description of Artificial Sequence; Note= Synthetic Construct
gccaggaggaaactacacca

SEQ ID NO: 166 - DNA, 20 bp, Artificial Sequence
  Description of Artificial Sequence; Note= Synthetic Construct
ggtccagcgtcacataaagg

SEQ ID NO: 167 - DNA, 20 bp, Artificial Sequence
  Description of Artificial Sequence; Note= Synthetic Construct
actcccaagcagtagcagga

SEQ ID NO: 168 - DNA, 21 bp, Artificial Sequence
  Description of Artificial Sequence; Note= Synthetic Construct
catgccatgtgatgtttatgc

SEQ ID NO: 169 - DNA, 20 bp, Artificial Sequence
  Description of Artificial Sequence; Note= Synthetic Construct
agcccatgaactgtgtttcc

SEQ ID NO: 170 - DNA, 22 bp, Artificial Sequence
  Description of Artificial Sequence; Note= Synthetic Construct
agcaatgaggatgattgattga

SEQ ID NO: 171 - DNA, 20 bp, Artificial Sequence
  Description of Artificial Sequence; Note= Synthetic Construct
gggcacttatactctgggca

SEQ ID NO: 172 - DNA, 20 bp, Artificial Sequence
  Description of Artificial Sequence; Note= Synthetic Construct
cgccttctctctcaagttgg

SEQ ID NO: 173 - DNA, 20 bp, Artificial Sequence
  Description of Artificial Sequence; Note= Synthetic Construct
tcaactaaggctgaatggca

SEQ ID NO: 174 - DNA, 20 bp, Artificial Sequence
  Description of Artificial Sequence; Note= Synthetic Construct
atgcccagaataatccatgc

SEQ ID NO: 175 - DNA, 27 bp, Artificial Sequence
  Description of Artificial Sequence; Note= Synthetic Construct
ccatatctagaagctttattctgtttt

SEQ ID NO: 176 - DNA, 23 bp, Artificial Sequence
  Description of Artificial Sequence; Note= Synthetic Construct
gaatctgctttacagtggttgag

SEQ ID NO: 177 - DNA, 20 bp, Artificial Sequence
  Description of Artificial Sequence; Note= Synthetic Construct
tgatcagatggggattgaca

SEQ ID NO: 178 - DNA, 20 bp, Artificial Sequence
  Description of Artificial Sequence; Note= Synthetic Construct
ttcattaaagccacaaccca

SEQ ID NO: 179 - DNA, 20 bp, Artificial Sequence
  Description of Artificial Sequence; Note= Synthetic Construct
gcatacagggtgccagactt

SEQ ID NO: 180 - DNA, 20 bp, Artificial Sequence
  Description of Artificial Sequence; Note= Synthetic Construct
tagaccagctgggtcgacat

SEQ ID NO: 181 - DNA, 20 bp, Artificial Sequence
  Description of Artificial Sequence; Note= Synthetic Construct
ctcagtcatgctctgtggga

SEQ ID NO: 182 - DNA, 20 bp, Artificial Sequence
  Description of Artificial Sequence; Note= Synthetic Construct
atcaaaacaaccccatggaa

SEQ ID NO: 183 - DNA, 20 bp, Artificial Sequence
  Description of Artificial Sequence; Note= Synthetic Construct
caatagccccattgctcagt

SEQ ID NO: 184 - DNA, 20 bp, Artificial Sequence
  Description of Artificial Sequence; Note= Synthetic Construct
aagagggcacaagctttgaa

SEQ ID NO: 185 - DNA, 20 bp, Artificial Sequence
  Description of Artificial Sequence; Note= Synthetic Construct
cgttctgcaagaatcccaat

SEQ ID NO: 186 - DNA, 20 bp, Artificial Sequence
  Description of Artificial Sequence; Note= Synthetic Construct
tctgaccataaaagcgtgga

SEQ ID NO: 187 - DNA, 22 bp, Artificial Sequence
  Description of Artificial Sequence; Note= Synthetic Construct
cactgtgctattctggtttgga

SEQ ID NO: 188 - DNA, 24 bp, Artificial Sequence
  Description of Artificial Sequence; Note= Synthetic Construct
tttatgcttctttgcaaactactg

SEQ ID NO: 189 - DNA, 20 bp, Artificial Sequence
  Description of Artificial Sequence; Note= Synthetic Construct
attttaatttggatgcccca

SEQ ID NO: 190 - DNA, 20 bp, Artificial Sequence
  Description of Artificial Sequence; Note= Synthetic Construct
tcttcttctgctgggtgaca

SEQ ID NO: 191 - DNA, 27 bp, Artificial Sequence
  Description of Artificial Sequence; Note= Synthetic Construct
ttactcttgctattcaaactaattcaa

SEQ ID NO: 192 - DNA, 20 bp, Artificial Sequence
  Description of Artificial Sequence; Note= Synthetic Construct
ttttctgcaggcggtagagt

SEQ ID NO: 193 - DNA, 20 bp, Artificial Sequence
  Description of Artificial Sequence; Note= Synthetic Construct
gctaaaaacgtaccaggcca

SEQ ID NO: 194 - DNA, 20 bp, Artificial Sequence
  Description of Artificial Sequence; Note= Synthetic Construct
ggagcagcctatcaggtcag

SEQ ID NO: 195 - DNA, 23 bp, Artificial Sequence
  Description of Artificial Sequence; Note= Synthetic Construct
tccagttgacctctttaatctgc

SEQ ID NO: 196 - DNA, 22 bp, Artificial Sequence
  Description of Artificial Sequence; Note= Synthetic Construct
ccgtgatgatccttaacatttc

SEQ ID NO: 197 - DNA, 23 bp, Artificial Sequence
  Description of Artificial Sequence; Note= Synthetic Construct
tggcatagataccaatgaatcag

SEQ ID NO: 198 - DNA, 24 bp, Artificial Sequence
  Description of Artificial Sequence; Note= Synthetic Construct
tgtatcccatagaacactggaaaa

SEQ ID NO: 199 - DNA, 20 bp, Artificial Sequence
  Description of Artificial Sequence; Note= Synthetic Construct
aggactatgggcattggttg

SEQ ID NO: 200 - DNA, 24 bp, Artificial Sequence
  Description of Artificial Sequence; Note= Synthetic Construct
ttttcctaaaagtcttcactgcaa

SEQ ID NO: 201 - DNA, 20 bp, Artificial Sequence
  Description of Artificial Sequence; Note= Synthetic Construct
tgctcatctcattggtctgc

SEQ ID NO: 202 - DNA, 22 bp, Artificial Sequence
  Description of Artificial Sequence; Note= Synthetic Construct
caatgaagcaaaattgaaaagg

SEQ ID NO: 203 - DNA, 20 bp, Artificial Sequence
  Description of Artificial Sequence; Note= Synthetic Construct
aagtgccttcattctgggag

SEQ ID NO: 204 - DNA, 21 bp, Artificial Sequence
  Description of Artificial Sequence; Note= Synthetic Construct
gaaaccattacgggaattcat

SEQ ID NO: 205 - DNA, 21 bp, Artificial Sequence
  Description of Artificial Sequence; Note= Synthetic Construct
ggattttgaccgctatttgaa

SEQ ID NO: 206 - DNA, 20 bp, Artificial Sequence
  Description of Artificial Sequence; Note= Synthetic Construct
gttggccgatcaggtagaaa

SEQ ID NO: 207 - DNA, 20 bp, Artificial Sequence
  Description of Artificial Sequence; Note= Synthetic Construct
gtggttttgggattctgcaa

SEQ ID NO: 208 - DNA, 25 bp, Artificial Sequence
  Description of Artificial Sequence; Note= Synthetic Construct
cagtgcatctatctaacatctgctc

SEQ ID NO: 209 - DNA, 20 bp, Artificial Sequence
  Description of Artificial Sequence; Note= Synthetic Construct
aatagttccggggtgactga

SEQ ID NO: 210 - DNA, 20 bp, Artificial Sequence
  Description of Artificial Sequence; Note= Synthetic Construct
ggaggggacttattcaagcc

SEQ ID NO: 211 - DNA, 20 bp, Artificial Sequence
  Description of Artificial Sequence; Note= Synthetic Construct
tggcttggaatggttttagg

SEQ ID NO: 212 - DNA, 20 bp, Artificial Sequence
  Description of Artificial Sequence; Note= Synthetic Construct
gattttacccatccgcagtt

SEQ ID NO: 213 - DNA, 21 bp, Artificial Sequence
  Description of Artificial Sequence; Note= Synthetic Construct
ttgcttgtctctttgcttttc

SEQ ID NO: 214 - DNA, 21 bp, Artificial Sequence
  Description of Artificial Sequence; Note= Synthetic Construct
catacggccagtttttgaaga

SEQ ID NO: 215 - DNA, 20 bp, Artificial Sequence
  Description of Artificial Sequence; Note= Synthetic Construct
tcgatgggcaaacatctgta

SEQ ID NO: 216 - DNA, 24 bp, Artificial Sequence
  Description of Artificial Sequence; Note= Synthetic Construct
ttgaaaaacaaagttgaaaatcca

SEQ ID NO: 217 - DNA, 20 bp, Artificial Sequence
  Description of Artificial Sequence; Note= Synthetic Construct
gaacttttgatcctttgcgg

SEQ ID NO: 218 - DNA, 20 bp, Artificial Sequence
  Description of Artificial Sequence; Note= Synthetic Construct
tcaccaccattctccaacaa

SEQ ID NO: 219 - DNA, 22 bp, Artificial Sequence
  Description of Artificial Sequence; Note= Synthetic Construct
tgttgagattactttcccttgc

SEQ ID NO: 220 - DNA, 22 bp, Artificial Sequence
  Description of Artificial Sequence; Note= Synthetic Construct
ttgcgatagtgatttcttgtga

SEQ ID NO: 221 - DNA, 21 bp, Artificial Sequence
  Description of Artificial Sequence; Note= Synthetic Construct
aacagggaaaatagtgctgct

SEQ ID NO: 222 - DNA, 21 bp, Artificial Sequence
  Description of Artificial Sequence; Note= Synthetic Construct
ggcatccctagtcagtcacag

SEQ ID NO: 223 - DNA, 20 bp, Artificial Sequence
  Description of Artificial Sequence; Note= Synthetic Construct
tcatgaaaatggctcatgct

SEQ ID NO: 224 - DNA, 20 bp, Artificial Sequence
  Description of Artificial Sequence; Note= Synthetic Construct
ccacatcccattttcttcca

SEQ ID NO: 225 - DNA, 20 bp, Artificial Sequence
  Description of Artificial Sequence; Note= Synthetic Construct
ttgttgtgacgcaagtctga

SEQ ID NO: 226 - DNA, 20 bp, Artificial Sequence
  Description of Artificial Sequence; Note= Synthetic Construct
ttgcgcttagctaaatcctt

SEQ ID NO: 227 - DNA, 20 bp, Artificial Sequence
  Description of Artificial Sequence; Note= Synthetic Construct
ggctggtgatagaggcttgt

SEQ ID NO: 228 - DNA, 26 bp, Artificial Sequence
  Description of Artificial Sequence; Note= Synthetic Construct
tcacaaaattattatgaggacaaaaa

SEQ ID NO: 229 - DNA, 20 bp, Artificial Sequence
  Description of Artificial Sequence; Note= Synthetic Construct
atgtgtaaggtccctggcat

SEQ ID NO: 230 - DNA, 19 bp, Artificial Sequence
  Description of Artificial Sequence; Note= Synthetic Construct
ttttcatttgctcaatggg

SEQ ID NO: 231 - DNA, 27 bp, Artificial Sequence
  Description of Artificial Sequence; Note= Synthetic Construct
tcagaaaaatacatatggagtgttaaa

SEQ ID NO: 232 - DNA, 20 bp, Artificial Sequence
  Description of Artificial Sequence; Note= Synthetic Construct
aaggaataagcaaatcgcca

SEQ ID NO: 233 - DNA, 26 bp, Artificial Sequence
  Description of Artificial Sequence; Note= Synthetic Construct
gcctcaagaactacttagagacatcc

SEQ ID NO: 234 - DNA, 21 bp, Artificial Sequence
  Description of Artificial Sequence; Note= Synthetic Construct
aggcaatgttttgtcagttcc

SEQ ID NO: 235 - DNA, 20 bp, Artificial Sequence
  Description of Artificial Sequence; Note= Synthetic Construct
cccactggattcatgccata

SEQ ID NO: 236 - DNA, 27 bp, Artificial Sequence
  Description of Artificial Sequence; Note= Synthetic Construct
ttttaggatcaaaataagatgaatgtg

SEQ ID NO: 237 - DNA, 22 bp, Artificial Sequence
  Description of Artificial Sequence; Note= Synthetic Construct
tgagtgtatctgatccccatga

SEQ ID NO: 238 - DNA, 23 bp, Artificial Sequence
  Description of Artificial Sequence; Note= Synthetic Construct
tctgatccccatgagttattttc

SEQ ID NO: 239 - DNA, 23 bp, Artificial Sequence
  Description of Artificial Sequence; Note= Synthetic Construct
tttatggaagagactggagttca

SEQ ID NO: 240 - DNA, 27 bp, Artificial Sequence
  Description of Artificial Sequence; Note= Synthetic Construct
ggagaaaatttataggattttatgacc

SEQ ID NO: 241 - DNA, 27 bp, Artificial Sequence
  Description of Artificial Sequence; Note= Synthetic Construct
tttcttaatgacttttgattgtagagg

SEQ ID NO: 242 - DNA, 21 bp, Artificial Sequence
  Description of Artificial Sequence; Note= Synthetic Construct
gaagccatttaaaccctttgc

SEQ ID NO: 243 - DNA, 20 bp, Artificial Sequence
  Description of Artificial Sequence; Note= Synthetic Construct
gcaaaaatgctccttggtgt

SEQ ID NO: 244 - DNA, 20 bp, Artificial Sequence
  Description of Artificial Sequence; Note= Synthetic Construct
cagtgtctggcattggattg

SEQ ID NO: 245 - DNA, 20 bp, Artificial Sequence
  Description of Artificial Sequence; Note= Synthetic Construct
ggaggaacattcgacctgag

SEQ ID NO: 246 - DNA, 25 bp, Artificial Sequence
  Description of Artificial Sequence; Note= Synthetic Construct
tcctacctacctccaaatagtcaaa

SEQ ID NO: 247 - DNA, 22 bp, Artificial Sequence
  Description of Artificial Sequence; Note= Synthetic Construct
cccatagggaagaaataaatcg

SEQ ID NO: 248 - DNA, 24 bp, Artificial Sequence
  Description of Artificial Sequence; Note= Synthetic Construct
catacatttgggagaatgattcag

SEQ ID NO: 249 - DNA, 20 bp, Artificial Sequence
  Description of Artificial Sequence; Note= Synthetic Construct
tcctgtgttggatgaatgga

SEQ ID NO: 250 - DNA, 20 bp, Artificial Sequence
  Description of Artificial Sequence; Note= Synthetic Construct
gccacaatacatgtgccaat

SEQ ID NO: 251 - DNA, 20 bp, Artificial Sequence
  Description of Artificial Sequence; Note= Synthetic Construct
accgctgcaaaatgctactc

SEQ ID NO: 252 - DNA, 22 bp, Artificial Sequence
  Description of Artificial Sequence; Note= Synthetic Construct
ctgaataagcagagcctcactg

SEQ ID NO: 253 - DNA, 20 bp, Artificial Sequence
  Description of Artificial Sequence; Note= Synthetic Construct
acgatgtcatctgccctagc

SEQ ID NO: 254 - DNA, 20 bp, Artificial Sequence
  Description of Artificial Sequence; Note= Synthetic Construct
tcatggtcctgaaaagcaca

SEQ ID NO: 255 - DNA, 23 bp, Artificial Sequence
  Description of Artificial Sequence; Note= Synthetic Construct
tcatagttacccaacaatgaagc

SEQ ID NO: 256 - DNA, 27 bp, Artificial Sequence
  Description of Artificial Sequence; Note= Synthetic Construct
agtttcattgagattagttttaagtgg

SEQ ID NO: 257 - DNA, 27 bp, Artificial Sequence
  Description of Artificial Sequence; Note= Synthetic Construct
cgcaatattctatatgaaaataccact

SEQ ID NO: 258 - DNA, 23 bp, Artificial Sequence
  Description of Artificial Sequence; Note= Synthetic Construct
tgagtgatggatttgaacagaaa

SEQ ID NO: 259 - DNA, 22 bp, Artificial Sequence
  Description of Artificial Sequence; Note= Synthetic Construct
ccctttgtattttctgcatgtg

SEQ ID NO: 260 - DNA, 20 bp, Artificial Sequence
  Description of Artificial Sequence; Note= Synthetic Construct
gggaggagtggcgtttatct

SEQ ID NO: 261 - DNA, 21 bp, Artificial Sequence
  Description of Artificial Sequence; Note= Synthetic Construct
tgcatgtatgttcagctctgg

SEQ ID NO: 262 - DNA, 20 bp, Artificial Sequence
  Description of Artificial Sequence; Note= Synthetic Construct
tcaaaagaaaattgctgggc

SEQ ID NO: 263 - DNA, 20 bp, Artificial Sequence
  Description of Artificial Sequence; Note= Synthetic Construct
caggtgcccctaaaaatgtg

SEQ ID NO: 264 - DNA, 21 bp, Artificial Sequence
  Description of Artificial Sequence; Note= Synthetic Construct
gcaacacatcgttcaaaatca

SEQ ID NO: 265 - DNA, 20 bp, Artificial Sequence
  Description of Artificial Sequence; Note= Synthetic Construct
cttcctatacatgggtcccg

SEQ ID NO: 266 - DNA, 22 bp, Artificial Sequence
  Description of Artificial Sequence; Note= Synthetic Construct
caaggaaatgcatcaaatcaaa

SEQ ID NO: 267 - DNA, 20 bp, Artificial Sequence
  Description of Artificial Sequence; Note= Synthetic Construct
gggttattgagcgaggatga

SEQ ID NO: 268 - DNA, 20 bp, Artificial Sequence
  Description of Artificial Sequence; Note= Synthetic Construct
aagcccaaagtgagggaaac

SEQ ID NO: 269 - DNA, 27 bp, Artificial Sequence
  Description of Artificial Sequence; Note= Synthetic Construct
gcttttaacactttctggaaaagtaag

SEQ ID NO: 270 - DNA, 21 bp, Artificial Sequence
  Description of Artificial Sequence; Note= Synthetic Construct
agatttctgaagccaaccaca

SEQ ID NO: 271 - DNA, 20 bp, Artificial Sequence
  Description of Artificial Sequence; Note= Synthetic Construct
caccatttgctacctttggg

SEQ ID NO: 272 - DNA, 22 bp, Artificial Sequence
  Description of Artificial Sequence; Note= Synthetic Construct
ttcagctcatttgtctgaattg

SEQ ID NO: 273 - DNA, 22 bp, Artificial Sequence
  Description of Artificial Sequence; Note= Synthetic Construct
ttgtgtgtacatgctaggtgtg

SEQ ID NO: 274 - DNA, 20 bp, Artificial Sequence
  Description of Artificial Sequence; Note= Synthetic Construct
ccaggcaaactctctcatcc

SEQ ID NO: 275 - DNA, 20 bp, Artificial Sequence
  Description of Artificial Sequence; Note= Synthetic Construct
gggaaattttcacatggagc

SEQ ID NO: 276 - DNA, 21 bp, Artificial Sequence
  Description of Artificial Sequence; Note= Synthetic Construct
cctttaagcaatcatgggtga

SEQ ID NO: 277 - DNA, 25 bp, Artificial Sequence
  Description of Artificial Sequence; Note= Synthetic Construct
tgaatcagaatttttcttgttcgat

SEQ ID NO: 278 - DNA, 20 bp, Artificial Sequence
  Description of Artificial Sequence; Note= Synthetic Construct
taagcgctagggttacaggc

SEQ ID NO: 279 - DNA, 20 bp, Artificial Sequence
  Description of Artificial Sequence; Note= Synthetic Construct
gagggggtgagtgtttcagt

SEQ ID NO: 280 - DNA, 20 bp, Artificial Sequence
  Description of Artificial Sequence; Note= Synthetic Construct
aaagccattcaccatcatca

SEQ ID NO: 281 - DNA, 20 bp, Artificial Sequence
  Description of Artificial Sequence; Note= Synthetic Construct
tcagttgcagttggctatgc

SEQ ID NO: 282 - DNA, 17 bp, Artificial Sequence
  Description of Artificial Sequence; Note= Synthetic Construct
gtgaggttggtttagcc

SEQ ID NO: 283 - DNA, 22 bp, Artificial Sequence
  Description of Artificial Sequence; Note= Synthetic Construct
tctgtttcttttctctgcacca

SEQ ID NO: 284 - DNA, 23 bp, Artificial Sequence
  Description of Artificial Sequence; Note= Synthetic Construct
gagtcctttaaagcaatgactcg

SEQ ID NO: 285 - DNA, 20 bp, Artificial Sequence
  Description of Artificial Sequence; Note= Synthetic Construct
tatttgatgggtggttggct

SEQ ID NO: 286 - DNA, 20 bp, Artificial Sequence
  Description of Artificial Sequence; Note= Synthetic Construct
ccgttgtcatgcaacacttt

SEQ ID NO: 287 - DNA, 22 bp, Artificial Sequence
  Description of Artificial Sequence; Note= Synthetic Construct
tcatgaataagagtttggctca

SEQ ID NO: 288 - DNA, 26 bp, Artificial Sequence
  Description of Artificial Sequence; Note= Synthetic Construct
ttaggctgaatagtgagagtaatgtg

SEQ ID NO: 289 - DNA, 20 bp, Artificial Sequence
  Description of Artificial Sequence; Note= Synthetic Construct
cggaatgtctccatttgagc

SEQ ID NO: 290 - DNA, 22 bp, Artificial Sequence
  Description of Artificial Sequence; Note= Synthetic Construct
tgctttgcaactatataagccc

SEQ ID NO: 291 - DNA, 21 bp, Artificial Sequence
  Description of Artificial Sequence; Note= Synthetic Construct
tgttgttcatcatcctagcca

SEQ ID NO: 292 - DNA, 20 bp, Artificial Sequence
  Description of Artificial Sequence; Note= Synthetic Construct
agcctgggtgacagtgagac

SEQ ID NO: 293 - DNA, 20 bp, Artificial Sequence
  Description of Artificial Sequence; Note= Synthetic Construct
tttgtcctgaaaggtgggtt

SEQ ID NO: 294 - DNA, 20 bp, Artificial Sequence
  Description of Artificial Sequence; Note= Synthetic Construct
agaagtctgagccaagtccg

SEQ ID NO: 295 - DNA, 20 bp, Artificial Sequence
  Description of Artificial Sequence; Note= Synthetic Construct
tgtcattcttgcatgccttc

SEQ ID NO: 296 - DNA, 20 bp, Artificial Sequence
  Description of Artificial Sequence; Note= Synthetic Construct
cctccttgtccaaataccga

SEQ ID NO: 297 - DNA, 20 bp, Artificial Sequence
  Description of Artificial Sequence; Note= Synthetic Construct
caatacgccaagaaaaggga

SEQ ID NO: 298 - DNA, 24 bp, Artificial Sequence
  Description of Artificial Sequence; Note= Synthetic Construct
tgatgtcttaatatgcatgtctcc

SEQ ID NO: 299 - DNA, 20 bp, Artificial Sequence
  Description of Artificial Sequence; Note= Synthetic Construct
cctctgttttgtggctctca

SEQ ID NO: 300 - DNA, 20 bp, Artificial Sequence
  Description of Artificial Sequence; Note= Synthetic Construct
gccaaaagagatggacgatt

SEQ ID NO: 301 - DNA, 20 bp, Artificial Sequence
  Description of Artificial Sequence; Note= Synthetic Construct
aacacagcgctttcctcatt

SEQ ID NO: 302 - DNA, 22 bp, Artificial Sequence
  Description of Artificial Sequence; Note= Synthetic Construct
ttcctcctcacagataactccc

SEQ ID NO: 303 - DNA, 22 bp, Artificial Sequence
  Description of Artificial Sequence; Note= Synthetic Construct
gggctgtatcaaaatttatgcc

SEQ ID NO: 304 - DNA, 22 bp, Artificial Sequence
  Description of Artificial Sequence; Note= Synthetic Construct
ttgtgggaagataacactgcac

SEQ ID NO: 305 - DNA, 20 bp, Artificial Sequence
  Description of Artificial Sequence; Note= Synthetic Construct
actggcactgcaccctaaag

SEQ ID NO: 306 - DNA, 24 bp, Artificial Sequence
  Description of Artificial Sequence; Note= Synthetic Construct
aatttgaaaatgtttagatgggaa

SEQ ID NO: 307 - DNA, 20 bp, Artificial Sequence
  Description of Artificial Sequence; Note= Synthetic Construct
atcctttgtgtttggccttg

SEQ ID NO: 308 - DNA, 20 bp, Artificial Sequence
  Description of Artificial Sequence; Note= Synthetic Construct
atccaattggccttcctctt

SEQ ID NO: 309 - DNA, 20 bp, Artificial Sequence
  Description of Artificial Sequence; Note= Synthetic Construct
cgcatttatctttgtgcctg

SEQ ID NO: 310 - DNA, 20 bp, Artificial Sequence
  Description of Artificial Sequence; Note= Synthetic Construct
cgcaaagattgactcccact

SEQ ID NO: 311 - DNA, 20 bp, Artificial Sequence
  Description of Artificial Sequence; Note= Synthetic Construct
gggcctttctgcttgtaaga

SEQ ID NO: 312 - DNA, 22 bp, Artificial Sequence
  Description of Artificial Sequence; Note= Synthetic Construct
caaagacctataggccctctca

SEQ ID NO: 313 - DNA, 20 bp, Artificial Sequence
  Description of Artificial Sequence; Note= Synthetic Construct
gttgtcaaagggcaaaagga

SEQ ID NO: 314 - DNA, 20 bp, Artificial Sequence
  Description of Artificial Sequence; Note= Synthetic Construct
agctgaggaatggtgacagg

SEQ ID NO: 315 - DNA, 20 bp, Artificial Sequence
  Description of Artificial Sequence; Note= Synthetic Construct
tgtggttcacgtttggtgtt

SEQ ID NO: 316 - DNA, 23 bp, Artificial Sequence
  Description of Artificial Sequence; Note= Synthetic Construct
gagagcaatctacattctggctc

SEQ ID NO: 317 - DNA, 20 bp, Artificial Sequence
  Description of Artificial Sequence; Note= Synthetic Construct
tggttgaatttccattgcat

SEQ ID NO: 318 - DNA, 20 bp, Artificial Sequence
  Description of Artificial Sequence; Note= Synthetic Construct
ttgacaaggaatggcacaaa

SEQ ID NO: 319 - DNA, 22 bp, Artificial Sequence
  Description of Artificial Sequence; Note= Synthetic Construct
gcacaaattagaagtaacccca

SEQ ID NO: 320 - DNA, 20 bp, Artificial Sequence
  Description of Artificial Sequence; Note= Synthetic Construct
cctgctgcagatggagattt

SEQ ID NO: 321 - DNA, 20 bp, Artificial Sequence
  Description of Artificial Sequence; Note= Synthetic Construct
agctgtgaaaagccagccta

SEQ ID NO: 322 - DNA, 22 bp, Artificial Sequence
  Description of Artificial Sequence; Note= Synthetic Construct
gggtagctctttggatatcagg

SEQ ID NO: 323 - DNA, 21 bp, Artificial Sequence
  Description of Artificial Sequence; Note= Synthetic Construct
agctgagtttttcttccctcc

SEQ ID NO: 324 - DNA, 21 bp, Artificial Sequence
  Description of Artificial Sequence; Note= Synthetic Construct
gaagcctacagttgagagcca

SEQ ID NO: 325 - DNA, 26 bp, Artificial Sequence
  Description of Artificial Sequence; Note= Synthetic Construct
ttgagtagcctagtaagcttgtatgt

SEQ ID NO: 326 - DNA, 21 bp, Artificial Sequence
  Description of Artificial Sequence; Note= Synthetic Construct
aaagtggcaactggacatcag

SEQ ID NO: 327 - DNA, 20 bp, Artificial Sequence
  Description of Artificial Sequence; Note= Synthetic Construct
gatcaaaggggacgtcttca

SEQ ID NO: 328 - DNA, 20 bp, Artificial Sequence
  Description of Artificial Sequence; Note= Synthetic Construct
atgtccagttgccactttcc

SEQ ID NO: 329 - DNA, 20 bp, Artificial Sequence
  Description of Artificial Sequence; Note= Synthetic Construct
cgatgggaattttccagaga

SEQ ID NO: 330 - DNA, 20 bp, Artificial Sequence
  Description of Artificial Sequence; Note= Synthetic Construct
ccggaaatgtttaaaagcca

SEQ ID NO: 331 - DNA, 20 bp, Artificial Sequence
  Description of Artificial Sequence; Note= Synthetic Construct
tggtctaccacacactgcct

SEQ ID NO: 332 - DNA, 41 bp, Artificial Sequence
  Description of Artificial Sequence; Note= Synthetic Construct
aagatcacgtttccactccctggtagatcacaacctcagca

SEQ ID NO: 333 - DNA, 21 bp, Artificial Sequence
  Description of Artificial Sequence; Note= Synthetic Construct
ctgcaaatggagctaaacaga

SEQ ID NO: 334 - DNA, 20 bp, Artificial Sequence
  Description of Artificial Sequence; Note= Synthetic Construct
gcctcttttgcttgctgttc

SEQ ID NO: 335 - DNA, 20 bp, Artificial Sequence
  Description of Artificial Sequence; Note= Synthetic Construct
tcactttgcaggcacatacc

SEQ ID NO: 336 - DNA, 22 bp, Artificial Sequence
  Description of Artificial Sequence; Note= Synthetic Construct
gggagcacaattcagatacaaa

SEQ ID NO: 337 - DNA, 20 bp, Artificial Sequence
  Description of Artificial Sequence; Note= Synthetic Construct
acaagttttctgtgggccag

SEQ ID NO: 338 - DNA, 23 bp, Artificial Sequence
  Description of Artificial Sequence; Note= Synthetic Construct
tgtatggatttcttcttcccttt

SEQ ID NO: 339 - DNA, 19 bp, Artificial Sequence
  Description of Artificial Sequence; Note= Synthetic Construct
gaaacatgttgccctcacg

SEQ ID NO: 340 - DNA, 20 bp, Artificial Sequence
  Description of Artificial Sequence; Note= Synthetic Construct
gctgcaagtggagaggtgac

SEQ ID NO: 341 - DNA, 20 bp, Artificial Sequence
  Description of Artificial Sequence; Note= Synthetic Construct
gggactacaaaggattgcca

SEQ ID NO: 342 - DNA, 21 bp, Artificial Sequence
  Description of Artificial Sequence; Note= Synthetic Construct
ttcttcctggaaactggtgaa

SEQ ID NO: 343 - DNA, 27 bp, Artificial Sequence
  Description of Artificial Sequence; Note= Synthetic Construct
gcacactttagtttacaatctttcttt

SEQ ID NO: 344 - DNA, 21 bp, Artificial Sequence
  Description of Artificial Sequence; Note= Synthetic Construct
aacaatggcaggttttacacg

SEQ ID NO: 345 - DNA, 20 bp, Artificial Sequence
  Description of Artificial Sequence; Note= Synthetic Construct
aagcaggtaagcctggatga

SEQ ID NO: 346 - DNA, 20 bp, Artificial Sequence
  Description of Artificial Sequence; Note= Synthetic Construct
ggcaggcttgagttttcatt

SEQ ID NO: 347 - DNA, 20 bp, Artificial Sequence
  Description of Artificial Sequence; Note= Synthetic Construct
tactccttcacagggatggg

SEQ ID NO: 348 - DNA, 20 bp, Artificial Sequence
  Description of Artificial Sequence; Note= Synthetic Construct
acattcagcttcctgctgct

SEQ ID NO: 349 - DNA, 22 bp, Artificial Sequence
  Description of Artificial Sequence; Note= Synthetic Construct
aacctgtctaatccaccaagaa

SEQ ID NO: 350 - DNA, 20 bp, Artificial Sequence
  Description of Artificial Sequence; Note= Synthetic Construct
caggtatcaacccagaagcc

SEQ ID NO: 351 - DNA, 22 bp, Artificial Sequence
  Description of Artificial Sequence; Note= Synthetic Construct
gagctttgggttttcttttgaa

SEQ ID NO: 352 - DNA, 20 bp, Artificial Sequence
  Description of Artificial Sequence; Note= Synthetic Construct
tttggagagtgggctgacat

SEQ ID NO: 353 - DNA, 24 bp, Artificial Sequence
  Description of Artificial Sequence; Note= Synthetic Construct
ggtggttataaagaacacaacacg

SEQ ID NO: 354 - DNA, 47 bp, Artificial Sequence
  Description of Artificial Sequence; Note= Synthetic Construct
aaatcagaggtaaatagagtgcataaaggggaaggggtagttaggag

SEQ ID NO: 355 - DNA, 20 bp, Artificial Sequence
  Description of Artificial Sequence; Note= Synthetic Construct
tactcattgcagtcgcaagc

SEQ ID NO: 356 - DNA, 21 bp, Artificial Sequence
  Description of Artificial Sequence; Note= Synthetic Construct
tgatgatgccaacagtctgaa

SEQ ID NO: 357 - DNA, 26 bp, Artificial Sequence
  Description of Artificial Sequence; Note= Synthetic Construct
gcataattcacaactgaaatttagga

SEQ ID NO: 358 - DNA, 19 bp, Artificial Sequence
  Description of Artificial Sequence; Note= Synthetic Construct
gtagaggcccccggatatt

SEQ ID NO: 359 - DNA, 27 bp, Artificial Sequence
  Description of Artificial Sequence; Note= Synthetic Construct
aaaacagaataaagcttctagatatgg

SEQ ID NO: 360 - DNA, 23 bp, Artificial Sequence
  Description of Artificial Sequence; Note= Synthetic Construct
gaatctgctttacagtggttgag

SEQ ID NO: 361 - DNA, 24 bp, Artificial Sequence
  Description of Artificial Sequence; Note= Synthetic Construct
ggtgtcttcataataatcagctcc

SEQ ID NO: 362 - DNA, 19 bp, Artificial Sequence
  Description of Artificial Sequence; Note= Synthetic Construct
ctcacaacaaaagccccaa

SEQ ID NO: 363 - DNA, 20 bp, Artificial Sequence
  Description of Artificial Sequence; Note= Synthetic Construct
tcagccaaaatttcagtgtg

SEQ ID NO: 364 - DNA, 20 bp, Artificial Sequence
  Description of Artificial Sequence; Note= Synthetic Construct
gcagagtttgaagagctcgg

SEQ ID NO: 365 - DNA, 20 bp, Artificial Sequence
  Description of Artificial Sequence; Note= Synthetic Construct
ccaataagttgcctgcccta

SEQ ID NO: 366 - DNA, 26 bp, Artificial Sequence
  Description of Artificial Sequence; Note= Synthetic Construct
tgtgaaggagaaaaataaatagcaaa

SEQ ID NO: 367 - DNA, 20 bp, Artificial Sequence
  Description of Artificial Sequence; Note= Synthetic Construct
tcagcaaaccttgcattttt

SEQ ID NO: 368 - DNA, 20 bp, Artificial Sequence
  Description of Artificial Sequence; Note= Synthetic Construct
cacgctcctgcatcagaata

SEQ ID NO: 369 - DNA, 19 bp, Artificial Sequence
  Description of Artificial Sequence; Note= Synthetic Construct
caaagcctccattcattgt

SEQ ID NO: 370 - DNA, 26 bp, Artificial Sequence
  Description of Artificial Sequence; Note= Synthetic Construct
tgatttcccatttaatacacattttt

SEQ ID NO: 371 - DNA, 21 bp, Artificial Sequence
  Description of Artificial Sequence; Note= Synthetic Construct
gcagttctcagcttctttcca

SEQ ID NO: 372 - DNA, 20 bp, Artificial Sequence
  Description of Artificial Sequence; Note= Synthetic Construct
gctctgtcatgtgcccacta

SEQ ID NO: 373 - DNA, 21 bp, Artificial Sequence
  Description of Artificial Sequence; Note= Synthetic Construct
ctgccctaactctcaagttgc

SEQ ID NO: 374 - DNA, 20 bp, Artificial Sequence
  Description of Artificial Sequence; Note= Synthetic Construct
attggtttgaaggtcccaga

SEQ ID NO: 375 - DNA, 20 bp, Artificial Sequence
  Description of Artificial Sequence; Note= Synthetic Construct
ttccaaggaaagactggctg

SEQ ID NO: 376 - DNA, 20 bp, Artificial Sequence
  Description of Artificial Sequence; Note= Synthetic Construct
accagctctatgccaaggtg

SEQ ID NO: 377 - DNA, 20 bp, Artificial Sequence
  Description of Artificial Sequence; Note= Synthetic Construct
tcaatgagggagaaagtgcc

SEQ ID NO: 378 - DNA, 20 bp, Artificial Sequence
  Description of Artificial Sequence; Note= Synthetic Construct
gttgaggaagggctgcatta

SEQ ID NO: 379 - DNA, 20 bp, Artificial Sequence
  Description of Artificial Sequence; Note= Synthetic Construct
gcattgcaagtcttggatca

SEQ ID NO: 380 - DNA, 21 bp, Artificial Sequence
  Description of Artificial Sequence; Note= Synthetic Construct
tcaatatactgagcagccctc

SEQ ID NO: 381 - DNA, 20 bp, Artificial Sequence
  Description of Artificial Sequence; Note= Synthetic Construct
agctccaagtgtcaggaagc

SEQ ID NO: 382 - DNA, 22 bp, Artificial Sequence
  Description of Artificial Sequence; Note= Synthetic Construct
tcagtattctccagtgagcagg

SEQ ID NO: 383 - DNA, 20 bp, Artificial Sequence
  Description of Artificial Sequence; Note= Synthetic Construct
ctccttaggcacggtcatgt

SEQ ID NO: 384 - DNA, 21 bp, Artificial Sequence
  Description of Artificial Sequence; Note= Synthetic Construct
cacgagagaacaggaagctca

SEQ ID NO: 385 - DNA, 21 bp, Artificial Sequence
  Description of Artificial Sequence; Note= Synthetic Construct
gcttcctgttctctcgtgttc

SEQ ID NO: 386 - DNA, 18 bp, Artificial Sequence
  Description of Artificial Sequence; Note= Synthetic Construct
cttccactcctggccctt

SEQ ID NO: 387 - DNA, 20 bp, Artificial Sequence
  Description of Artificial Sequence; Note= Synthetic Construct
cctggtctcaggaatctcca

SEQ ID NO: 388 - DNA, 20 bp, Artificial Sequence
  Description of Artificial Sequence; Note= Synthetic Construct
gagagagggtgaggttgacg

SEQ ID NO: 389 - DNA, 20 bp, Artificial Sequence
  Description of Artificial Sequence; Note= Synthetic Construct
tcagaagtgacagcgtttgc

SEQ ID NO: 390 - DNA, 20 bp, Artificial Sequence
  Description of Artificial Sequence; Note= Synthetic Construct
tccttccctacatcacccaa

SEQ ID NO: 391 - DNA, 23 bp, Artificial Sequence
  Description of Artificial Sequence; Note= Synthetic Construct
tggcacttggtgatatgataaga

SEQ ID NO: 392 - DNA, 18 bp, Artificial Sequence
  Description of Artificial Sequence; Note= Synthetic Construct
gtgcgagggagaaagtgc

SEQ ID NO: 393 - DNA, 20 bp, Artificial Sequence
  Description of Artificial Sequence; Note= Synthetic Construct
agagaaatgcctgaatcgtg

SEQ ID NO: 394 - DNA, 20 bp, Artificial Sequence
  Description of Artificial Sequence; Note= Synthetic Construct
agaagacccggaggatgaat

SEQ ID NO: 395 - DNA, 20 bp, Artificial Sequence
  Description of Artificial Sequence; Note= Synthetic Construct
ttgtgggcaggactgtgata

SEQ ID NO: 396 - DNA, 20 bp, Artificial Sequence
  Description of Artificial Sequence; Note= Synthetic Construct
gtgtcaccagaagcaagcag

SEQ ID NO: 397 - DNA, 20 bp, Artificial Sequence
  Description of Artificial Sequence; Note= Synthetic Construct
ctgagccactggccacatta

SEQ ID NO: 398 - DNA, 20 bp, Artificial Sequence
  Description of Artificial Sequence; Note= Synthetic Construct
gactttgggcttctcactgc

SEQ ID NO: 399 - DNA, 21 bp, Artificial Sequence
  Description of Artificial Sequence; Note= Synthetic Construct
aggtcagtttgagagagccat

SEQ ID NO: 400 - DNA, 20 bp, Artificial Sequence
  Description of Artificial Sequence; Note= Synthetic Construct
tgtgggtctggacaacacag

SEQ ID NO: 401 - DNA, 20 bp, Artificial Sequence
  Description of Artificial Sequence; Note= Synthetic Construct
tatccttgtcacttgcacga

SEQ ID NO: 402 - DNA, 20 bp, Artificial Sequence
  Description of Artificial Sequence; Note= Synthetic Construct
aagctggttctgtctcagcc

SEQ ID NO: 403 - DNA, 20 bp, Artificial Sequence
  Description of Artificial Sequence; Note= Synthetic Construct
ggccttgagcatttcacaat

SEQ ID NO: 404 - DNA, 20 bp, Artificial Sequence
  Description of Artificial Sequence; Note= Synthetic Construct
ctccttaagtttccctgggc

SEQ ID NO: 405 - DNA, 20 bp, Artificial Sequence
  Description of Artificial Sequence; Note= Synthetic Construct
ggctggagaggtgtgaagag

SEQ ID NO: 406 - DNA, 20 bp, Artificial Sequence
  Description of Artificial Sequence; Note= Synthetic Construct
gctttccagagccatctgtc

SEQ ID NO: 407 - DNA, 20 bp, Artificial Sequence
  Description of Artificial Sequence; Note= Synthetic Construct
ggcagctctgatcaggaaag

SEQ ID NO: 408 - DNA, 20 bp, Artificial Sequence
  Description of Artificial Sequence; Note= Synthetic Construct
ttgactgcatttcgcatctc

SEQ ID NO: 409 - DNA, 20 bp, Artificial Sequence
  Description of Artificial Sequence; Note= Synthetic Construct
tgaaccatgaccctcctctc

SEQ ID NO: 410 - DNA, 20 bp, Artificial Sequence
  Description of Artificial Sequence; Note= Synthetic Construct
gatgtgcaggcagagaatca

SEQ ID NO: 411 - DNA, 20 bp, Artificial Sequence
  Description of Artificial Sequence; Note= Synthetic Construct
gacctgaagacacacgggtt

SEQ ID NO: 412 - DNA, 20 bp, Artificial Sequence
  Description of Artificial Sequence; Note= Synthetic Construct
cgcactccgcctctactact

SEQ ID NO: 413 - DNA, 20 bp, Artificial Sequence
  Description of Artificial Sequence; Note= Synthetic Construct
cctgggttacagagtaggcg

SEQ ID NO: 414 - DNA, 20 bp, Artificial Sequence
  Description of Artificial Sequence; Note= Synthetic Construct
gcagccactgaaagaagtcc

SEQ ID NO: 415 - DNA, 20 bp, Artificial Sequence
  Description of Artificial Sequence; Note= Synthetic Construct
gagatgcgaaatgcagtcaa

SEQ ID NO: 416 - DNA, 20 bp, Artificial Sequence
  Description of Artificial Sequence; Note= Synthetic Construct
tctgcagacagcctagagca

SEQ ID NO: 417 - DNA, 20 bp, Artificial Sequence
  Description of Artificial Sequence; Note= Synthetic Construct
atggcaaagggagggttact

SEQ ID NO: 418 - DNA, 20 bp, Artificial Sequence
  Description of Artificial Sequence; Note= Synthetic Construct
cccgttgtacatgacccatt

SEQ ID NO: 419 - DNA, 20 bp, Artificial Sequence
  Description of Artificial Sequence; Note= Synthetic Construct
cagcgaacactggattctga

SEQ ID NO: 420 - DNA, 22 bp, Artificial Sequence
  Description of Artificial Sequence; Note= Synthetic Construct
tggctctctcaaactgacctaa

SEQ ID NO: 421 - DNA, 20 bp, Artificial Sequence
  Description of Artificial Sequence; Note= Synthetic Construct
ttgtgggcaggactgtgata

SEQ ID NO: 422 - DNA, 20 bp, Artificial Sequence
  Description of Artificial Sequence; Note= Synthetic Construct
gtgtcaccagaagcaagcag

SEQ ID NO: 423 - DNA, 20 bp, Artificial Sequence
  Description of Artificial Sequence; Note= Synthetic Construct
gctgccaaatacccaaatgt

SEQ ID NO: 424 - DNA, 20 bp, Artificial Sequence
  Description of Artificial Sequence; Note= Synthetic Construct
tctgagagagagcaaagggc

SEQ ID NO: 425 - DNA, 21 bp, Artificial Sequence
  Description of Artificial Sequence; Note= Synthetic Construct
ttctggagatggatgttgttc

SEQ ID NO: 426 - DNA, 20 bp, Artificial Sequence
  Description of Artificial Sequence; Note= Synthetic Construct
tcccaactcagtttcaaccc

SEQ ID NO: 427 - DNA, 20 bp, Artificial Sequence
  Description of Artificial Sequence; Note= Synthetic Construct
ggtgctcagggactctcttg

SEQ ID NO: 428 - DNA, 20 bp, Artificial Sequence
  Description of Artificial Sequence; Note= Synthetic Construct
gcaggttgggttgaacttgt

SEQ ID NO: 429 - DNA, 20 bp, Artificial Sequence
  Description of Artificial Sequence; Note= Synthetic Construct
tgtcagtcagaaatgcagcc

SEQ ID NO: 430 - DNA, 20 bp, Artificial Sequence
  Description of Artificial Sequence; Note= Synthetic Construct
agggcggaagtagttccaat

SEQ ID NO: 431 - DNA, 20 bp, Artificial Sequence
  Description of Artificial Sequence; Note= Synthetic Construct
tgtcaccagtccctctcctc

SEQ ID NO: 432 - DNA, 20 bp, Artificial Sequence
  Description of Artificial Sequence; Note= Synthetic Construct
ctgagacaggcacagcactt

SEQ ID NO: 433 - DNA, 20 bp, Artificial Sequence
  Description of Artificial Sequence; Note= Synthetic Construct
atggaggtgcagtaggttgg

SEQ ID NO: 434 - DNA, 22 bp, Artificial Sequence
  Description of Artificial Sequence; Note= Synthetic Construct
gcttgaacaaattcaaattcca

SEQ ID NO: 435 - DNA, 20 bp, Artificial Sequence
  Description of Artificial Sequence; Note= Synthetic Construct
tcatccatcttcccattgct

SEQ ID NO: 436 - DNA, 20 bp, Artificial Sequence
  Description of Artificial Sequence; Note= Synthetic Construct
gcgtgtgcactgacacctat

SEQ ID NO: 437 - DNA, 20 bp, Artificial Sequence
  Description of Artificial Sequence; Note= Synthetic Construct
gaagccagtggtgagatggt

SEQ ID NO: 438 - DNA, 20 bp, Artificial Sequence
  Description of Artificial Sequence; Note= Synthetic Construct
cattcacagggaacatgtgg

SEQ ID NO: 439 - DNA, 20 bp, Artificial Sequence
  Description of Artificial Sequence; Note= Synthetic Construct
taaactgctaggcgtggagg

SEQ ID NO: 440 - DNA, 20 bp, Artificial Sequence
  Description of Artificial Sequence; Note= Synthetic Construct
tggatcattgcctgtgatgt

SEQ ID NO: 441 - DNA, 22 bp, Artificial Sequence
  Description of Artificial Sequence; Note= Synthetic Construct
ttctgagaacccaagggttaag

SEQ ID NO: 442 - DNA, 20 bp, Artificial Sequence
  Description of Artificial Sequence; Note= Synthetic Construct
cagcagccacttcctgagat

SEQ ID NO: 443 - DNA, 20 bp, Artificial Sequence
  Description of Artificial Sequence; Note= Synthetic Construct
tacagagagccccgtgagtt

SEQ ID NO: 444 - DNA, 20 bp, Artificial Sequence
  Description of Artificial Sequence; Note= Synthetic Construct
agccatcagccatattcagg

SEQ ID NO: 445 - DNA, 20 bp, Artificial Sequence
  Description of Artificial Sequence; Note= Synthetic Construct
catcaatgcatgtgggatgt

SEQ ID NO: 446 - DNA, 20 bp, Artificial Sequence
  Description of Artificial Sequence; Note= Synthetic Construct
gtctagtatcgggccaacca

SEQ ID NO: 447 - DNA, 20 bp, Artificial Sequence
  Description of Artificial Sequence; Note= Synthetic Construct
tgtgttgaattccctgcaac

SEQ ID NO: 448 - DNA, 20 bp, Artificial Sequence
  Description of Artificial Sequence; Note= Synthetic Construct
ggttcggagagctacggagt

SEQ ID NO: 449 - DNA, 20 bp, Artificial Sequence
  Description of Artificial Sequence; Note= Synthetic Construct
ttggatctggtttccactcc

SEQ ID NO: 450 - DNA, 20 bp, Artificial Sequence
  Description of Artificial Sequence; Note= Synthetic Construct
ctttctaagacgcccgtgag

SEQ ID NO: 451 - DNA, 20 bp, Artificial Sequence
  Description of Artificial Sequence; Note= Synthetic Construct
gaaagctggtctggactgga

SEQ ID NO: 452 - DNA, 20 bp, Artificial Sequence
  Description of Artificial Sequence; Note= Synthetic Construct
caactagcaggaggtggcat

SEQ ID NO: 453 - DNA, 20 bp, Artificial Sequence
  Description of Artificial Sequence; Note= Synthetic Construct
tctgcataggatgtggttgg

SEQ ID NO: 454 - DNA, 20 bp, Artificial Sequence
  Description of Artificial Sequence; Note= Synthetic Construct
gaaaggtctcggagtgctaa

SEQ ID NO: 455 - DNA, 20 bp, Artificial Sequence
  Description of Artificial Sequence; Note= Synthetic Construct
ttgtggacagtgtctggctc

SEQ ID NO: 456 - DNA, 20 bp, Artificial Sequence
  Description of Artificial Sequence; Note= Synthetic Construct
aggtcatgcactgtgagtcg

SEQ ID NO: 457 - DNA, 20 bp, Artificial Sequence
  Description of Artificial Sequence; Note= Synthetic Construct
ttagggcagagggtatgtgc

SEQ ID NO: 458 - DNA, 20 bp, Artificial Sequence
  Description of Artificial Sequence; Note= Synthetic Construct
atgacacctcaaggccagtc

SEQ ID NO: 459 - DNA, 20 bp, Artificial Sequence
  Description of Artificial Sequence; Note= Synthetic Construct
tggatgactacctgggcttc

SEQ ID NO: 460 - DNA, 20 bp, Artificial Sequence
  Description of Artificial Sequence; Note= Synthetic Construct
ggcaggaactcaatcctacg

SEQ ID NO: 461 - DNA, 20 bp, Artificial Sequence
  Description of Artificial Sequence; Note= Synthetic Construct
cgtaggattgagttcctgcc

SEQ ID NO: 462 - DNA, 20 bp, Artificial Sequence
  Description of Artificial Sequence; Note= Synthetic Construct
agtagtggcaccctggaatg

SEQ ID NO: 463 - DNA, 20 bp, Artificial Sequence
  Description of Artificial Sequence; Note= Synthetic Construct
ctgtttgcggccttctactc

SEQ ID NO: 464 - DNA, 20 bp, Artificial Sequence
  Description of Artificial Sequence; Note= Synthetic Construct
tctccttgcactggacacag

SEQ ID NO: 465 - DNA, 20 bp, Artificial Sequence
  Description of Artificial Sequence; Note= Synthetic Construct
gacagtccttggcctctcag

SEQ ID NO: 466 - DNA, 20 bp, Artificial Sequence
  Description of Artificial Sequence; Note= Synthetic Construct
ttaaccctgtggagagcaga

SEQ ID NO: 467 - DNA, 20 bp, Artificial Sequence
  Description of Artificial Sequence; Note= Synthetic Construct
ttctgggaagggttctgttg

SEQ ID NO: 468 - DNA, 20 bp, Artificial Sequence
  Description of Artificial Sequence; Note= Synthetic Construct
gagcagacgcttctcattcc

SEQ ID NO: 469 - DNA, 20 bp, Artificial Sequence
  Description of Artificial Sequence; Note= Synthetic Construct
agctgggagcagttgtcaat

SEQ ID NO: 470 - DNA, 20 bp, Artificial Sequence
  Description of Artificial Sequence; Note= Synthetic Construct
gcagctttggctctatgtcc

SEQ ID NO: 471 - DNA, 20 bp, Artificial Sequence
  Description of Artificial Sequence; Note= Synthetic Construct
ttcatgttgggttgttgtgg

SEQ ID NO: 472 - DNA, 20 bp, Artificial Sequence
  Description of Artificial Sequence; Note= Synthetic Construct
cagtcctgggagagttcagc

SEQ ID NO: 473 - DNA, 21 bp, Artificial Sequence
  Description of Artificial Sequence; Note= Synthetic Construct
aatcacttgaaagggtaggga

SEQ ID NO: 474 - DNA, 20 bp, Artificial Sequence
  Description of Artificial Sequence; Note= Synthetic Construct
cagtcctgggagagttcagc

SEQ ID NO: 475 - DNA, 20 bp, Artificial Sequence
  Description of Artificial Sequence; Note= Synthetic Construct
tcctcaaagacacccaggac

SEQ ID NO: 476 - DNA, 20 bp, Artificial Sequence
  Description of Artificial Sequence; Note= Synthetic Construct
atttggctgagatccctcct

SEQ ID NO: 477 - DNA, 20 bp, Artificial Sequence
  Description of Artificial Sequence; Note= Synthetic Construct
ttggttggcattcaactctg

SEQ ID NO: 478 - DNA, 20 bp, Artificial Sequence
  Description of Artificial Sequence; Note= Synthetic Construct
caggtctgcatctgtgccta

SEQ ID NO: 479 - DNA, 20 bp, Artificial Sequence
  Description of Artificial Sequence; Note= Synthetic Construct
ctccaggaggtggtagatgg

SEQ ID NO: 480 - DNA, 20 bp, Artificial Sequence
  Description of Artificial Sequence; Note= Synthetic Construct
gatctgtgggtgttcccagt

SEQ ID NO: 481 - DNA, 20 bp, Artificial Sequence
  Description of Artificial Sequence; Note= Synthetic Construct
gctgtggttgggaaatagga

SEQ ID NO: 482 - DNA, 20 bp, Artificial Sequence
  Description of Artificial Sequence; Note= Synthetic Construct
ctggatttcagagggagcag

SEQ ID NO: 483 - DNA, 20 bp, Artificial Sequence
  Description of Artificial Sequence; Note= Synthetic Construct
aagtggtccagtcttggtgc

SEQ ID NO: 484 - DNA, 20 bp, Artificial Sequence
  Description of Artificial Sequence; Note= Synthetic Construct
cgaaagccagatgtctccat

SEQ ID NO: 485 - DNA, 20 bp, Artificial Sequence
  Description of Artificial Sequence; Note= Synthetic Construct
atctgccataaccagcttcg

SEQ ID NO: 486 - DNA, 20 bp, Artificial Sequence
  Description of Artificial Sequence; Note= Synthetic Construct
agggacttgtctgctgtgct

SEQ ID NO: 487 - DNA, 20 bp, Artificial Sequence
  Description of Artificial Sequence; Note= Synthetic Construct
ctcacagacaccagcagctc

SEQ ID NO: 488 - DNA, 20 bp, Artificial Sequence
  Description of Artificial Sequence; Note= Synthetic Construct
cagcccatagcactctctcc

SEQ ID NO: 489 - DNA, 20 bp, Artificial Sequence
  Description of Artificial Sequence; Note= Synthetic Construct
gaggaagagtccatgtggga

SEQ ID NO: 490 - DNA, 20 bp, Artificial Sequence
  Description of Artificial Sequence; Note= Synthetic Construct
ccatggtttgcagcctctat

SEQ ID NO: 491 - DNA, 20 bp, Artificial Sequence
  Description of Artificial Sequence; Note= Synthetic Construct
gtttatgggtcgctgcatct

SEQ ID NO: 492 - DNA, 20 bp, Artificial Sequence
  Description of Artificial Sequence; Note= Synthetic Construct
gcagctgaacttggcatgta

SEQ ID NO: 493 - DNA, 20 bp, Artificial Sequence
  Description of Artificial Sequence; Note= Synthetic Construct
gcactggatgcattacctga

SEQ ID NO: 494 - DNA, 20 bp, Artificial Sequence
  Description of Artificial Sequence; Note= Synthetic Construct
gggctctccttcctgtctct

SEQ ID NO: 495 - DNA, 20 bp, Artificial Sequence
  Description of Artificial Sequence; Note= Synthetic Construct
ctttctggctcacaatgcaa

SEQ ID NO: 496 - DNA, 20 bp, Artificial Sequence
  Description of Artificial Sequence; Note= Synthetic Construct
cagacctgccttactctggc

SEQ ID NO: 497 - DNA, 20 bp, Artificial Sequence
  Description of Artificial Sequence; Note= Synthetic Construct
ctttctggctcacaatgcaa

SEQ ID NO: 498 - DNA, 20 bp, Artificial Sequence
  Description of Artificial Sequence; Note= Synthetic Construct
gcttctgttgacagccactg

SEQ ID NO: 499 - DNA, 20 bp, Artificial Sequence
  Description of Artificial Sequence; Note= Synthetic Construct
gcctagacctagtggccaga

SEQ ID NO: 500 - DNA, 20 bp, Artificial Sequence
  Description of Artificial Sequence; Note= Synthetic Construct
gggctccttgtcatcaatgt

SEQ ID NO: 501 - DNA, 20 bp, Artificial Sequence
  Description of Artificial Sequence; Note= Synthetic Construct
ggagagcttcctgtgtgacc

SEQ ID NO: 502 - DNA, 20 bp, Artificial Sequence
  Description of Artificial Sequence; Note= Synthetic Construct
agggtgacaacctggaacag

SEQ ID NO: 503 - DNA, 20 bp, Artificial Sequence
  Description of Artificial Sequence; Note= Synthetic Construct
aggtcaggatttgccacaac

SEQ ID NO: 504 - DNA, 20 bp, Artificial Sequence
  Description of Artificial Sequence; Note= Synthetic Construct
cacagaaacagggtttccca

SEQ ID NO: 505 - DNA, 25 bp, Artificial Sequence
  Description of Artificial Sequence; Note= Synthetic Construct
aacctgtgtcacttgcataattaaa

SEQ ID NO: 506 - DNA, 21 bp, Artificial Sequence
  Description of Artificial Sequence; Note= Synthetic Construct
gggtcaccagtgtaggtacga

SEQ ID NO: 507 - DNA, 21 bp, Artificial Sequence
  Description of Artificial Sequence; Note= Synthetic Construct
gaagacatacccaagacttgg

SEQ ID NO: 508 - DNA, 19 bp, Artificial Sequence
  Description of Artificial Sequence; Note= Synthetic Construct
acctgggactctgccatga

SEQ ID NO: 509 - DNA, 20 bp, Artificial Sequence
  Description of Artificial Sequence; Note= Synthetic Construct
cttgaagccttcctgatgct

SEQ ID NO: 510 - DNA, 24 bp, Artificial Sequence
  Description of Artificial Sequence; Note= Synthetic Construct
cctctagctcttgctacaaacaca

SEQ ID NO: 511 - DNA, 20 bp, Artificial Sequence
  Description of Artificial Sequence; Note= Synthetic Construct
aattctccctccatcccatc

SEQ ID NO: 512 - DNA, 20 bp, Artificial Sequence
  Description of Artificial Sequence; Note= Synthetic Construct
gtccagagctgaggagcaag

SEQ ID NO: 513 - DNA, 20 bp, Artificial Sequence
  Description of Artificial Sequence; Note= Synthetic Construct
acaggctgctgtccaagttt

SEQ ID NO: 514 - DNA, 20 bp, Artificial Sequence
  Description of Artificial Sequence; Note= Synthetic Construct
gcatctcagacacacggaga

SEQ ID NO: 515 - DNA, 20 bp, Artificial Sequence
  Description of Artificial Sequence; Note= Synthetic Construct
cctagcagggaggagctgta

SEQ ID NO: 516 - DNA, 20 bp, Artificial Sequence
  Description of Artificial Sequence; Note= Synthetic Construct
gcatcctcatggctcacttt

SEQ ID NO: 517 - DNA, 20 bp, Artificial Sequence
  Description of Artificial Sequence; Note= Synthetic Construct
aaagtgagccatgaggatgc

SEQ ID NO: 518 - DNA, 20 bp, Artificial Sequence
  Description of Artificial Sequence; Note= Synthetic Construct
tcttcaaagccaatcatcca

SEQ ID NO: 519 - DNA, 20 bp, Artificial Sequence
  Description of Artificial Sequence; Note= Synthetic Construct
ctgaacggtgctctttgaca

SEQ ID NO: 520 - DNA, 20 bp, Artificial Sequence
  Description of Artificial Sequence; Note= Synthetic Construct
ctttagaagccctggtgctg

SEQ ID NO: 521 - DNA, 20 bp, Artificial Sequence
  Description of Artificial Sequence; Note= Synthetic Construct
tcttaaggccttcccatcct

SEQ ID NO: 522 - DNA, 20 bp, Artificial Sequence
  Description of Artificial Sequence; Note= Synthetic Construct
aagcaactcccaatcctgtg

SEQ ID NO: 523 - DNA, 20 bp, Artificial Sequence
  Description of Artificial Sequence; Note= Synthetic Construct
tttcagcaggagacggaact

SEQ ID NO: 524 - DNA, 20 bp, Artificial Sequence
  Description of Artificial Sequence; Note= Synthetic Construct
ctgctctcacagatgagcgt

SEQ ID NO: 525 - DNA, 20 bp, Artificial Sequence
  Description of Artificial Sequence; Note= Synthetic Construct
taattgaagaggtgggtggc

SEQ ID NO: 526 - DNA, 21 bp, Artificial Sequence
  Description of Artificial Sequence; Note= Synthetic Construct
tgctttgcagacattggtaat

SEQ ID NO: 527 - DNA, 20 bp, Artificial Sequence
  Description of Artificial Sequence; Note= Synthetic Construct
gaaatgctcattgctgctga

SEQ ID NO: 528 - DNA, 20 bp, Artificial Sequence
  Description of Artificial Sequence; Note= Synthetic Construct
tccagcaaacacattcctga

SEQ ID NO: 529 - DNA, 20 bp, Artificial Sequence
  Description of Artificial Sequence; Note= Synthetic Construct
gagacccgtgagacaccagt

SEQ ID NO: 530 - DNA, 20 bp, Artificial Sequence
  Description of Artificial Sequence; Note= Synthetic Construct
ccaagtgaaaggaaacccaa

SEQ ID NO: 531 - DNA, 20 bp, Artificial Sequence
  Description of Artificial Sequence; Note= Synthetic Construct
gctctgtttccagagttggc

SEQ ID NO: 532 - DNA, 20 bp, Artificial Sequence
  Description of Artificial Sequence; Note= Synthetic Construct
aataggccaaagccagaggt

SEQ ID NO: 533 - DNA, 24 bp, Artificial Sequence
  Description of Artificial Sequence; Note= Synthetic Construct
tctcagatgacagaattactccaa

SEQ ID NO: 534 - DNA, 18 bp, Artificial Sequence
  Description of Artificial Sequence; Note= Synthetic Construct
cagagctgctgccaggat

SEQ ID NO: 535 - DNA, 19 bp, Artificial Sequence
  Description of Artificial Sequence; Note= Synthetic Construct
ctggccaacatggtgaaac

SEQ ID NO: 536 - DNA, 20 bp, Artificial Sequence
  Description of Artificial Sequence; Note= Synthetic Construct
gatgcatggcagagtgctaa

SEQ ID NO: 537 - DNA, 24 bp, Artificial Sequence
  Description of Artificial Sequence; Note= Synthetic Construct
cctgttgatcatattgtcaaggaa

SEQ ID NO: 538 - DNA, 20 bp, Artificial Sequence
  Description of Artificial Sequence; Note= Synthetic Construct
agggattagggagccagaga

SEQ ID NO: 539 - DNA, 20 bp, Artificial Sequence
  Description of Artificial Sequence; Note= Synthetic Construct
gcacccagtccagttagaga

SEQ ID NO: 540 - DNA, 20 bp, Artificial Sequence
  Description of Artificial Sequence; Note= Synthetic Construct
ttagagctgttgttgcctgg

SEQ ID NO: 541 - DNA, 20 bp, Artificial Sequence
  Description of Artificial Sequence; Note= Synthetic Construct
tcttgggtgggtcacttagc

SEQ ID NO: 542 - DNA, 21 bp, Artificial Sequence
  Description of Artificial Sequence; Note= Synthetic Construct
tcccttgagaaattcccagtc

SEQ ID NO: 543 - DNA, 20 bp, Artificial Sequence
  Description of Artificial Sequence; Note= Synthetic Construct
atggacagcttggaaggtca

SEQ ID NO: 544 - DNA, 20 bp, Artificial Sequence
  Description of Artificial Sequence; Note= Synthetic Construct
ctggttcttgcaccctcttc

SEQ ID NO: 545 - DNA, 20 bp, Artificial Sequence
  Description of Artificial Sequence; Note= Synthetic Construct
tggtcaggacagagccttct

SEQ ID NO: 546 - DNA, 20 bp, Artificial Sequence
  Description of Artificial Sequence; Note= Synthetic Construct
aaactgtgcaccaactgtgg

SEQ ID NO: 547 - DNA, 20 bp, Artificial Sequence
  Description of Artificial Sequence; Note= Synthetic Construct
agatggccaagccctaagtt

SEQ ID NO: 548 - DNA, 18 bp, Artificial Sequence
  Description of Artificial Sequence; Note= Synthetic Construct
cttccactcctggccctt

SEQ ID NO: 549 - DNA, 20 bp, Artificial Sequence
  Description of Artificial Sequence; Note= Synthetic Construct
tcaccagcccatttaaggag

SEQ ID NO: 550 - DNA, 20 bp, Artificial Sequence
  Description of Artificial Sequence; Note= Synthetic Construct
ctggaatagagtgtgtggcg

SEQ ID NO: 551 - DNA, 20 bp, Artificial Sequence
  Description of Artificial Sequence; Note= Synthetic Construct
tcagaagtgacagcgtttgc

SEQ ID NO: 552 - DNA, 20 bp, Artificial Sequence
  Description of Artificial Sequence; Note= Synthetic Construct
caagcagcatctgcattgtt

SEQ ID NO: 553 - DNA, 22 bp, Artificial Sequence
  Description of Artificial Sequence; Note= Synthetic Construct
ctccatctgaataaaggtagcg

SEQ ID NO: 554 - DNA, 20 bp, Artificial Sequence
  Description of Artificial Sequence; Note= Synthetic Construct
cgctccactgcctctctaat

SEQ ID NO: 555 - DNA, 20 bp, Artificial Sequence
  Description of Artificial Sequence; Note= Synthetic Construct
atactttcccagggaggacg

SEQ ID NO: 556 - DNA, 20 bp, Artificial Sequence
  Description of Artificial Sequence; Note= Synthetic Construct
gagtgtgcaaaggcatgtgt

SEQ ID NO: 557 - DNA, 20 bp, Artificial Sequence
  Description of Artificial Sequence; Note= Synthetic Construct
atttaagccttgggagtcgg

SEQ ID NO: 558 - DNA, 22 bp, Artificial Sequence
  Description of Artificial Sequence; Note= Synthetic Construct
gcctggaacatagtaggtgctc

SEQ ID NO: 559 - DNA, 21 bp, Artificial Sequence
  Description of Artificial Sequence; Note= Synthetic Construct
ctctgtccttggaagatgcac

SEQ ID NO: 560 - DNA, 21 bp, Artificial Sequence
  Description of Artificial Sequence; Note= Synthetic Construct
gaccctcttccatatttccca

SEQ ID NO: 561 - DNA, 21 bp, Artificial Sequence
  Description of Artificial Sequence; Note= Synthetic Construct
ccttgccatatgcagtaagag

SEQ ID NO: 562 - DNA, 20 bp, Artificial Sequence
  Description of Artificial Sequence; Note= Synthetic Construct
tagggctgttgtgaggaagg

SEQ ID NO: 563 - DNA, 20 bp, Artificial Sequence
  Description of Artificial Sequence; Note= Synthetic Construct
aggagggatggagtgggtat

SEQ ID NO: 564 - DNA, 20 bp, Artificial Sequence
  Description of Artificial Sequence; Note= Synthetic Construct
cctgccagtccactcctaga

SEQ ID NO: 565 - DNA, 19 bp, Artificial Sequence
  Description of Artificial Sequence; Note= Synthetic Construct
cgccatatctcctttggct

SEQ ID NO: 566 - DNA, 20 bp, Artificial Sequence
  Description of Artificial Sequence; Note= Synthetic Construct
gcacctcagctatcaggacc

SEQ ID NO: 567 - DNA, 20 bp, Artificial Sequence
  Description of Artificial Sequence; Note= Synthetic Construct
cacacaaatccacaagccct

SEQ ID NO: 568 - DNA, 20 bp, Artificial Sequence
  Description of Artificial Sequence; Note= Synthetic Construct
caccctgtatgttgccttgg

SEQ ID NO: 569 - DNA, 20 bp, Artificial Sequence
  Description of Artificial Sequence; Note= Synthetic Construct
aacacagccaggtggaattt

SEQ ID NO: 570 - DNA, 19 bp, Artificial Sequence
  Description of Artificial Sequence; Note= Synthetic Construct
caggcctgagagaagcaca

SEQ ID NO: 571 - DNA, 20 bp, Artificial Sequence
  Description of Artificial Sequence; Note= Synthetic Construct
tgttgggttgtaactgccct

SEQ ID NO: 572 - DNA, 19 bp, Artificial Sequence
  Description of Artificial Sequence; Note= Synthetic Construct
attcctgctcccaccgtct

SEQ ID NO: 573 - DNA, 20 bp, Artificial Sequence
  Description of Artificial Sequence; Note= Synthetic Construct
tagaccctccctccaaatcc

SEQ ID NO: 574 - DNA, 20 bp, Artificial Sequence
  Description of Artificial Sequence; Note= Synthetic Construct
gctggttgttgaggtggaat

SEQ ID NO: 575 - DNA, 20 bp, Artificial Sequence
  Description of Artificial Sequence; Note= Synthetic Construct
gagatgcgaaatgcagtcaa

SEQ ID NO: 576 - DNA, 19 bp, Artificial Sequence
  Description of Artificial Sequence; Note= Synthetic Construct
agcacaaagatgtgcaggc

SEQ ID NO: 577 - DNA, 22 bp, Artificial Sequence
  Description of Artificial Sequence; Note= Synthetic Construct
tgataatctccagtctgctcca

SEQ ID NO: 578 - DNA, 22 bp, Artificial Sequence
  Description of Artificial Sequence; Note= Synthetic Construct
gcagtggcttactgtttccttt

SEQ ID NO: 579 - DNA, 20 bp, Artificial Sequence
  Description of Artificial Sequence; Note= Synthetic Construct
caggacacatgcacttgagg

SEQ ID NO: 580 - DNA, 20 bp, Artificial Sequence
  Description of Artificial Sequence; Note= Synthetic Construct
actttcctccacatggcaaa

SEQ ID NO: 581 - DNA, 20 bp, Artificial Sequence
  Description of Artificial Sequence; Note= Synthetic Construct
acagagtgctgtgtgttggg

SEQ ID NO: 582 - DNA, 20 bp, Artificial Sequence
  Description of Artificial Sequence; Note= Synthetic Construct
gacactggagcgaaatgtca

SEQ ID NO: 583 - DNA, 20 bp, Artificial Sequence
  Description of Artificial Sequence; Note= Synthetic Construct
ttgcatgacccatgactacc

SEQ ID NO: 584 - DNA, 20 bp, Artificial Sequence
  Description of Artificial Sequence; Note= Synthetic Construct
cttcccaactccctggtcac

SEQ ID NO: 585 - DNA, 20 bp, Artificial Sequence
  Description of Artificial Sequence; Note= Synthetic Construct
gagcctttctcctgtccaag

SEQ ID NO: 586 - DNA, 20 bp, Artificial Sequence
  Description of Artificial Sequence; Note= Synthetic Construct
ctaggtgctctccagggttg

SEQ ID NO: 587 - DNA, 20 bp, Artificial Sequence
  Description of Artificial Sequence; Note= Synthetic Construct
ttaaggagagtcagcctggg

SEQ ID NO: 588 - DNA, 20 bp, Artificial Sequence
  Description of Artificial Sequence; Note= Synthetic Construct
caagagagtccctgagcacc

SEQ ID NO: 589 - DNA, 20 bp, Artificial Sequence
  Description of Artificial Sequence; Note= Synthetic Construct
gggttgaaactgagttggga

SEQ ID NO: 590 - DNA, 20 bp, Artificial Sequence
  Description of Artificial Sequence; Note= Synthetic Construct
ggaagctcagctgtacccat

SEQ ID NO: 591 - DNA, 20 bp, Artificial Sequence
  Description of Artificial Sequence; Note= Synthetic Construct
ttcccatgcccaagtatttc

SEQ ID NO: 592 - DNA, 18 bp, Artificial Sequence
  Description of Artificial Sequence; Note= Synthetic Construct
cctctgcccttcccatct

SEQ ID NO: 593 - DNA, 20 bp, Artificial Sequence
  Description of Artificial Sequence; Note= Synthetic Construct
gcctaaggtcacacagctcc

SEQ ID NO: 594 - DNA, 19 bp, Artificial Sequence
  Description of Artificial Sequence; Note= Synthetic Construct
cacattactccctgcaccg

SEQ ID NO: 595 - DNA, 20 bp, Artificial Sequence
  Description of Artificial Sequence; Note= Synthetic Construct
gactgccctcaagtttcagc

SEQ ID NO: 596 - DNA, 20 bp, Artificial Sequence
  Description of Artificial Sequence; Note= Synthetic Construct
aactccctgtttggcatctg

SEQ ID NO: 597 - DNA, 20 bp, Artificial Sequence
  Description of Artificial Sequence; Note= Synthetic Construct
cagcctggcagctcttctat

SEQ ID NO: 598 - DNA, 20 bp, Artificial Sequence
  Description of Artificial Sequence; Note= Synthetic Construct
atagggtgacagggatgtgg

SEQ ID NO: 599 - DNA, 20 bp, Artificial Sequence
  Description of Artificial Sequence; Note= Synthetic Construct
tctgtgggactggagaaagg

SEQ ID NO: 600 - DNA, 20 bp, Artificial Sequence
  Description of Artificial Sequence; Note= Synthetic Construct
ttctgtgacccgtagagcct

SEQ ID NO: 601 - DNA, 20 bp, Artificial Sequence
  Description of Artificial Sequence; Note= Synthetic Construct
tatgccgtgtagggattgtg

SEQ ID NO: 602 - DNA, 19 bp, Artificial Sequence
  Description of Artificial Sequence; Note= Synthetic Construct
agagggcttggcgttgttc

SEQ ID NO: 603 - DNA, 20 bp, Artificial Sequence
  Description of Artificial Sequence; Note= Synthetic Construct
ctcccaaagtgctgggatta

SEQ ID NO: 604 - DNA, 20 bp, Artificial Sequence
  Description of Artificial Sequence; Note= Synthetic Construct
gcttgtcacccaaatgacct

SEQ ID NO: 605 - DNA, 20 bp, Artificial Sequence
  Description of Artificial Sequence; Note= Synthetic Construct
cagcctcttacaggcgtttc

SEQ ID NO: 606 - DNA, 20 bp, Artificial Sequence
  Description of Artificial Sequence; Note= Synthetic Construct
cagagggatgtgcaatgaga

SEQ ID NO: 607 - DNA, 20 bp, Artificial Sequence
  Description of Artificial Sequence; Note= Synthetic Construct
actggagatgttcctcgcac

SEQ ID NO: 608 - DNA, 20 bp, Artificial Sequence
  Description of Artificial Sequence; Note= Synthetic Construct
aggacattggaatggagctg

SEQ ID NO: 609 - DNA, 20 bp, Artificial Sequence
  Description of Artificial Sequence; Note= Synthetic Construct
agctgtttgggactggtgac

SEQ ID NO: 610 - DNA, 20 bp, Artificial Sequence
  Description of Artificial Sequence; Note= Synthetic Construct
cagacctgtccacattcgtg

SEQ ID NO: 611 - DNA, 20 bp, Artificial Sequence
  Description of Artificial Sequence; Note= Synthetic Construct
gtagaagggctgtggcattc

SEQ ID NO: 612 - DNA, 20 bp, Artificial Sequence
  Description of Artificial Sequence; Note= Synthetic Construct
cgccctaaagactccaagac

SEQ ID NO: 613 - DNA, 21 bp, Artificial Sequence
  Description of Artificial Sequence; Note= Synthetic Construct
ccctgtgtcttctagctgtgc

SEQ ID NO: 614 - DNA, 20 bp, Artificial Sequence
  Description of Artificial Sequence; Note= Synthetic Construct
ctgccctcagagatgattcc

SEQ ID NO: 615 - DNA, 20 bp, Artificial Sequence
  Description of Artificial Sequence; Note= Synthetic Construct
gcgtctgtagagatccaggc

SEQ ID NO: 616 - DNA, 20 bp, Artificial Sequence
  Description of Artificial Sequence; Note= Synthetic Construct
ggcatatcccacaatccaag

SEQ ID NO: 617 - DNA, 20 bp, Artificial Sequence
  Description of Artificial Sequence; Note= Synthetic Construct
cggaacacacagagtgatgg

SEQ ID NO: 618 - DNA, 20 bp, Artificial Sequence
  Description of Artificial Sequence; Note= Synthetic Construct
tctaactcgagcatcagccc

SEQ ID NO: 619 - DNA, 20 bp, Artificial Sequence
  Description of Artificial Sequence; Note= Synthetic Construct
ttctttgcatctccaagcct

SEQ ID NO: 620 - DNA, 20 bp, Artificial Sequence
  Description of Artificial Sequence; Note= Synthetic Construct
catggaaggatcagactggc

SEQ ID NO: 621 - DNA, 20 bp, Artificial Sequence
  Description of Artificial Sequence; Note= Synthetic Construct
catctgggtggcttgtcata

SEQ ID NO: 622 - DNA, 20 bp, Artificial Sequence
  Description of Artificial Sequence; Note= Synthetic Construct
gaagcagggcaagtgttgat

SEQ ID NO: 623 - DNA, 20 bp, Artificial Sequence
  Description of Artificial Sequence; Note= Synthetic Construct
atgctgtttctttcttgggc

SEQ ID NO: 624 - DNA, 20 bp, Artificial Sequence
  Description of Artificial Sequence; Note= Synthetic Construct
aatgatcaggatgggtcagg

SEQ ID NO: 625 - DNA, 20 bp, Artificial Sequence
  Description of Artificial Sequence; Note= Synthetic Construct
cactagggaacacgggtacg

SEQ ID NO: 626 - DNA, 20 bp, Artificial Sequence
  Description of Artificial Sequence; Note= Synthetic Construct
tctgtgtcccactgcacact

SEQ ID NO: 627 - DNA, 20 bp, Artificial Sequence
  Description of Artificial Sequence; Note= Synthetic Construct
agactggatgtatttgggcg

SEQ ID NO: 628 - DNA, 20 bp, Artificial Sequence
  Description of Artificial Sequence; Note= Synthetic Construct
gctgctgcagggagatttat

SEQ ID NO: 629 - DNA, 20 bp, Artificial Sequence
  Description of Artificial Sequence; Note= Synthetic Construct
agatggctgtgtgtgtggag

SEQ ID NO: 630 - DNA, 20 bp, Artificial Sequence
  Description of Artificial Sequence; Note= Synthetic Construct
ttccttctgcaaattggtcc

SEQ ID NO: 631 - DNA, 19 bp, Artificial Sequence
  Description of Artificial Sequence; Note= Synthetic Construct
gccactcaagccagacact

SEQ ID NO: 632 - DNA, 20 bp, Artificial Sequence
  Description of Artificial Sequence; Note= Synthetic Construct
tgattccggctcaaacctac

SEQ ID NO: 633 - DNA, 20 bp, Artificial Sequence
  Description of Artificial Sequence; Note= Synthetic Construct
ggaatgatgtagcctttgcc

SEQ ID NO: 634 - DNA, 20 bp, Artificial Sequence
  Description of Artificial Sequence; Note= Synthetic Construct
ttgggtagcttgatcttgcc

SEQ ID NO: 635 - DNA, 20 bp, Artificial Sequence
  Description of Artificial Sequence; Note= Synthetic Construct
gatacgggtcaagctgtggt

SEQ ID NO: 636 - DNA, 20 bp, Artificial Sequence
  Description of Artificial Sequence; Note= Synthetic Construct
cagtcctgggagagttcagc

SEQ ID NO: 637 - DNA, 20 bp, Artificial Sequence
  Description of Artificial Sequence; Note= Synthetic Construct
tctcggagtgtccctaggtc

SEQ ID NO: 638 - DNA, 20 bp, Artificial Sequence
  Description of Artificial Sequence; Note= Synthetic Construct
ggcaagcaatgagaggagac

SEQ ID NO: 639 - DNA, 20 bp, Artificial Sequence
  Description of Artificial Sequence; Note= Synthetic Construct
tacctccggagacttcatgc

SEQ ID NO: 640 - DNA, 20 bp, Artificial Sequence
  Description of Artificial Sequence; Note= Synthetic Construct
ctcctgggaccatctctgaa

SEQ ID NO: 641 - DNA, 20 bp, Artificial Sequence
  Description of Artificial Sequence; Note= Synthetic Construct
cccttcactgggctatttca

SEQ ID NO: 642 - DNA, 20 bp, Artificial Sequence
  Description of Artificial Sequence; Note= Synthetic Construct
atctttgggtatgctgggtg

SEQ ID NO: 643 - DNA, 19 bp, Artificial Sequence
  Description of Artificial Sequence; Note= Synthetic Construct
ttcctgtggctgcagaaag

SEQ ID NO: 644 - DNA, 20 bp, Artificial Sequence
  Description of Artificial Sequence; Note= Synthetic Construct
agcaagtgtttcagtgccaa

SEQ ID NO: 645 - DNA, 20 bp, Artificial Sequence
  Description of Artificial Sequence; Note= Synthetic Construct
ttccgttctgactcatctgg

SEQ ID NO: 646 - DNA, 20 bp, Artificial Sequence
  Description of Artificial Sequence; Note= Synthetic Construct
gggccttaaatgcctgatct

SEQ ID NO: 647 - DNA, 20 bp, Artificial Sequence
  Description of Artificial Sequence; Note= Synthetic Construct
tgtggctgtcccattgtcta

SEQ ID NO: 648 - DNA, 20 bp, Artificial Sequence
  Description of Artificial Sequence; Note= Synthetic Construct
tcagcgaagcctgatcctac

SEQ ID NO: 649 - DNA, 18 bp, Artificial Sequence
  Description of Artificial Sequence; Note= Synthetic Construct
aggacccaggctccatgt

SEQ ID NO: 650 - DNA, 20 bp, Artificial Sequence
  Description of Artificial Sequence; Note= Synthetic Construct
gcatctgtgctagcaatcca

SEQ ID NO: 651 - DNA, 19 bp, Artificial Sequence
  Description of Artificial Sequence; Note= Synthetic Construct
gtcaccacaggctgctcac

SEQ ID NO: 652 - DNA, 20 bp, Artificial Sequence
  Description of Artificial Sequence; Note= Synthetic Construct
aaccacgtcaggagatgacc

SEQ ID NO: 653 - DNA, 20 bp, Artificial Sequence
  Description of Artificial Sequence; Note= Synthetic Construct
tgggttggacctgtaccttc

SEQ ID NO: 654 - DNA, 20 bp, Artificial Sequence
  Description of Artificial Sequence; Note= Synthetic Construct
tccttccatctgggattctg

SEQ ID NO: 655 - DNA, 20 bp, Artificial Sequence
  Description of Artificial Sequence; Note= Synthetic Construct
gcactgacatccatcacacc

SEQ ID NO: 656 - DNA, 20 bp, Artificial Sequence
  Description of Artificial Sequence; Note= Synthetic Construct
ttgtctgggtgaaatctggc

SEQ ID NO: 657 - DNA, 20 bp, Artificial Sequence
  Description of Artificial Sequence; Note= Synthetic Construct
ggtgctggaattgtgatcct

SEQ ID NO: 658 - DNA, 20 bp, Artificial Sequence
  Description of Artificial Sequence; Note= Synthetic Construct
gcagatgtcaaagttggggt

SEQ ID NO: 659 - DNA, 20 bp, Artificial Sequence
  Description of Artificial Sequence; Note= Synthetic Construct
gagggaggccaacatctaca

SEQ ID NO: 660 - DNA, 20 bp, Artificial Sequence
  Description of Artificial Sequence; Note= Synthetic Construct
ctgaacccttccagtgagga

SEQ ID NO: 661 - DNA, 20 bp, Artificial Sequence
  Description of Artificial Sequence; Note= Synthetic Construct
tgaacaggatgcatttggaa

SEQ ID NO: 662 - DNA, 20 bp, Artificial Sequence
  Description of Artificial Sequence; Note= Synthetic Construct
cctaaggaaggtctccaccc

SEQ ID NO: 663 - DNA, 20 bp, Artificial Sequence
  Description of Artificial Sequence; Note= Synthetic Construct
agagagggcagggagacaat

SEQ ID NO: 664 - DNA, 20 bp, Artificial Sequence
  Description of Artificial Sequence; Note= Synthetic Construct
ggattgagtcttgcccagat

SEQ ID NO: 665 - DNA, 20 bp, Artificial Sequence
  Description of Artificial Sequence; Note= Synthetic Construct
ccaaccaaatgctgaaacct

SEQ ID NO: 666 - DNA, 20 bp, Artificial Sequence
  Description of Artificial Sequence; Note= Synthetic Construct
gttatcccagcccacacttg

SEQ ID NO: 667 - DNA, 20 bp, Artificial Sequence
  Description of Artificial Sequence; Note= Synthetic Construct
gttcctttctggctccctct

SEQ ID NO: 668 - DNA, 20 bp, Artificial Sequence
  Description of Artificial Sequence; Note= Synthetic Construct
aacaccatcccatcaccagt

SEQ ID NO: 669 - DNA, 20 bp, Artificial Sequence
  Description of Artificial Sequence; Note= Synthetic Construct
cacgagaatagcatgggaaa

SEQ ID NO: 670 - DNA, 20 bp, Artificial Sequence
  Description of Artificial Sequence; Note= Synthetic Construct
tactgacactggccttccct

SEQ ID NO: 671 - DNA, 20 bp, Artificial Sequence
  Description of Artificial Sequence; Note= Synthetic Construct
tgtttctgataagggcctgg

SEQ ID NO: 672 - DNA, 20 bp, Artificial Sequence
  Description of Artificial Sequence; Note= Synthetic Construct
ggagcttctgttgggatcaa

SEQ ID NO: 673 - DNA, 20 bp, Artificial Sequence
  Description of Artificial Sequence; Note= Synthetic Construct
acactcaggcccagtacagc

SEQ ID NO: 674 - DNA, 20 bp, Artificial Sequence
  Description of Artificial Sequence; Note= Synthetic Construct
tgtgatgagccaggttcttg

SEQ ID NO: 675 - DNA, 20 bp, Artificial Sequence
  Description of Artificial Sequence; Note= Synthetic Construct
tgagcctccatttctccatc

SEQ ID NO: 676 - DNA, 20 bp, Artificial Sequence
  Description of Artificial Sequence; Note= Synthetic Construct
cagtggcatcacaggtcagt

SEQ ID NO: 677 - DNA, 20 bp, Artificial Sequence
  Description of Artificial Sequence; Note= Synthetic Construct
aagcctggagctagtggaca

SEQ ID NO: 678 - DNA, 20 bp, Artificial Sequence
  Description of Artificial Sequence; Note= Synthetic Construct
cagaggaagccaggacctaa

SEQ ID NO: 679 - DNA, 20 bp, Artificial Sequence
  Description of Artificial Sequence; Note= Synthetic Construct
atctctgagaagcccaccct

SEQ ID NO: 680 - DNA, 20 bp, Artificial Sequence
  Description of Artificial Sequence; Note= Synthetic Construct
gaagccaagaagcagactgg

SEQ ID NO: 681 - DNA, 20 bp, Artificial Sequence
  Description of Artificial Sequence; Note= Synthetic Construct
agagccagaaggtgacttgc

SEQ ID NO: 682 - DNA, 20 bp, Artificial Sequence
  Description of Artificial Sequence; Note= Synthetic Construct
caacccaaagttcagtgcag

SEQ ID NO: 683 - DNA, 20 bp, Artificial Sequence
  Description of Artificial Sequence; Note= Synthetic Construct
tgcactgaactttgggttga

SEQ ID NO: 684 - DNA, 20 bp, Artificial Sequence
  Description of Artificial Sequence; Note= Synthetic Construct
agacagcagtggtggtgaca

SEQ ID NO: 685 - DNA, 20 bp, Artificial Sequence
  Description of Artificial Sequence; Note= Synthetic Construct
ttgggaggattaatggagcc

SEQ ID NO: 686 - DNA, 20 bp, Artificial Sequence
  Description of Artificial Sequence; Note= Synthetic Construct
acctctactgacaggcccac

SEQ ID NO: 687 - DNA, 20 bp, Artificial Sequence
  Description of Artificial Sequence; Note= Synthetic Construct
gatggaatgggagacaatgg

SEQ ID NO: 688 - DNA, 20 bp, Artificial Sequence
  Description of Artificial Sequence; Note= Synthetic Construct
gggaggaaagagggagaatg

SEQ ID NO: 689 - DNA, 21 bp, Artificial Sequence
  Description of Artificial Sequence; Note= Synthetic Construct
gctatgatgcatgcaaatgtt

SEQ ID NO: 690 - DNA, 20 bp, Artificial Sequence
  Description of Artificial Sequence; Note= Synthetic Construct
ctgcatcttgaattcgctga

SEQ ID NO: 691 - DNA, 19 bp, Artificial Sequence
  Description of Artificial Sequence; Note= Synthetic Construct
cagcacccagaagaggagg

SEQ ID NO: 692 - DNA, 20 bp, Artificial Sequence
  Description of Artificial Sequence; Note= Synthetic Construct
ggactaagagcctccaaggg

SEQ ID NO: 693 - DNA, 19 bp, Artificial Sequence
  Description of Artificial Sequence; Note= Synthetic Construct
gtcctctcccagcctcttg

SEQ ID NO: 694 - DNA, 20 bp, Artificial Sequence
  Description of Artificial Sequence; Note= Synthetic Construct
actgcttctcagctgcctct

(19) United States
(12) Patent Application Publication
(10) Pub. No.: US 2006/0223062 A1
(43) Pub. Date: Oct. 5, 2006

(54) Rapid direct sequence analysis of multi-exon genes

(76) Inventors: Kevin M. Flanigan, Salt Lake City, UT (US); Robert B. Weiss, Salt Lake City, UT (US); Diane M. Dunn, Salt Lake City, UT (US); Andrew Von Niederhausern, Salt Lake City, UT (US)

Correspondence Address: NEEDLE & ROSENBERG, P.C., SUITE 1000, 999 PEACHTREE STREET, ATLANTA, GA, 30309-3915, US

(21) Appl. No.: 10/539,178

(22) PCT Filed: Dec. 17, 2003
(86) PCT No.: PCT/US03/40278

§ 371 Date: Jan. 23, 2006

Related U.S. Application Data
(60) Provisional application No. 60/433,774, filed on Dec. 17, 2002.

(51) Int. Cl. C12Q 1/68; C12P 19/34

(52) U.S. Cl. 435/6; 435/91.2

(57) ABSTRACT

Disclosed is a Single Condition Amplification/Internal Primer (SCAIP) sequencing method which allows for the rapid, accurate, and economical analysis of any large multi-exon gene. The method can be used to detect genomic mutations in any large multi-exon gene including the dystrophin gene. In some forms, the method can rely on amplification of a large number of exons at a single set of PCR temperatures with a first set of amplification primers followed by sequencing without optimization of individual amplicon conditions, using a second, internal set of sequencing primers. The SCAIP method provides for the identification and analysis of specific individual genomic mutations such as deletions, point mutations, frameshifts, or combinations thereof, in gene complexes with multiple exons/introns spanning large genomic regions.

### CROSS-REFERENCE TO RELATED APPLICATIONS

This application claims benefit of U.S. Provisional Application No. 60/433,774, filed Dec. 17, 2002. Application Ser. No. 60/433,774, filed Dec. 17, 2002, is hereby incorporated herein by reference in its entirety.

### STATEMENT REGARDING FEDERALLY SPONSORED RESEARCH

The research described herein was supported by the Parent Project Muscular Dystrophy, the Muscular Dystrophy Association, the Primary Children's Research Foundation and the National Institutes of Health (NIH R01 NS43264-01 and NIH U01 HG02138-04). The U.S. Government has certain rights in this invention.

### FIELD

The compositions, materials, methods, and devices disclosed herein relate to a Single Condition Amplification/Internal Primer (SCAIP) sequencing method for direct sequence analysis of large multi-exon genes from genomic DNA samples and identifying mutations in multi-exon genes. Also, disclosed are methods for diagnosing dystrophinopathies in patients. The disclosed compositions, materials, methods, and devices further relate to compositions for PCR primer sets and sequencing primer sets recognizing the exons or proximal promoter regions for the dystrophin gene.

### BACKGROUND

The dystrophinopathies, Duchenne Muscular Dystrophy (MD) and Becker Muscular Dystrophy (BMD), are the most common inherited disorders of muscle. The prevalence of DMD is generally estimated at 1:3500 live male births (Emery (1991) Neuromuscul Disord 1: 19-29). The dystrophin gene is located at Xp21 and is comprised of 79 exons and 8 tissue-specific promoters distributed across approximately 2.2 million base pairs of genomic sequence, making dystrophin the largest gene yet described. Both DMD and BMD are due to mutations in the dystrophin gene. Dystrophin gene deletions are found in approximately 55% of Becker and 65% of Duchenne patients; point mutations account for around 30% of mutations and duplications account for the remainder (Miller et al. (1994) Neurol Clin 12:699-725).

Genetic testing for deletions has relied upon a multiplex PCR technique with amplification of fragments containing 18 to 25 of the 79 exons for the gene (Beggs et al. (1990) Hum Genet 86:45-48; Chamberlain et al. (1990) Multiplex PCR for the diagnosis of Duchenne muscular dystrophy. In: Innis et al. (eds) PCR Protocols: A Guide to Methods and Applications. Academic Press, San Francisco, pp. 272-281) and deletions detected as absent or size-shifted bands on agarose gel analysis. Deletions tend to occur in “hotspots” within the dystrophin gene, and it is estimated that 98% of all dystrophin deletions are detectable by this method.

Testing for dystrophin point mutations has only been available on a research basis from specialized laboratories. Such analysis requires sequencing of all 79 exons and eight aromoters. There are no particularly common point mutations or point mutation hotspots currently known, and each affected family may carry a unique mutation in this enormous gene (so-called “private mutations” as they are exclusive to individual families). Instead of direct sequence analysis, some research laboratories perform point mutation analysis on cDNA derived by reverse transcription-PCR (RT-PCR) from muscle mRNA. As an alternative, other laboratories have utilized the protein truncation test (PTT), which may be performed using peripheral blood lymphocyte DNA (Roest et al. (1993) Neuromuscul Disord 3:391-394) but often uses mRNA derived from muscle biopsy (Tuffery-Giraud et al. (1999) Hum Mutat 14:359-368). There is a drawback to approaches that require muscle biopsy, an invasive procedure with a generally accepted risk of complications (bleeding, infections, hematoma formation) of around 1%, and one that may often be associated with psychological distress for children.

Direct sequence analysis of the dystrophin gene has been considered too labor-intensive, expensive, and time-consuming (Bennett et al. (2001) BMC Genet 2:17), but several groups have recently developed strategies to detect exonic sequence variations by screening methods, followed by direct sequence analysis of only variant fragments. One of these strategies is based on single-strand conformational polymorphism (SSCP) analysis (Mendell et al. (2001) Neurology 57:645-650). This strategy relies on multiplexing up to 23 amplicons per lane with SSCP in up to five conditions. Mendell et al. report that up to 75% of non-deletion mutations may be detected by this method, but there are several drawbacks. One is that all band variations detected by SSCP techniques still need to be sequenced to determine whether they represent pathogenic mutations; the dystrophin gene, because of its size, has many reported polymorphisms. Another problem is that for economies of scale in reagents and technician time, individual samples may need to be saved until multiple samples are available for simultaneous analysis of band variation.

A second screening method relies upon denaturing high-performance liquid chromatography (DHPLC) (Bennett et al. (2001) BMC Genet 2:17). This strategy screens for DNA variations by separating heteroduplex and homoduplex DNA fragments by reverse phase liquid chromatography followed by direct sequence analysis of variant amplicons. Using this method, Bennett et al. detected point mutations in 6/8 DNA samples from patients without deletions, and argued for its use on an economic as well as scientific basis (Bennett et al. (2001) BMC Genet 2:17). Another screening strategy includes double gradient, denaturing gradient gel electrophoresis (DGGE) (Cremonesi et al. (1997) Biotechniques 22:326-330). A drawback to each of these prior art screening methods is the lack of sensitivity. While each method can detect both mutations and non-disease-associated polymorphisms, an additional sequencing step is required to distinguish between these possibilities.

Therefore, in light of the difficulties and short-comings with detecting and characterizing mutations in large multi-exon genes, such as the dystrophin gene, there exists a need for rapid, accurate, and economical sequence analysis of such genes. Disclosed herein are compositions, materials, methods, and devices that satisfy this need.

### SUMMARY

In accordance with the purposes of the disclosed compositions, materials, methods, and devices, as embodied and broadly described herein, the disclosed subject matter, in one aspect, relates to a Single Condition Amplification/Internal Primer (SCAIP) sequencing method which allows for the rapid, accurate, and economical analysis of any large multi-exon gene.

An additional aspect of this method is to detect genomic mutations in any large, multi-exon gene including the dystrophin gene.

In accomplishing this and other objects, there has been provided, according to one aspect of the disclosed method, a method relying on amplification of a large number of exons at a single set of PCR temperatures with a first set of amplification primers followed by sequencing without optimization of individual amplicon conditions, using a second, internal set of sequencing primers. The SCAIP sequencing method comprises the steps of:
providing a PCR reaction plate wherein the wells of each plate contain genomic DNA;
adding to each of the wells a different set of left and right PCR primers complementary to a single exonic region or proximal promoter segment for a multi-exon gene of interest and performing a PCR reaction at a uniform set of temperatures;
purifying PCR fragments for the single exonic region or the proximal promoter segment from each of the wells, adding the fragments to a well of a cycle sequencing reaction plate to which is added left and/or right internal sequencing primers corresponding to the single exonic regions or the proximal promoter fragments and sequencing at a uniform set of temperatures;
purification of sequencing products followed by electrophoretic separation and fluorescent detection of nucleotides on a sequence analyzer; and
nucleotide sequence characterization.

More generally, some forms of the disclosed methods involve amplification of a large number of amplicons from a gene or nucleic acid region of interest under the same reaction conditions with a first set of amplification primers followed by sequencing under the same reaction conditions using a second, internal set of sequencing primers. The amplification reactions are preferable carried out simultaneously and/or on the same solid support. The sequencing reactions can be carried out simultaneously and/or on the same solid support. The amplification and sequencing reactions can be carried out on the same solid support (for example, without transfer of amplification products to a different solid support or to different reaction chambers) or different solid supports. Purification of the amplification products prior to sequencing is preferred but not required. The general method can comprise the steps of:
adding to each of a plurality of reaction chambers a nucleic acid sample and a different set of amplification primers, wherein each set of amplification primers is complementary to a single amplicon segment of a gene or nucleic acid region of interest (such as an exonic region or proximal promoter segment of a multi-exon gene of interest) and performing an amplification reaction for each reaction chamber under the same reaction conditions;
bringing into contact in each of a plurality of reaction chambers an amplicon from a different one of the amplification reactions and one or more sequencing primers corresponding to the amplicon and performing a sequencing reaction for each reaction chamber under the same reaction conditions; and
analyzing the sequences of the amplicons.

The nucleic acid sample generally will be the same for each of the reaction chambers in a set of reactions for the analysis of a gene or nucleic acid region of interest. Each reaction chamber is used to amplify and/or sequence a different amplicon from the gene or nucleic acid region of interest. Useful forms of the method involve amplifying and sequencing all relevant amplicons in the gene or nucleic acid region of interest.

Pursuant to another aspect, the disclosed methods provide for a method of diagnosing mutations in a large multi-exon gene. Individuals may also be tested using the method to identify their status as carriers of DMD or BMD.

Another aspect of the disclosed methods and compositions is the specific amplifying and sequencing primers for the dystrophin gene and their use in a detection kit for DMD or BMD mutations.

Additional advantages of the disclosed methods and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is an agarose gel analysis of primary PCR products from a multi-exon deletion case missing exons 20 to 30 and the DMD260 promoter.

FIG. 2 is a graph of the average Phrap score coverage of DMD exons and promoter regions.

### DETAILED DESCRIPTION

The compositions, materials, methods, and devices described herein may be understood more readily by reference to the following detailed description of specific aspects of the disclosed subject matter, and methods and the Examples included therein and to the Figures and their previous and following description.

Also, throughout this specification, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Before the present compositions, materials, methods, and devices, are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Disclosed herein are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an internal primer is disclosed and discussed and a number of modifications that can be made to a number of molecules including the internal primer are discussed, each and every combination and permutation of the internal primer and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C, D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. General Definitions:

In this specification and in the claims that follow, reference will be made to a number of terms, which shall be defined to have the following meanings:

As used in the specification and the appended claims, the singular forms “a,” “an,” and “the” include plural referents unless the context clearly dictates otherwise. Thus, for example, reference to “a nucleotide” includes mixtures of two or more such nucleotides, reference to “an amino acid” includes mixtures of two or more such amino acids, reference to “the primer” includes mixtures of two or more such primers, and the like.

“Optional” or “optionally” means that the subsequently described event or circumstance can or cannot occur, and that the description includes instances where the event or circumstance occurs and instances where it does not. For example, the phrase “amplicons can optionally be purified” means that the amplicons may or may not be purified and that the description includes both methods where the amplicons are purified and methods where the amplicons are not purified.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Individual,” as used herein, means a subject. In one aspect, the individual is a mammal such as a primate, and, in another aspect, the individual is a human. The term “individual” also includes domesticated animals (e.g., cats, dogs, etc.), livestock (e.g., cattle, horses, pigs, sheep, goats, etc.), and laboratory animals (e.g., mouse, rabbit, rat, guinea pig, etc.).

There are a variety of molecules disclosed herein that are nucleic acid based, including for example the nucleic acids that encode, for example, dystrophin as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar of phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86,6553-6556).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

There are a variety of sequences related to, for example, the dystrophin gene as well as any other nucleic acids sequences that are disclosed on GenBank, and these sequences and others are herein incorporated by reference in their entireties as well as for individual subsequences contained therein.

A variety of sequences are provided herein and these and others can be found in GenBank, at www.ncbi.nlm.nih.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

Disclosed are compositions including primers and probes, which are capable of interacting with the genes disclosed herein. In certain embodiments the primers are used to support DNA amplification reactions. In other embodiments, the primers are used to support sequencing reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

B. Method:

Disclosed herein is a Single Condition Amplification/Internal Primer (SCAIP) sequencing method which allows for the rapid, accurate, and economical analysis of any large multi-exon gene. This method is particularly useful for detecting and characterizing mutations in large multi-exon genes such as the dystrophin gene. Mutations in the dystrophin gene result in both Duchenne and Becker muscular dystrophy (DMD and BMD), as well as X-linked dilated cardiomyopathy. Mutational analysis is complicated by the large size of the gene, which consists of 79 exons and 8 promoters spread over 2.2 million base pairs of genomic DNA. Deletions of one or more exons account for 55-65% of cases of DMD and BMD. A multiplex PCR method is currently the most widely available method for mutational analysis and it detects approximately 98% of deletions. However, detection of point mutations and small subexonic rearrangements has remained challenging. The disclosed method overcomes the problems associated with prior art DNA screening methods by allowing direct sequence analysis of a multi-exon gene in a rapid, accurate, and economical fashion.

The disclosed method provides for the identification and analysis of specific individual genomic mutations such as deletions, point mutations, frameshifts, or combinations thereof, in gene complexes with multiple exons/introns spanning large genomic regions.

As used herein, the term “deletion” refers to those genomic DNA sequences in which one or more nucleic acid bases has been deleted from the sequence and is no longer present in the gene.

As used herein, the term “point mutation” refers to a mutation resulting from a change in a single base pair in the DNA molecules, caused by the substitution of one nucleotide for another.

As used herein, the term “frameshift” refers to a loss or gain of some number of nucleotides which is not divisible by three (i.e., one or more codons).

The primary determinant of sequence specificity and base call quality is the uniform use of internal sequencing primers. The disclosed assay design is robust in that it can tolerate secondary, non-specific PCR amplification products, as opposed to assays that use a single set of primers or use secondary primers to universal sequences on the 5′ end of the PCR primers. An object of the method is the optimization a single 96 well plate assay in which all coding regions and promoters of the dystrophin gene are amplified in a single PCR plate. The PCR products are then purified in plate format using multi-channel pipetting robots, and two cycle sequencing plates prepared and processed. Sequencing can be routinely performed within 3 working days following DNA purification at a reasonable cost including both reagents and personnel costs. The one patient-one plate assay is designed for the requirements of both a rapid turnaround time for the assay, as well as making the assay scalable with a potential increase in demand.

Thus, an embodiment for the methods and compositions disclosed herein is a method designed to achieve PCR amplification and cycle sequencing of 96 distinct amplicons from a single individual using uniform thermal cycling parameters in a single vessel such as a 96 or 384 well thermal cycler microtiter plate. Alternatively, several individuals with multiple amplicons can be assayed in the same plate, e.g., four individuals with twenty-four distinct amplicons. The method comprises: designing PCR and sequence primers with software, performing a PCR reaction with the PCR primers on a DNA sample, performing a sequencing reaction with sequencing primers on the PCR products, electrophoretic separation and fluorescent detection of the sequencing reaction products on a capillary sequencer, and analyzing the DNA sequence with software.

In one aspect, disclosed herein is a method for characterizing the mutations in a multi-exon gene comprising: providing a sample of a patient's purified genomic DNA, plating the DNA in a 96 well plate followed by PCR amplification of gene-specific DNA fragments with a different PCR amplification primer set for each of the 96 wells under uniform amplification conditions. This is followed by cycle sequencing of the amplified DNA fragments with a different internal sequencing primer set for each well in a 96 well plate under uniform sequencing conditions. Samples from each sequencing reaction are then loaded onto an automated DNA capillary sequencer. Sequence data are then collected and analyzed with a computer using a mutation detection software program. A database is generated from the mutation sequence information, and with the software, the product sequence can be compared to other known sequences.

C. Genes:

The disclosed methods can involve the use of any genomic DNA sequence or any other nucleic acid sequence of interest. For example, a genomic DNA sequence to be detected herein can be derived from an organism, preferably a human patient and more preferably a human patient having or suspected of having a dystrophinopathy. The source of the genomic DNA from the organism to be tested can be from any tissue, such as peripheral lymphocytes.

The disclosed method is applicable to known or unknown genes, and should allow the development of widely-available assays for any number of large, multi-exon genes. Examples of some multi-exon genes which are candidates for the use of the disclosed method are NF-1, ATM, dysferlin, calpain, αβγδε sarcoglycans, collagens 6A1-3, Nebulin, and Titin. More preferred are those polymorphic genes associated with orphan diseases including but not limited to the dystrophin gene in DMD or BMD, the SOD-1 gene in Amyotrophic Lateral Sclerosis, NF-1 in von Recklinghausen neurofibromatosis, and dysferlin in limb-girdle muscular dystrophy type 2B.

D. Amplicons:

For the purposes of the disclosed methods, distinct regions of the nucleic acid sequence of interest, such as a sample of genomic DNA, can be identified for amplification. These regions of the nucleic acid of interest can each be amplified with a set of amplification primers. As such, these distinct regions of a nucleic acid sequence of interest can be termed amplicons. Also, as used herein, the term amplicon refers to the product of an amplification reaction upon a distinct region of a nucleic acid region of interest. Amplicons from a given nucleic acid sequence of interests or genomic DNA can be non-overlapping regions of the nucleic acid sequence of interest. Alternatively, amplicons can have overlapping portions in the nucleic acid sequence of interest. Also, an amplicon can be, for example, a single exon, a single exonic region or a proximal promoter sequence.

An amplicon can be of any length. For example, a amplicon can have an average length of, 0.5 kilobases (kb), 0.6 kb, 0.7 kb, 0.8 kb, 0.9 kb, 1.0 kb, 1.1 kb, 1.2 kb, 1.3 kb, 1.4 kb, 1.5 kb, 1.6 kb, 1.7 kb, 1.8 kb, 1.9 kb, 2.0 kb, 2.2 kb, 2.5 kb, 3 kb, 3.5 kb, 4 kb, 4.5 kb, 5 kb, 5.5 kb, 6 kb, 7 kb, 8 kb, 9 kb, 10 kb, 11 kb, 12 kb, 13 kb, 14 kb, 15 kb, 16 kb, 18 kb, 20 kb, 22 kb, 24 kb, 26 kb, 28 kb, 30 kb, 2 kb or more, 2.5 kb or more, 3 kb or more, 3.5 kb or more, 4 kb or more, 4.5 kb or more, 5 kb or more, 5.5 kb or more, 6 kb or more, 7 kb or more, 8 kb or more, 9 kb or more, 10 kb or more, 11 kb or more, 12 kb or more, 13 kb or more, 14 kb or more, 15 kb or more, 16 kb or more, 18 kb or more, 20 kb or more, 22 kb or more, 24 kb or more, 26 kb or more, 28 kb or more, 30 kb or more, about 2 kb, about 2.5 kb, about 3 kb, about 3.5 kb, about 4 kb, about 4.5 kb, about 5 kb, about 5.5 kb, about 6 kb, about 7 kb, about 8 kb, about 9 kb, about 10 kb, about 11 kb, about 12 kb, about 13 kb, about 14 kb, about 15 kb, about 16 kb, about 18 kb, about 20 kb, about 22 kb, about 24 kb, about 26 kb, about 28 kb, about 30 kb, about 2 kb or more, about 2.5 kb or more, about 3 kb or more, about 3.5 kb or more, about 4 kb or more, about 4.5 kb or more, about 5 kb or more, about 5.5 kb or more, about 6 kb or more, about 7 kb or more, about 8 kb or more, about 9 kb or more, about 10 kb or more, about 11 kb or more, about 12 kb or more, about 13 kb or more, about 14 kb or more, about 15 kb or more, about 16 kb or more, about 18 kb or more, about 20 kb or more, about 22 kb or more, about 24 kb or more, about 26 kb or more, about 28 kb or more, or about 30 kb or more. In some aspects, the amplicon has an average length of from about 1.0 kb to about 2.0 kb, from about 1.0 kb to about 1.8 kb, from about 1.0 kb to about 1.6 kb, from about 1.0 kb to about 1.4 kb, from about 1.0 kb to about 1.2 kb, from about 1.2 kb, to about 2.0 kb, from about 1.2 kb to about 1.8 kb, from about 1.2 kb to about 1.6 kb, from about 1.2 kb to about 1.4 kb, from about 1.4 kb to about 2.0 kb, from about 1.8 kb, from about 1.4 kb to about 1.6 kb, from about 1.6 kb to about 2.0 kb, from about 1.6 kb to about 1.8 kb, or from about 1.8 kb to about 2.0 kb. In another aspect, the amplicon can have an average length of from about 1.2 to about 1.4 kb.

While amplicons can be of any length (as measured by the number of nucleotides in the amplicon), it is useful to note that having larger amplicons will require fewer reaction chambers when practicing the methods disclosed herein. Conversely, the smaller the amplicon size, the more reaction chambers that are needed. For example, partitioning a nucleic acid sequence of interest into, say, 50 amplicons, will require more reaction chambers than it would if the nucleic acid sequence were partitioned into, say, 25 amplicons.

Also, there is no specific requirement that a certain number of amplicons be used in the methods disclosed herein. The number of amplicons will largely depend on the size of the nucleic acid sequence of interest or genomic DNA. In general, a large nucleic acid sequences of interest will typically result in a larger number of amplicons. Similarly, smaller nucleic acid sequences will typically result in less amplicons being used. However, in the disclosed methods, any number of amplicons can be used. In one aspect, the number of amplicons that can be used in the methods disclosed herein are about 48, about 96, or about 348. In another aspect, the number of amplicons that can be used are, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, or 348 amplicons. It is also, possible to perform the disclosed method on more than 348 amplicons, such as about 350, 400, 450, 500, 600, 750, 1000, 1250, 1500, 2000, 2500, 3000, 4000, or 5000 amplicons.

Also, according to the disclosed methods, a plurality of amplicons are amplified in a plurality of reaction chambers. It is useful for such amplification reactions to be conducted at similar or the same conditions. To this end, it can be beneficial to have amplicons of substantially similar lengths. In this way, the amplification conditions for each amplicon will be similar, and the amplification of more than one amplicon will be more efficient. For example, amplicons of similar lengths can be amplified to a similar extent at substantially the same temperature, with substantially the same amount of reagents, and with the same number of cycles.

E. Reaction Chambers:

The disclosed methods, either in whole or in part, can be performed in or on solid supports or in or on reaction chambers. For example, the disclosed amplification and sequencing steps (or any other operations of the disclosed methods) can be performed with the reaction mixture in or on solid supports or in or on reaction chambers. For example, the disclosed amplification and sequencing can be performed with the reaction mixture on solid supports having reaction chambers. A reaction chamber is any structure in which a separate reaction can be performed. Useful reaction chambers include tubes, test tubes, eppendorf tubes, vessels, micro vessels, plates, wells, wells of micro well plates, wells of microtitre plates, chambers, micro fluidics chambers, micro machined chambers, sealed chambers, holes, depressions, dimples, dishes, surfaces, membranes, microarrays, fibers, glass fibers, optical fibers, woven fibers, films, beads, bottles, chips, compact disks, shaped polymers, particles, microparticles or other structures that can support separate reactions. Reaction chambers can be made from any suitable material, such as solid support materials. Such materials include acrylamide, cellulose, nitrocellulose, glass, gold, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid supports preferably comprise arrays of reaction chambers. Solid supports and reaction chambers can be porous or non-porous. A useful form for reaction chambers is a microtiter dish. A particularly useful form of microtiter dish is the standard 96-well type. In some embodiments, a multiwell glass slide can be employed.

In connection with reaction chambers, a separate reaction refers to a reaction where substantially no cross contamination of reactants or products will occur between different reaction chambers. Substantially no cross contamination refers to a level of contamination of reactants or products below a level that would be detected in the particular reaction or assay involved. For example, if nucleic acid contamination from another reaction chamber would not be detected in a given reaction chamber in a given assay (even though it may be present), there is no substantial cross contamination of the nucleic acid. It is understood, therefore, that reaction chambers can comprise, for example, locations on a planar surface, such as spots, so long as the reactions performed at the locations remain separate and are not subject to mixing. Some useful forms of the disclosed methods can use reaction chambers that can be sealed to allow thermocycle reactions (for example, PCR and cycle sequencing) of small volumes.

Methods for immobilization of nucleic acid sequences to solid-state substrates are well established. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

In one aspect, the disclosed method can involve simultaneously performing various reactions, such as amplification and sequencing, on a plurality of amplicons. It is preferable that these reactions be conducted on an a plurality of amplicons where each amplicon has been allocated to a separate reaction chamber. That is, one amplicon can amplified and/or sequenced in one reaction chamber. However, although not preferred, more than one amplicon, i.e., 2, 3, 4, 5, 10, 20, etc., can be amplified and/or sequenced in one reaction chamber. Also, the same amplicon can be amplified and/or sequenced in multiple reaction chambers. This could be done, for example, when the additional reaction chambers are used as controls or duplicates. It is preferable that multiple reactions be conducted in or on a single solid support, preferably with a plurality of reaction chambers. That is, multiple amplicon, such as all of the amplicons for a multi-exon gene, can be amplified and/or sequenced on one solid support. However, multiple amplicons for a multi-exon gene can also be amplified and/or sequenced on multiple solid supports.

The disclosed methods can involve the use of multiple reaction chambers. For example, in one aspect, the disclosed methods can involve amplifications reactions that are simultaneously carried out on the contents of various reaction chambers. Similarly, the disclosed methods can involve sequencing reactions that are simultaneously carried out on the contents of various reaction chambers. The number of reaction chambers can be related to the number of amplicons, such as one reaction chamber for each amplicon. While the number of reaction chambers can be the same as the number of amplicons, additional reaction chambers can also be used for controls or duplicates. In one aspect, the disclosed methods can utilize 48, 96, or 348 reaction chambers. In another aspect, the disclosed methods contemplates that 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, or 348 reaction chambers are used. It is also, possible to perform the disclose method on more than 348 reaction chambers, such as about 350, 400, 450, 500, 600, 750, 1000, 1250, 1500, 2000, 2500, 3000, 4000, or 5000 reaction chambers.

In one aspect of the disclosed methods, a nucleic acid sample (such as a genomic sample) containing the nucleic acid sequence of interest (such as a multi-exon gene) is contacted with, i.e., placed in or immobilized on, a reaction chamber or solid support before any amplification primers are added. Alternatively, amplification primers can be contacted with the reaction chamber or solid support prior to the introduction of any nucleic acid samples. More generally, components present in the reactions disclosed herein can be mixed, added or combined in any order, in any combination, or simultaneously.

F. Amplification and Sequencing Primers:

Amplification and sequencing reactions can be performed on a plurality of amplicons in a plurality of reaction chambers. As such, these amplification and sequencing reactions utilize sets of amplification primers and sets of sequencing primers. The PCR amplification and sequencing primers are selected to be complementary to the different strands of each specific sequence to be amplified. Primer's can be designed using any known primer prediction software program such as Oligo, GeneFisher, Web Primer or Primer 3 software (a primer prediction program with user-definable parameters for Tm, GC-hairpins, etc.).

For primer prediction of a multi-exon gene, such as dystrophin, dysferlin, calpain, or collagen VI, the genomic sequence is first prepared by masking all known human sequence repeats using the RepeatMasker program. Sequence repeats are re-analyzed when choosing sequence primers and unique repeats are unmasked. The genomic sequence is also masked when choosing sequence primers by a Perl script to eliminate single base repeats (AAAA or GGGG) occurring in the sequence primer. Perl script uses the RNA cross-match output (pair-wise Smith-Waterman comparison) of the mRNA against the genomic sequence to isolate the exon sequence and flanking genomic sequence. Size parameters passed to the Perl script determine the size of the PCR product. The Perl script generates a Primer 3-formatted sequence file. Primer 3 can generate four potential primer sets, and the primers are cross-matched against the consensus genomic and primer positions relative to the exons. An example of the Perl script is shown in the Program Listing below.

According to the disclosed methods, a set of right and left amplification primers are used for each amplicon. It is preferable that a different set of amplification primers be used for each amplicon. The sequencing primers are preferably internal to the PCR primers, increasing the tolerance to non-specific amplification products in the PCR stage. Just a single sequencing primer can be used. Preferably, however, two sequencing primers are used. The two sequencing primers can be forward and reverse primers or, alternatively, two forward primers or two reverse primers. The use of a forward and reverse internal sequencing primer can relax the stringency needed to get robust amplification of multiple different amplicons under uniform thermal cycling conditions.

Primers for use in the disclosed methods are oligonucleotides having sequence complementary to the target sequence, such as a nucleic acid sequence of interest, an amplicon of a nucleic acid sequence of interest, or an exon or proximal promoter of a nucleic acid sequence of interest. This sequence is referred to as the complementary portion of the primer. The complementary portion of a primer can be any length that supports specific and stable hybridization between the primer and the target sequence under the reaction conditions. Generally, this can be 10 to 35 nucleotides long or 16 to 24 nucleotides long. In some aspects, the primers can be from 5 to 60 nucleotides long, and in particular, can be 5, 6, 7, 8, 9, 10, 11, 12, 13, 14, 15, 16, 17, 18, 19, and/or 20 nucleotides long.

The disclosed amplification and sequence primers can have one or more modified nucleotides. Such primers are referred to herein as modified primers. Modified primers have several advantages. First, some forms of modified primers, such as RNA/2′-O-methyl RNA chimeric primers, have a higher melting temperature (Tm) than DNA primers. This increases the stability of primer hybridization and will increase strand invasion by the primers. This will lead to more efficient priming. Also, since the primers are made of RNA, they will be exonuclease resistant. Such primers, if tagged with minor groove binders at their 5′ end, will also have better strand invasion of the template dsDNA.

Chimeric primers can also be used. Chimeric primers are primers having at least two types of nucleotides, such as both deoxyribonucleotides and ribonucleotides, ribonucleotides and modified nucleotides, or two different types of modified nucleotides. One form of chimeric primer is peptide nucleic acid/nucleic acid primers. For example, 5′-PNA-DNA-3′ or 5′-PNA-RNA-3′ primers may be used for more efficient strand invasion and polymerization invasion. The DNA and RNA portions of such primers can have random or degenerate sequences. Other forms of chimeric primers are, for example, 5′-(2′-O-Methyl) RNA-RNA-3′ or 5′-(2′-O-Methyl) RNA-DNA-3′.

Many modified nucleotides (nucleotide analogs) are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and 0-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Primers composed, either in whole or in part, of nucleotides with universal bases are useful for reducing or eliminating amplification bias against repeated sequences in a target sample. This would be useful, for example, where a loss of sequence complexity in the amplified products is undesirable. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C 10, alkyl or C2 to C10 alkenyl and alkynyl. 2′ sugar modifications also include but are not limited to —O[(CH₂)n O]m CH₃, —O(CH₂)nOCH₃, —O(CH₂)nNH₂, —O(CH₂)nCH₃, —O(CH₂)n —ONH₂, and —O(CH₂)nON[(CH₂)nCH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Primers can be comprised of nucleotides and can be made up of different types of nucleotides or the same type of nucleotides. For example, one or more of the nucleotides in a primer can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. The nucleotides can be comprised of bases (that is, the base portion of the nucleotide) and can (and normally will) comprise different types of bases. For example, one or more of the bases can be universal bases, such as 3-nitropyrrole or 5-nitroindole; about 10% to about 50% of the bases can be universal bases; about 50% or more of the bases can be universal bases; or all of the bases can be universal bases.

A particularly useful embodiment of the disclosed methods is a method for detecting mutations in the dystrophin gene. The disclosed method is at least as sensitive as DOVAM screening, and has been successful in identifing at least one mutation undetected by the DOVAM method. Sequencing specificity is gained by uniform use of a second, internal set of sequencing primers. Sufficient sequencing specificity is obtained without optimization of individual amplicon conditions. The disclosed method results in complete double-stranded sequencing coverage of all known coding regions and 7 of the 8 tissue-specific promoters. Although the dystrophin muscle isoform coding region consists of 11.1 kb, the disclosed sequencing method analyzes an average of nearly 110 kb of sequence, allowing detection of polymorphisms in flanking intronic regions as well as the 3′ UTR and 5′ regions. The disclosed method allows detection of the approximately 2% of patients with exonic deletions not detected by the widely available multiplex PCR technique. The disclosed method gives highly reproducible and accurate results, and can be performed economically on single samples as described in further detail hereinafter.

The amplification and/or sequence primers can be any size that supports the desired enzymatic manipulation of the primer, such as amplification and/or sequencing. A typical primer would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides long.

G. PCR:

Various thermocycling parameters and PCR enzyme/buffer combinations that are known in the art may be used to arrive at a single condition for amplification of DNA fragments (Maniatis, T., E. F. Fritsch and J. Sambrook. 1982. Molecular Cloning: A Laboratory Manual). After the PCR reaction is complete, the amplification products from each reaction chamber can optionally be purified. Purification techniques are known in the art. The examples below illustrate techniques for such purification. The purified or unpurified amplification products from each reaction chamber can be transferred to a second reaction chamber. Alternatively, the purified or unpurified amplification products can be left in the same reaction chamber.

H. Sequencing:

According to the disclosed methods, the amplicons can be sequenced under uniform temperature and conditions. The internal sequencing primers are added to a reaction chamber. This reaction chamber may be the same reaction chamber used in the PCR amplification, and will thus contain the purified or unpurified amplified amplicons. Alternatively, the internal sequencing primers can be added to a second reaction chamber prior to, during, or after amplified amplicons have been transferred from the original reaction chamber used in the amplification reaction.

The disclosed method is adaptable for any sequencing method or detection method that relies upon or includes chain extension. These methods include, but are not limited to, sequencing methods based upon Sanger sequencing, and detection methods, such as primer oligo base extension (PROBE) (see, e.g., U.S. Pat. No. 6,043,031 and U.S. Pat. No. 6,235,478), that include a step of chain extension. Automated techniques have also been developed to increase the throughput and decrease the cost of nucleic acid sequencing methods, e.g., U.S. Pat. No. 5,171,534; Connell et al., Biotechniques, 5(4): 342-348 (1987); and Trainor, Anal. Chem., 62: 418-426 (1990). Numerous useful sequencing techniques, including, for example, cycle sequencing, are known and can be adapted for use in the disclosed method.

I. Kits:

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for the detection and, optionally, characterization, of mutations in multi-exon genes, the kit comprising sets of amplification primers and sets of internal sequencing primers that are designed for the particular multi-exon gene. The kits also can contain reaction chambers or solid supports, amplicons from the multi-exon gene, amplification and/or sequencing reagents, solvents, probes, markers, detection tags, and the like. Also disclosed are kits for the detection and, optionally, characterization, of mutations in the dystrophin gene, the kit comprising sets of amplification primers and sets of internal sequence primers. The kits can also contain amplicons from the dystrophin gene, reaction chambers or solid supports, reagents, solvents, probes, markers, detection tags, and the like.

It is also contemplated that each step of the disclosed methods can be in a separate kits. For example, there can be one kit for the amplification of amplicons of a nucleic acid sequence of interest and another kit for the sequencing of such amplicons.

J. Mixtures:

Disclosed are mixtures formed by performing or preparing to perform the disclosed method. For example, disclosed are mixtures comprising an amplicon from a nucleic acid sequences of interest and a set of amplification primers. Also, disclosed are mixtures comprising an amplicon and a set of sequence primers.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

K. Systems:

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising automated delivery systems, such as robots, that deliver compositions, such as amplification primer sets, sequencing primer sets, reagents, solvents, and the like, to each of a plurality of reaction chambers or solid supports. Also, disclosed are reaction chambers or solid supports that contain or are associated with amplicons from a nucleic acid sequence of interest, i.e., a multi-exon gene. Also, disclosed are reaction chambers or solid supports that contain or are associated with amplification primer sets or sequence primer sets.

L. Data Structures and Computer Control

Disclosed are data structures used in, generated by, or generated from, the disclosed method. Data structures generally are any form of data, information, and/or objects collected, organized, stored, and/or embodied in a composition or medium. A nucleic acid library stored in electronic form, such as in RAM or on a storage disk, is a type of data structure.

The disclosed method, or any part thereof or preparation therefore, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

The objects of the invention have been achieved by a series of experiments some of which are described by way of the following non-limiting examples.

### Specific Embodiments

Disclosed is a method for characterizing a genomic DNA fragment by Single Condition Amplification/Internal Primer (SCAIP) sequencing comprising the steps of:
providing a PCR reaction plate wherein the wells of each plate contain the genomic DNA fragment;
adding to each of the wells a different set of left and right PCR primers complementary to a nucleotide sequence within the genomic DNA fragment and performing a PCR reaction at a uniform temperature;
purifying PCR fragments from each of the wells, adding the fragments to a corresponding well of a cycle sequencing reaction plate to which is added left and/or right internal sequencing primers corresponding to the PCR fragments, and sequencing at a uniform temperature;
purification of sequencing products followed by electrophoretic separation and fluorescent detection of nucleotides on a sequence analyzer; and
nucleotide sequence characterization.

Also disclosed is a method for identifying a mutation in a multi-exon gene by Single Condition Amplification/Internal Primer (SCAIP) sequencing comprising the steps of:
providing a sample of a patient's purified genomic DNA comprising the multi-exon gene,
plating the DNA in a 96 well plate followed by PCR amplification of gene-specific DNA fragments with a different PCR amplification primer set for each of the 96 wells under uniform amplification conditions, wherein each primer set is complementary to a single exonic region or a proximal promoter region of the gene,
cycle sequencing of the amplified DNA fragments with a different internal sequencing primer set for each well in a 96 well plate under uniform sequencing conditions,
electrophoretic separation of sequencing reaction products and fluorescent detection of nucleotides on a sequence analyzer; and
analyzing the nucleotides for mutations and comparing to other known nucleotide sequences.

Also disclosed is a method for diagnosing a distrophinopathy in a patient by Single Condition Amplification/Internal Primer (SCAIP) sequencing comprising the steps of:
providing a sample of the patient's purified genomic DNA comprising the dystrophin gene,
plating the DNA in a 96 well plate followed by PCR amplification of gene-specific DNA fragments with a different PCR amplification primer set for each of the 96 wells under uniform amplification conditions, wherein each primer set is complementary to a single exonic region or a proximal promoter region of the gene,
cycle sequencing of the amplified DNA fragments with a different internal sequencing primer set for each well in a 96 well plate under uniform sequencing conditions,
electrophoretic separation of sequencing reaction products and fluorescent detection of nucleotides on a sequence analyzer; and
analyzing the nucleotides for mutations and comparing to other known nucleotide sequences for the gene.

Also disclosed is a method for identifying a mutation in a multi-exon gene by Single Condition Amplification/Internal Primer (SCAIP) sequencing comprising the steps of:
providing a sample of a patient's purified genomic DNA comprising the multi-exon gene,
plating the DNA in a 96 well plate followed by PCR amplification of gene-specific DNA fragments with a different PCR amplification primer set for each of the 96 wells under uniform amplification conditions, wherein each primer set is complementary to a single exon or a proximal promoter region of the gene, cycle sequencing of the amplified DNA fragments with a different internal sequencing primer set for each well in a 96 well plate under uniform sequencing conditions,
electrophoretic separation of sequencing reaction products and fluorescent detection of nucleotides on a sequence analyzer; and
analyzing the nucleotides for mutations and comparing to other known nucleotide sequences.

Also disclosed is a method for diagnosing a distrophinopathy in a patient by Single Condition Amplification/Internal Primer (SCAIP) sequencing comprising the steps of:
providing a sample of the patient's purified genomic DNA comprising the dystrophin gene,
plating the DNA in a 96 well plate followed by PCR amplification of gene-specific DNA fragments with a different PCR amplification primer set for each of the 96 wells under uniform amplification conditions, wherein each primer set is complementary to a single exon or a proximal promoter region of the gene, cycle sequencing of the amplified DNA fragments with a different internal sequencing primer set for each well in a 96 well plate under uniform sequencing conditions,
electrophoretic separation of sequencing reaction products and fluorescent detection of nucleotides on a sequence analyzer; and
analyzing the nucleotides for mutations and comparing to other known nucleotide sequences for the gene.

The multi-exon gene can be dystrophin, SOD-1 NF-1, ATM, dysferlin, calpain, αβγδε sarcoglycans, collagen 6A1-3, Nebulin, and Titin. The PCR primers can be selected from the group of primer sets as shown in Table 1. The sequencing primers can be selected from the group of primer sets as shown in Table 2. The dystrophinopathy can be Duchenne Muscular Dystrophy (DMD) and Becker Muscular Dystrophy (BMD). The mutation can be a deletion, point mutation, frameshift, duplication or combinations thereof.

Also disclosed is a PCR primer set which recognizes a single exon or a proximal promoter for the dystrophin gene as shown in Table 1. Also disclosed is a sequencing primer set which recognizes a single exon or a proximal promoter for the dystrophin gene as shown in Table 2.

Also disclosed is a PCR primer set which recognizes a single exon or a proximal promoter for the CAPN3 and DYSF genes as shown in Table 6. Also disclosed is a sequencing primer set which recognizes a single exon or a proximal promoter for the CAPN3 and DYSF genes as shown in Table 7.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### A. Example 1

### Single Condition Amplification/Internal Primer (SCAIP) Sequencing Method

The genomic organization of the dystrophin gene was assembled from contigs downloaded from the UCSC Human Genome Browser (Kent et al. (2002) Genome Res 12:996-1006) (see also the International Human Genome Sequencing Consortium 2001 (Lander et al. (2001) Nature 409:860-921)). Assembly and exon-intron annotation was performed using task-specific Perl scripts. The completed assembly reveals that the DMD region is currently contiguous and gap-free for the dystrophin Dp427m muscle isoform (NM-004006) spanning 2.09 Mb, and the dystrophin Dp427c brain isoform (NM-000109) spanning 2.22 Mb of chromosome Xp21.2. Primer systems for polymerase chain reaction (PCR) were designed to amplify DNA fragments which span each exon and 7 of the 8 promoters (Dp427m, Dp427p, Dp427c, Dp4271, Dp260, Dp140, Dp116) (Table 1). Each amplicon was designed for an optimal size range of 1.2 to 1.4 kb with the exon, including unique promoters, centered within the amplicon, with the exception of exon 79 which was broken into 7 fragments to maintain uniform conditions. These were designed to produce 93 amplicons with a nearly universal size; this uniformity allows one to predict likely amplification conditions using a single set of PCR temperatures.

TABLE 1

Primer Pairs Used to Amplify the DMD Exons and
Promoters and Sizes of PCR Products.

| | | | |
|---|---|---|---|
| | | | Product |
| | | | Length |
| Exon | Forward | Reverse | (bp) |

| | | | |
|---|---|---|---|
| M1 | AATTGGCACCAGAGAAATGG | TCATGTGTTTAGTTCTATCGCAAA | 1223 |
| 2 | TCATTTCTCCATGGTTGGGT | TGACATCCCAATAAACCTCCA | 1400 |
| 3 | GCTCTCACAGGGTTGTTTCA | GAAGGGCAAAGATAAGAGACGA | 1347 |
| 4 | GGGAACCAAAGTGATTGAGG | TGGTTGGAGACAGCGTTTAAT | 1367 |
| 5 | CAGGAGACACAGAGATTTGCC | TCGGAAGACCCTATGCTCAG | 1148 |
| 6 | GCTTGCGTTAAATGATGGTATG | TTGATTTGCTGTTCCAGTGC | 1391 |
| 7 | GCGTAGATTATTTGTCATCTTCAGG | TGAGTAACCATCCAACAGAGGA | 1245 |
| 8 | TTATCCCATGCACCACAATG | CAAGCCAATGTCATGGAAGA | 1360 |
| 9 | CTGCTGAATGTGTGGAGAGC | ACATTTCATTCCCACGCTGT | 1298 |
| 10 | GGCCTTCTGGAAATAAAGGC | AAACTTGTGGCCCATTTAGA | 1349 |
| 11 | GCCAACAGGAATACGAAAGC | TTCAAATCCACAGTTGGCAC | 1348 |
| 12 | TGCAGAATCACTCCTATATGGTC | CATACCCTGCGTTGTTTCTCA | 1336 |
| 13 | GGAGAACATCCTGCTGTACCTT | TAGCAAGGGCTTTCTCTCCA | 1184 |
| 14 | TTCCTTTGCATAGAAAGCATCA | AACCGTCGCTTGTAACTCTCA | 1398 |
| 15 | CCAAATGGTAGGCAATTCTCA | AATGTCAGGATAACCGTCGC | 1148 |
| 16 | CAGCATTTCAGAATGGCAAG | TGAAATCAGCAGTCTATGGCA | 1177 |
| 17 | TGTCTCCAGTGATGAATATGGG | TGCGCAGACTGAGACATCAT | 1333 |
| 18 | AAGCTCTGACATGCAAGCAC | ACTGAGAAAGGCTGGACACC | 1171 |
| 19 | TTGTCTTCCTTGGAAATAGGAG | TTTGGAAATAGCATTATCCCTGA | 1399 |
| 20 | ATTTAAACTAATTTCCAAGCCCA | ACACTATCCGGTGTGGTTCC | 1232 |
| 21 | GCCTGTTTGGTCAGGACAAG | GCTGAGTTTCAGTTGCCACA | 1247 |
| 22 | TTGCAATTGGGATTAACAATG | CCCACCAGTTTGAGAATGTG | 1117 |
| 23 | ATCCTTGAATCCCACCATAAT | CAGCAGAAATGAAAGGTAATATAGGA | 1168 |
| 24 | GGGAAAGAATCATGGGTGAG | CTTCCTGCTGCATGACAATG | 1256 |
| 25 | CATTGTCATGCAGCAGGAAG | ATGTGTCGAAGAGGCCAAAC | 1081 |
| 26 | TGAATTATCATCATCGGGCA | CCTTGTCACAATCCTTGAACC | 1271 |
| 27 | CACAAATCCATACCTCCATGC | TTGAGGCACCTGCTTTCTTT | 1078 |
| 28 | TCCATATTCACGATGATGTTTACC | GAGCTTGAATGATTAAATGTCAGAA | 1338 |
| 29 | GCGAGTAGGCAGTCTCTGCT | TCTTGCACATTCTAGGAAATCAG | 1380 |
| 30 | GATCATGCAAAGCTGGTTGA | TGCTTTCCAACAATGCCATA | 1347 |
| 31 | AGTATCTGCCGGAAGCCAT | GCAAGTGCATCTTCACTTCATC | 1398 |
| 32 | CATGGTAGAGGTGGTTGAGGA | ATTCGGTGTTGTCTTGAGGC | 1330 |
| 33 | TTCATCCAAATTTATGGCTAGAAT | AGTTGAGCGAAGTGAGATGGA | 1203 |
| 34 | CTGAGAACAGGAGCACAGGA | GCTGTGTCATTTGGTGATGG | 1324 |
| 35 | GGGCAGTTTCTTATTTGTGGA | TACCACCATTGACAAAGGCA | 1268 |
| 36 | CCATACAGAAAGCCGTTTCA | GACAGGGCATCCTAACAGTCA | 1242 |
| 37 | ACTTCAACCTCTGTGACCCG | ACCCTAGACCGTGCAGAAGA | 1244 |
| 38 | TGCATCACCAACCAAACTGT | CAGAGGTGATGGCAGTGAAA | 1399 |
| 39 | GGTTTCAGAAATGAAGCAGGA | TCCTGCACAAACCAGATGAG | 1290 |
| 40 | AGCCTTGGAAGGAGAAGCAT | ATTCCTCTGGTGTCTTGGGA | 1398 |
| 41 | AGCCCATTCATTTCATCAGAG | ATGGCTTATGCAGGTTGACA | 1155 |
| 42 | GAAATTTAAATGCCGGTTGC | GCTTCCAGGAAACCATTTGA | 1371 |
| 43 | CACCATTTGCTACCTTTGGG | TTCAGCTCATTTGTCTGAATTG | 455 |
| 44 | GGATTAAAGAAGGCATCGCA | GGTTCCAACATAAAGCCGAA | 1372 |
| 45 | ATCTTGATGGGATGCTCCTG | CATTTGGCTTTCTGTGCCTT | 1370 |
| 46 | CAGATATAATGACATAATGTTGTTAGA | GCAATCCAGATCTTCCCTAAG | 1264 |
| 47 | GTCTTGGGAAAGGGCATACA | ATAGTATGCAAGGTGGAAAGATG | 1374 |
| 48 | CCTATAATCATTCTGTTACAGTCTAC | GAAGCCTGTCAGTTTACAAGAAC | 1370 |
| 49 | TGCTTTAAGTGTTTACCCTTTGG | CTGACCTGGCTTTCCATCTC | 1247 |
| 50 | GCTAGTTGCTGAGAGGGAACTG | AAGCCAGCATTAACATTGCC | 1244 |
| 51 | TTCATTGGCTTTGATTTCCC | GAAGGCAAATTGGCACAGAC | 1198 |
| 52 | GATGCTCTCCAAACTTGCCT | AAGTTCCTGCCCACCCTACT | 1298 |
| 53 | CAGAAACTAATATTTGCCATCAAAA | GAGAAGAATGAGCTGGGCTG | 1162 |
| 54 | AAGCCTCCTCTCTGCACTTG | CGAGTCATATTGCCCTCCAC | 1378 |
| 55 | AGCAGCATCAAAGACAAGCA | CGACAAATTCAGCCATCTCA | 1159 |
| 56 | GGCCAAGTGCAATCTTGTTT | TTCCTCCACGGAACTATTGC | 1380 |
| 57 | GGCTGCCTAGGGTGTAGAAA | TTGATTGCATGTTGAAATGAC | 1375 |
| 58 | TCCGCAATTCCTACATCCAT | GCTTTCGTAGAAGCCGAGTG | 1399 |
| 59 | ACCAGGAGCCCAGAGGTAAT | AGGGCAACACATTAACAGCC | 1357 |
| 60 | CCATTGTTATAATACTACCACAAGAG | GTGGCAATTCACATCTTCCA | 1309 |
| 61 | CCAAATTTAAGCCTTGCCTG | TGAACTGAACTGATAGGCAGAAA | 1325 |
| 62 | GCAAAGATCATTCATTTGACCA | AGTCGAGGACTGCTGCTTTC | 1250 |
| 63 | GCTTCATTCAGGCCCAAGTA | GACAAACCAGACATCTGGACA | 1369 |
| 64 | AGTTTATGGGCTTGTGGATGA | GCACACAGACCCTCAGACAA | 1176 |
| 65 | GCAGAGAGATGCTGAGGTGA | ATCTCCCTTGTGTGCAATCC | 1350 |
| 66 | TGTGTTATGTGGCCTGAAGTAA | CAACTGCAGCCTTTCACAAT | 1275 |
| 67 | CCTGTGGGAACACATACATGA | GCAATGGGACAGGATAGGAA | 1192 |
| 68 | CAGACAGAATCAACAGGGCA | TGGTGCAAAGTGAATGAGAGA | 1242 |
| 69 | TTTGAAGATGAATCGTATCAGTCAA | CTACATCCTTGCCATTTCCC | 1335 |
| 70 | TCCTCCCAGATATTTGCCTG | GGAAAGCAATAGCCAAACCA | 1222 |
| 71 | CTCTCAGCTGAACACCCTCC | CCTGATAAACAGTCCGCACA | 1210 |
| 72 | CTTGCTGCTGAATTGGAAGA | TTGACAAAGGATGGATGGAC | 1318 |
| 73 | ACTTGCCCTCTAACGTGCAT | GGCAGGTTTGGTCAAAGATT | 1383 |
| 74 | CTTGGTGGCCAAAGCATTAT | GGGCTCAACCAAAGAGATGA | 1354 |
| 75 | TGGCATTATCTCCTTGAGGG | TCCCAGAAAGCCAGAACTATG | 1318 |
| 76 | CATAGTTCTTTAAGCCTCCATCG | CCAACCAAATCCTCTCCCTT | 1301 |
| 77 | TGAGGAGACAGCACTGCAAG | AAAGGGCACCTCATAATTAACTCTT | 1397 |
| 78 | CTCTGTGGCTTGCCCATTAC | TGAAGGGTACGTTGAGATGATG | 1389 |
| 79a | GAAAATAGCCACCTCCACCA | ATATCACGCCAAAAGGATGC | 1153 |
| 79b | TCACTCATAGCCAAGGTGGA | AAGCAGGTAAGCCTGGATGA | 1227 |
| 79c | TTACAACTCCTGATTCCCGC | TCACAAATGTGATGGGGCTA | 1380 |
| 79d | ATATGGAACGCATTTTGGGT | CCTGTGTGGAACTACTCGCA | 1206 |
| 79e | GCCAGGAGGAAACTACACCA | GGTCCAGCGTCACATAAAGG | 1295 |
| 79f | ACTCCCAAGCAGTAGCAGGA | CATGCCATGTGATGTTTATGC | 1319 |
| 79g | AGCCCATGAACTGTGTTTCC | AGCAATGAGGATGATTGATTGA | 1376 |
| Mp1 | GGGCACTTATACTCTGGGCA | CGCCTTCTCTCAAGTTGG | 1351 |
| Mp2 | TCAACTAAGGCTGAATGGCA | ATGCCCAGAATAATCCATGC | 1370 |
| Lp1 | CCATATCTAGAAGCTTTATTCTGTTTT | GAATCTGCTTTACAGTGGTTGAG | 708 |
| Pp1 | TGATCAGATGGGGATTGACA | TTCATTAAAGCCACAACCCA | 1324 |
| Cp1 | GCATACAGGGTGCCAGACTT | TAGACCAGCTGGGTCGACAT | 1399 |
| 260p1 | CTCAGTCATGCTCTGTGGGA | ATCAAAACAACCCCATGGAA | 1183 |
| 140p1 | CAATAGCCCCATTGCTCAGT | AAGAGGGCACAAGCTTTGAA | 1263 |
| 116p1 | CGTTCTGCAAGAATCCCAAT | TCTGACCATAAAAGCGTGGA | 1322 |

The primer sequences in Table I are SEQ ID NOs: 1-186, respectively (forward primer, reverse primer, from top to bottom).

Fifteen picomoles of each primer was aliquoted into individual wells of a 96-well tray, evaporated to dryness in a speed vac system, and stored in a −20 E C freezer until use. For PCR amplification, 10 μg of patient template DNA was aliquoted into a master PCR mixture and subsequently 25 μl of the mixture was aliquoted into the 96 well dish with dry primers. The PCR was carried out in a thermocycler for 25 cycles under the following conditions: denaturation at 94° for 20 s, annealing at 55° for 30s, and extension for 68° for 4 min, followed by a final extension at 68° for 7 minutes.

To validate PCR amplification and to detect any deletions, 3 μl of the PCR product was run on a 0.75% agarose/Ethidium Bromide gel. The resulting gel was photographed and analyzed for absence of one or more bands. Because the absence of a single band may result from a primer site polymorphism, in such cases PCR was repeated using (1) the same primers, (2) internal sequencing primers, and (3) combinations of original and internal primers. The absence of more than one adjacent exon is interpreted as being consistent with a multiexon deletion. The PCR products were then transferred and bound to a 96-well filter plate (Millipore MAFB 1.0:M glass fiber type B filter) in the presence of a 5 M guanidine HCl/potassium acetate solution. Wells were washed four times with 80% ethanol to remove unincorporated primers, nucleotides, and excess salt, followed by elution of the fragments with warm nanopure H₂O.

Internal sequencing primers were designed to anneal to unique intronic flanking sequences, with attention to specific 3′ sequence for each primer (Table 2). As with the PCR reaction, the primers were stored in 384 well plates so that both PCR set-up and sequence reaction set-up could be performed with multi-channel pipettors and pipetting robots.

TABLE 2
Internal Primers Used to Sequence the DMD Exons and Promoters.

| | | | Primer |
|---|---|---|---|
| | | | Distance |
| Exon | Internal Primer A | Internal Primer B | (bp) |
| M1 | CACTGTGCTATTCTGGTTTGGA | TTTATGCTTCTTTGCAAACTAGTG | 595 |
| 2 | ATTTTAATTTGGATGCCCCA | TCTTCTTCTGCTGGGTGACA | 563 |
| 3 | TTACTCTTGCTATCAAACTAATTCAA | TTTTCTGCAGGCGGTAGAGT | 501 |
| 4 | GCTAAAAACGTACCAGGCCA | GGAGCAGCCTATCAGGTCAG | 503 |
| 5 | TCCAGTTGACCTCTTTAATCTGC | CCGTGATGATCCTTAACATTTC | 516 |
| 6 | TGGCATAGATACCAATGAATCAG | TGTATCCCATAGAACACTGGAAAA | 562 |
| 7 | AGGACTATGGGCATTGGTTG | TTTTCCTAAAAGTCTTCACTGCAA | 461 |
| 8 | TGCTCATCTCATTGGTCTGC | CAATGAAGCAAAATTGAAAAGG | 560 |
| 9 | AAGTGCCTTCATTCTGGGAG | GAAACCATTACGGGAATTCAT | 542 |
| 10 | GGATTTTGACCGCTATTTGAA | GTTGGCCGATCAGGTAGAAA | 595 |
| 11 | GTGGTTTTGGGATTCTGCAA | CAGTGCATCTATCTAACATCTGCTC | 548 |
| 12 | AATAGTTCCGGGGTGACTGA | GGAGGGGACTTATTCAAGCC | 509 |
| 13 | TGGCTTGGAATGGTTTTAGG | GATTTTACCCATCCGCAGTT | 475 |
| 14 | TTGCTTGTCTCTTTGCTTTTC | CATACGGCCAGTTTTTGAAGA | 547 |
| 15 | TCGATGGGCAAACATCTGTA | TTGAAAAACAAAGTTGAAAATCCA | 505 |
| 16 | GAACTTTTGATCCTTTGCGG | TCACCACCATTCTCCAACAA | 493 |
| 17 | TGTTGAGATTACTTTCCCTTGC | TTGCGATAGTGATTTCTTGTGA | 571 |
| 18 | AACAGGGAAAATAGTGCTGCT | GGCATCCCTAGTCAGTCACAG | 491 |
| 19 | TCATGAAAATGGCTCATGCT | CCACATCCCATTTTCTTCCA | 497 |
| 20 | TTGTTGTGACGCAAGTCTGA | TTGCGCTTAGCTAAATCCTT | 565 |
| 21 | GGCTGGTGATAGAGGCTTGT | TCACAAAATTATTATGAGGACAAAAA | 544 |
| 22 | ATGTGTAAGGTCCCTGGCAT | TTTTCATTTGCTCAATGGG | 475 |
| 23 | TCAGAAAAATACATATGGAGTGTTAAA | AAGGAATAAGCAAATCGCCA | 612 |
| 24 | GCCTCAAGAACTACTTAGAGACATCC | AGGCAATGTTTTGTCAGTTCC | 581 |
| 25 | CCCACTGGATTCATGCCATA | TTTTAGGATCAAAATAAGATGAATGTG | 583 |
| 26 | TGAGTGTATCTGATCCCCATGA | TCTGATCCCCATGAGTTATTTTC | |
| 27 | TTTATGGAAGAGACTGGAGTTCA | GGAGAAAATTTATAGGATTTTATGACC | 672 |
| 28 | TTTCTTAATGACTTTTGATTGTAGAGG | GAAGCCATTTAAACCCTTTGC | 534 |
| 29 | GCAAAAATGCTCCTTGGTGT | CAGTGTCTGGCATTGGATTG | 446 |
| 30 | GGAGGAACATTCGACGTGAG | TCCTACCTACCTCCAAATAGTCAAA | 638 |
| 31 | CCCATAGGGAAGAAATAAATCG | CATACATTTGGGAGAATGATTCAG | 618 |
| 32 | TCCTGTGTTGGATGAATGGA | GCCACAATACATGTGCCAAT | 483 |
| 33 | ACCGCTGCAAAATGCTACTC | CTGAATAAGCAGAGCCTCACTG | 557 |
| 34 | ACGATGTCATCTGCCCTAGC | TCATGGTCCTGAAAAGCACA | 526 |
| 35 | TCATAGTTACCCAACAATGAAGC | AGTTTCATTGAGATTAGTTTTAAGTGG | 574 |
| 36 | CGCAATATTCTATATGAAAATACCACT | TGAGTGATGGATTTGAACAGAAA | 487 |
| 37 | CCCTTTGTATTTTCTGCATGTG | GGGAGGAGTGGCGTTTATCT | 517 |
| 38 | TGCATGTATGTTCAGCTCTGG | TCAAAAGAAAATTGCTGGGC | 578 |
| 39 | CAGGTGCCCCTAAAAATGTG | GCAACACATCGTTCAAAATCA | 553 |
| 40 | CTTCCTATACATGGGTCCCG | CAAGGAAATGCATCAAATCAAA | 471 |
| 41 | GGGTTATTGAGCGAGGATGA | AAGCCCAAAGTGAGGGAAAC | 506 |
| 42 | GCTTTTAACACTTTCTGGAAAAGTAAG | AGATTTCTGAAGCCAACCACA | 558 |
| 43 | CACCATTTGCTACCTTTGGG | TTCAGCTCATTTGTCTGAATTG | 455 |
| 44 | TTGTGTGTACATGCTAGGTGTG | CCAGGCAAACTCTCTCATCC | 541 |
| 45 | GGGAAATTTTCACATGGAGC | CCTTTAAGCAATCATGGGTGA | 571 |
| 46 | TGAATCAGAATTTTTCTTGTTCGAT | TAAGCGCTAGGGTTACAGGC | — |
| 47 | GAGGGGGTGAGTGTTTCAGT | AAAGCCATTCACCATCATCA | 532 |
| 48 | TCAGTTGCAGTTGGCTATGC | GTGAGGTTGGTTTAGCC | 809 |
| 49 | TCTGTTTCTTTTCTCTGCACCA | GAGTCCTTTAAAGCAATGACTCG | 487 |
| 50 | TATTTGATGGGTGGTTGGCT | CGGTTGTCATGCAACACTTT | 490 |
| 51 | TCATGAATAAGAGTTTGGCTCA | TTAGGCTGAATAGTGAGAGTAATGTG | 522 |
| 52 | CGGAATGTCTCCATTTGAGC | TGCTTTGCAACTATATAAGCCC | 605 |
| 53 | TGTTGTTCATCATCCTAGCCA | AGCCTGGGTGACAGTGAGAC | 507 |
| 54 | TTTGTCCTGAAAGGTGGGTT | AGAAGTCTGAGCCAAGTCCG | 506 |
| 55 | TGTCATTCTTGCATGCCTTC | CCTCCTTGTCCAAATACCGA | 565 |
| 56 | CAATACGCCAAGAAAAGGGA | TGATGTCTTAATATGCATGTCTCC | 589 |
| 57 | CCTCTGTTTTGTGGCTCTCA | GCCAAAAGAGATGGACGATT | 531 |
| 58 | AACACAGCGCTTTCCTCATT | TTCCTCCTCACAGATAACTCCC | 595 |
| 59 | GGGCTGTATCAAAATTTATGCC | TTGTGGGAAGATAACACTGCAC | 514 |
| 60 | ACTGGCACTGCACCCTAAAG | AATTTGAAAATGTTTAGATGGGAA | 410 |
| 61 | ATCCTTTGTGTTTGGCCTTG | ATCCAATTGGCCTTCCTCTT | 475 |
| 62 | CGCATTTATCTTTGTGCCTG | CGCAAAGATTGACTCCCACT | 587 |
| 63 | GGGCCTTTCTGCTTGTAAGA | CAAAGACCTATAGGCCCTCTCA | 489 |
| 64 | GTTGTCAAAGGGCAAAAGGA | AGCTGAGGAATGGTGACAGG | 492 |
| 65 | TGTGGTTCACGTTTGGTGTT | GAGAGCAATCTACATTCTGGCTC | 529 |
| 66 | TGGTTGAATTTCCATTGCAT | TTGACAAGGAATGGCACAAA | 470 |
| 67 | GCACAAATTAGAAGTAACCCCA | CCTGCTGCAGATGGAGATTT | 520 |
| 68 | AGCTGTGAAAAGCCAGCCTA | GGGTAGCTCTTTGGATATCAGG | — |
| 69 | AGCTGAGTTTTTCTTCCCTCC | GAAGCCTACAGTTGAGAGCCA | 500 |
| 70 | TTGAGTAGCCTAGTAAGCTTGTATGT | AAAGTGGCAACTGGACATCAG | 596 |
| 71 | GATCAAAGGGGACGTCTTCA | ATGTCCAGTTGCCACTTTCC | — |
| 72 | CGATGGGAATTTTCCAGAGA | CCGGAAATGTTTAAAAGCCA | 554 |
| 73 | TGGTCTACCACACACTGCCT | AAGATCACGTTTCCACTCCC | 643 |
| 74 | TGGTAGATCACAACCTCAGCA | CTGCAAATGGAGCTAAACAGA | 469 |
| 75 | GCCTCTTTTGCTTGCTGTTC | TCAGTTTGCAGGCACATACC | 522 |
| 76 | GGGAGCACAATTCAGATACAAA | ACAAGTTTTCTGTGGGCCAG | 527 |
| 77 | TGTATGGATTTCTTCTTCCCTTT | GAAACATGTTGCCCTCACG | 482 |
| 78 | GCTGCAAGTGGAGAGGTGAC | GGGACTACAAAGGATTGCCA | — |
| 79a | TTCTTCCTGGAAACTGGTGAA | GCACACTTTAGTTTACAATCTTTCTTT | 599 |
| 79b | AACAATGGCAGGTTTTACACG | AAGCAGGTAAGCCTGGATGA | 581 |
| 79c | GGCAGGCTTGAGTTTTCATT | TACTCCTTCACAGGGATGGG | 584 |
| 79d | ACATTCAGCTTCCTGCTGCT | AACCTGTCTAATCCACCAAGAA | 573 |
| 79e | CAGGTATCAACCCAGAAGCC | GAGCTTTGGGTTTTCTTTTGAA | 600 |
| 79f | TTTGGAGAGTGGGCTGACAT | GGTGGTTATAAAGAACACAACACG | 599 |
| 79g | AAATCAGAGGTAAATAGAGTGCATAAA | GGGGAAGGGGTAGTTAGGAG | 597 |
| Mp1 | TACTCATTGCAGTCGCAAGC | TGATGATGCCAACAGTGTGAA | 581 |
| Mp2 | GCATAATTCACAACTGAAATTTAGGA | GTAGAGGCCCCCGGATATT | 654 |
| Lp1 | AAAACAGAATAAAGCTTCTAGATATGG | GAATCTGCTTTACAGTGGTTGAG | 708 |
| Pp1 | GGTGTCTTCATAATAATCAGCTCC | CTCACAACAAAAGCCCCAA | 658 |
| Cp1 | TCAGCCAAAATTTCAGTGTG | GCAGAGTTTGAAGAGCTCGG | 637 |
| 260p1 | CCAATAAGTTGCCTGCCCTA | TGTGAAGGAGAAAAATAAATAGCAAA | 637 |
| 140p1 | TCAGCAAACCTTGCATTTTT | CACGCTCCTGCATCAGAATA | 674 |
| 116p1 | CAAAGCCTCCATTCATTGT | TGATTTCCCATTTAATACACATTTTT | 610 |

The primer sequences in Table 2 are SEQ ID NOs: 187-372, respectively (internal primer A, internal primer B, from top to bottom).

The sequence reactions were assembled by transfer of a uniform concentration of PCR product to a new cycle sequencing plate along with 10 picomoles of sequencing primers, and the samples with primers were evaporated to dryness in a speed vacuum system. The fragments were rehydrated with a mixture of ABI PRISM BigDye terminators v.3.0, the plates heat-sealed with a foil seal, and placed on thermocycling blocks for cycle sequencing. Post-cycling processing involved ethanol precipitation in the cycling plates, rehydration in formamide and re-sealing. The plate was then placed on the plate deck within the ABI 3700 for robotic loading, capillary electrophoresis, and fluorescent detection of the sequence ladders. All plates within the system were bar code labeled with plain sample identifiers. These bar codes were captured at multiple steps of the process using a web-based system for plate tracking.

1. Sequence Analysis.

After initial data processing using ABI 3700 instruments, sequence trace files were transferred onto a Linux disk server. The base calls were reanalyzed with the Phred program (Ewing et al. (1998) Genome Res 8:175-185) that adds a quantitative base quality value. This base quality value provides a probabilistic estimate of the correctness of the base call. The quality values are the log of the probability that the base call is correct, such that a Phred value of 20 corresponds to a 99% probability that the base call is accurate, while a Phred value of 30 corresponds to a 99.9% probability that the base call is accurate. The sequence was assembled with dystrophin consensus sequence using the Phrap program, and potential mutations were identified using the Consed program. The read assembly was performed on a PCR fragment basis, and a single PCR Phrap assembly consisted of the consensus genomic sequence and all sequence reads relating to the PCR. The read sequence and Phred quality values were compared to the assembled consensus sequence using cross_match, and all discrepancies were tagged and ranked depending on Phred quality of the base (cutoff of 15). All PCR assemblies (Reads+consensus sequence and tagged discrepancies) were then compiled into one consed project for review. Potential base discrepancies were catalogued using Perl scripts, and underwent human review of original trace files. This final list of reviewed discrepancies was loaded into an Oracle database where they were further reviewed in a web browser.

Nucleotide sequence position was based on the annotated mRNA sequence found in GenBank (NM-004006) which encodes the dystrophin Dp427m isoform.

### B. Example 2

### Description of DMD Patient Population Used in SCAIP Sequencing Analysis

Patients from the University of Utah's Muscular Dystrophy Association clinic were ascertained for disease status. The diagnosis of a dystrophinopathy was determined by the presence of clinical features consistent with Duchenne (DMD) or Becker (BMD) muscular dystrophy, along with either (1) absent or altered dystrophin expression by immunohistochemical or immunofluorescent analysis, or immunoblot analysis; or (2) a clear X-linked family history. Some patients had previously had confirmation of dystrophin deletions by clinical testing. Probands from 42 families were enrolled. Forty-two were males with dystrophinopathy by the above criteria; the forty-third was an obligate carrier female (and the mother of two deceased Duchenne patients) with adult onset limb-girdle weakness which led to wheelchair dependence in her sixth decade. Nine additional DNA samples were obtained from self- or physician-referred patients nationwide who had been shown to be deletion-negative on standard screening.

Patients were catalogued as to whether they harbored large-scale dystrophin deletions detectable by standard clinical multiplex PCR analysis. Blood samples for DNA analysis were obtained under an IRB-approved protocol from patients who either had no clinical record of dystrophin deletion testing (unknown deletion status) or who had no detectable deletion by commercial testing. DNA was obtained from each blood sample using a salting-out method (PureGene, Gentra Systems, Inc; Minneapolis).

Direct sequence analysis was also performed on 66 DNA samples from one clinical center (O.S.U.). Sixty-four of the samples had previously been evaluated by the DOVAM-S technique. Clinical phenotype of this set of patients was confirmed by clinical exam and muscle biopsy.

SCAIP detected dystrophin mutations in 70% of patient samples which did not have deletions of more than one exon. Excluding five patients with duplications from the Utah/referral set, the detection increased to 74% (62/84). This is probably an underestimate of the actual rate of detection in the general non-duplication sample population, as duplication testing was not performed on the DOVAM-negative/SCAIP-negative set (n=17).

Correlating these numbers to the general dystrophinopathy population is unhelpful, because the patient set was not a random sample; it likely represented a population enriched in duplications as well as stop codons and subexonic rearrangements. The absence of detectable mutations in the remaining patients is not yet explained, but unlike the case when DOVAM or DHPLC screening is performed, the known coding regions of the dystrophin gene do not contain disease-causing subexonic mutations.

### C. Example 3

### Large Scale (A Exon) Deletions

Deletion status was determined by reviewing clinic records or obtaining clinical (multiplex PCR) testing in 42 Utah probands. Of all the samples, such deletions were found in 25/42 (59.5%) patient samples. As discussed below, a single Utah sample had a non-hotspot single-exon deletion, bringing the total found in the Utah cohort to 26/42 probands, or 62%.

### D. Example 4

### Direct Sequence Analysis by SCAIP Sequencing

1. Amplification Efficiency and Deletion Detection

In anticipation of direct sequence analysis, PCR amplification was performed on 94 samples. These included the remaining 17 Utah probands without multiplex deletions, and 9 referral samples (total unique families n=26); two relatives of Utah probands (1 asymptomatic carrier mother, and 1 affected sibling); and 66 samples from O.S.U. (64 DOVAM-screened and 2 unscreened). PCR amplification was performed on a total of 94 specimens. An aliquot of each well from the 96 well PCR amplification plate was loaded in 96 well format onto an agarose gel. Electrophoretic separation distance for each band was ˜1.8 cm, as the wells were angled slightly relative to the migration path. The products were from a multiexon deletion case missing exons 20 to 30 and the DMD260 promoter. Products corresponding to exons 1 to 78 are located in sequential wells, starting left to right and top to bottom, followed by the multiple exon 79 and alternate promoter products. Note the absence of products in wells corresponding to exons 20 to 30 and Dp260.

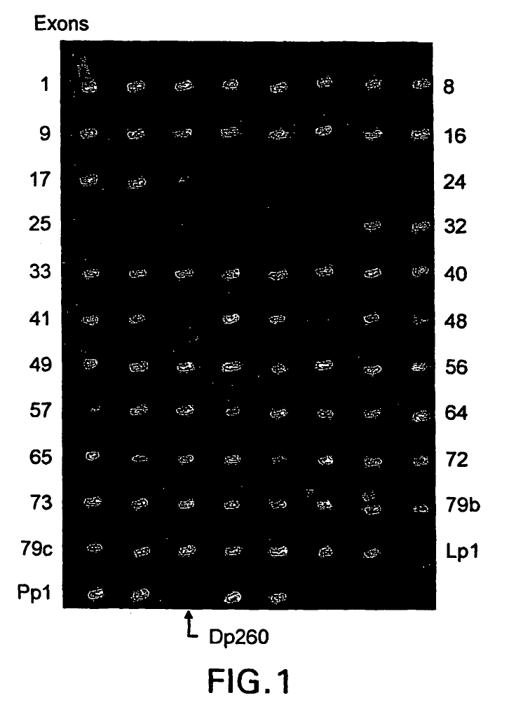

Analysis of PCR products by visualization on agarose gels resulted in the identification of three individuals with deletions of ≧1 exon as shown in FIG. 1. In one OSU case, multiple amplification products from adjacent exons (the DMD260 promoter, and exons 20-30) were missing; review of records (unblinded only after the entire sample set was analyzed) showed that this had been detected by DOVAM analysis. In two patients, single amplification products were not present in exons not screened in commonly-used multiplex screening sets; in each case, PCR was repeated with internal primers in order to exclude the presence of polymorphisms at the primer sites, and the absence of a product on the second round of amplification was interpreted as representing single exon deletions. One Utah patient had a deletion of exon 18. One OSU patient had a deletion of exon 21; unblinded post-amplification review of the DOVAM results showed that a possible deletion had been suspected, but that a primer site polymorphism could not be excluded. The overall efficiency of PCR is summarized in Table 3.

TABLE 3
Efficiency of PCR Recovery.

| | 94 individuals × 93 PCRs = | |
|---|---|---|
| PCR recovery | 8742 PCR potential products | efficiency |
| Primary | 8716/8728 | 99.86% |
| amplification: | Total exons = 8742 − 14 deleted exons = | |
| | 8728 potential products | |
| Primary | 8396/8449 | 99.37% |
| sequencing: | Three deleted samples not sequenced = | |
| | 93 × 3 = 279 exons | |
| | Total exons = 8728 − 279 = 8449 | |

Excluding exons determined to be deleted in these three patients, the efficiency of primary PCR recovery (defined as the presence of a band on first pass, single plate amplification) was 99.86%.

2. Sequencing Efficiency and Quality.

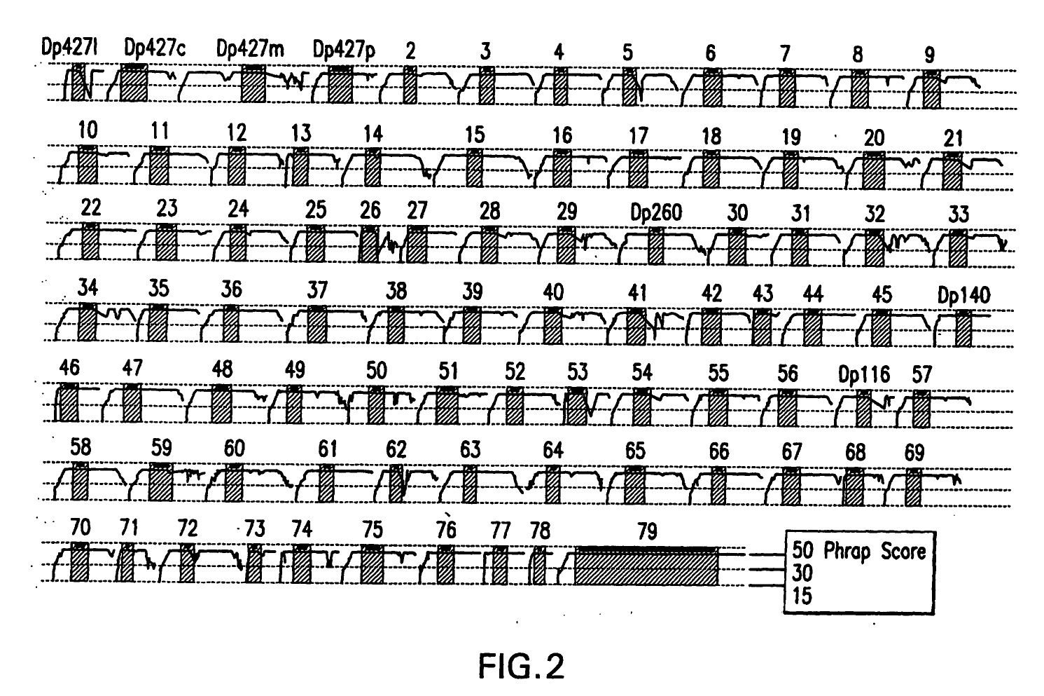

Direct sequence analysis was performed on 91 individual samples. The overall quality of sequence recovery is shown in FIG. 2. Each block represents the length of the individual PCR products, with the exonic sequence indicated by the thick line on the top horizontal axis. The average Phrap score observed in this study is plotted along its horizontal position, with the vertical axis ranging from Phrap score 15 to 50. Phrap scores >50 are not shown, and the portions of the plot corresponding to the exons +/−100 nucleotides are indicated in gray. The Phrap score over coding regions of the gene is generally >60. The efficiency of primary sequencing recovery (defined as high quality sequence on the first sequencing reaction) was 99.37%.

### E. Example 5

### Mutation and Polymorphism Detection

Among the samples from the 16 Utah probands and 9 referral samples, mutations were detected by SCAIP sequence analysis in 16; five additional samples harbored duplications (see below), resulting in an overall detection efficiency of 80% in this group (16/20 non-duplicated patients). The mutations are summarized in Table 4. These include ten stop codon mutations; one single base pair (bp) insertion; and one single bp deletion. The single base pair insertions and deletions were easily detectable as mixed base calls in the two females tested.

In two referral samples, sequence variations were detected that may be causative of disease by altering intronic splice signals. One sequence variation is highly likely to cause disease, as it occurs in the highly conserved +1 position in intron 25 (changing a G to a C). The other is less definitively causative, as it occurs in the less conserved −9 position in intron 11. Both are unique in our series (n=94) and are previously unreported, according to the Leiden database of dystrophin mutations (http://www.dmd.nl/dmd_all.html). Definitive assignment of a causative status to these two will sequence variations will require analysis of dystrophin transcripts; muscle samples are at present unavailable, although further studies are planned.

Of particular interest are two substitutions which result in nonsynonymous changes in amino acid sequence in highly conserved functional domains of the dystrophin protein. One of these, in a boy with a DMD phenotype (loss of ambulation at age 10 years) substitutes a phenylalanine for a cysteine in the dystroglycan binding domain, in a residue conserved in the dystrophin protein through C. elegans. The second, in a boy with a BMD phenotype (still ambulant at age 16 years) substitutes a valine for an asparagine at a similarly conserved residue in the actin-binding domain.

After direct sequence analysis was performed, dystrophin duplication analysis was performed in 13 samples, including the 9/25 Utah or referral samples without detectable mutations, and the four with presumed mutations discussed above (two intronic and two missense). Duplication analysis was performed using the multiplex amplifiable probe hybridization (MAPH) technique (White et al. (2002) Am J Hum Genet 71:365-74). No

TABLE 4

| | | | Age at: | | | | | | |
|---|---|---|---|---|---|---|---|---|---|
| | | | | Loss of | Mutation | | | | |
| | | | | ambulation | | Mutation | | | |
| ID | No. | Type | Presentation | (current age) | Exon | type | nucleotide | amino acid | novel |

Utah Non-deletion, Non-duplication Samples (n = 12 probands).
Mutations (n = 9 probands)

| Stop codons | 42172 | 1 | DMD | 15 | m. | 9 | y | 47 | Stop | 6868A > T | Lys2290X | + |
|---|---|---|---|---|---|---|---|---|---|---|---|---|
| | 42588 | 2 | BMD | 3 | y | n.a. | (10 y) | 31 | Stop | 4250T > A | Leu1417X | − |
| | 42719 | 3 | BMD | 13 | y | n.a. | (19 y) | 31 | Stop | 4240C > T | Gln1414X | + |
| | 42953 | 4 | DMD | 6 | y | 9 | y | 64 | Stop | 9337C > T | Arg3113X | − |
| | 42970 | 5 | BMD | 20 | y | n.a. | (58 y) | 1 | Stop | 9G > A | Trp3X | + |
| Deletions | 42390 | 6 | DMD | 3 | y | n.a. | (4 y) | 30 | 1 bp | 4103delC | frameshift | + |
| | | | | | | | | | deletion | | | |
| | 42389 | 6a | mother of | asympt. | | n.a. | | 30 | 1 bp | 4103delC | frameshift | + |
| | | | indiv. 6 | carrier | | | | | deletion | | | |
| Insertions | 42359 | 7 | Manifesting | 30 | y | n.a. | (58 y) | 8 | 1 bp | 783_784insT | frameshift | + |
| | | | carrier | | | | | | insertion | | | |
| | | | (female) | | | | | | | | | |
| Missense | 42458 | 8 | DMD | 5 | y | 11 | y | 68 | missense | 9938G > T | Cys3313Phe | + |
| | 42515 | 9 | BMD | 6 | y | n.a. | (16 y) | 6 | missense | 494A > T | Asp165Val | + |
| No mutations | 40818 | 10 | DMD | 7 | y | 10 | y | | n.d. | | None | |
| (n = 3 probands) | | | | | | | | | | | | |
| | 42273 | 11 | BMD | 8 | y | n.a. | (18 y) | | n.d. | | None | |
| | 42965 | 12 | BMD | 13 | y | n.a. | (21 y) | | n.d. | | none | |

Referral Samples (n = 8 probands)

| | | | | | | | | | | | | |
|---|---|---|---|---|---|---|---|---|---|---|---|---|
| Mutations | 42962 | 13 | DMD | 4 | y | n.a. | (5 y) | 53 | Stop | 7720C > T | Gln2574X | + |
| (n = 7 probands) | | | | | | | | | | | | |
| | 42964 | 14 | DMD | 4 | y | n.a. | (7 y) | 34 | Stop | 4693C > T | Gln1565X | + |
| | 42968 | 15 | IMD | 2.5 | y | n.a. | (13 y) | 58 | Stop | 8608C > T | Arg2870X | + |
| | 42969 | 16 | BMD | 3 | y | n.a. | (11 y) | 5 | Stop | 355C > T | Gln119X | + |
| | 42971 | 17 | BMD | 5 | y | n.a | (21 y) | | splice | | IVS25 + 1G > C | + |
| | 42974 | 18 | DMD | 4 | y | 12 | y | | splice | | IVS11 − 9G > A | + |
| | 42986 | 19 | DMD | 2.5 | y | 10 | y | 34 | Stop | 4693C > T | Gln1565X | + |
| No mutation | 42963 | 20 | BMD | 5 | y | n.a | (11 y) | | n.d. | | | |
| (n = 1 proband) | | | | | | | | | | | | |

duplications were detected in the samples with the four presumed mutations. Of the remaining nine samples, duplications were found in five (data not shown). Of the four remaining patients without detected mutations, one patient (#42965) was reported to have dystrophin of an increased molecular weight on commercially-obtained immunoblot analysis, raising the possibility that a duplication remains undetected by the MAPH technique.

### F. Example 6

### Comparison of Assay Sensitivity between SCAIP and DOVAM

The SCAIP method was used to study 66 samples from a second center in a blinded fashion. Sixty-four of the samples had previously been studied by DOVAM, which identified subexonic mutations in 44 of the samples, and possible exonic deletions in two (discussed above). SCAIP analysis detected all 44 mutations as well as a previously undetected stop codon mutation (Glu2035X in exon 42, GAG::2035::TAG) in 1 of the 20 other non-deleted samples. This position is 2 nucleotides 5′ of a common variant GAT::2035::GAG (Asp::Glu) that may have interfered with the SSCP analysis used in the DOVAM test.

TABLE 5
Summary of mutation detection in non-deleted,
non-duplicated probands.

| | # mutations | | |
|---|---|---|---|
| | detected | # samples | |
| Utah samples/referrals | 16 | 20 | 80% |
| DOVAM positive samples | 44 | 44 | 100% |
| DOVAM negative samples | 1 | 18 | 5% |
| DOVAM unscreened samples | 0 | 2 | 0% |
| Total: | 62 | 84 | 74% |

### G. Example 7

### Phenotype/Genotype Correlations

The rapid and economical detection of stop codons and small rearrangements will facilitate the study of sequence context effects on disease expression. However, in the present study, only limited correlations between phenotype and genotype are to be drawn, although the results raise several interesting examples. One patient with BMD, the mildest affected patient in the Utah group, who is still walking at age 58 years, has a mutation resulting in a premature stop signal in the third amino acid of the muscle isoform; the next methionine is at position 124. Another intriguing result is the presence in the relatively small sample size of two stop codon mutations in exon 31, both resulting in the BMD phenotype. Although stop codon mutations are expected to be essentially randomly distributed across the gene (unlike the hotspots found for exonic deletions) (Roberts et al. (1994) Hum Mutat 4: 1-11.), the presence of two exon 31 stop codon mutations raises the possibility that stop codons in certain exons may predispose to a milder phenotype, perhaps due to the influence of such mutations in promoting exon skipping as seen in the mdx mouse (Wilton et al. (1997) Muscle Nerve 20:728-734; Lu et al. (2000) J Cell Biol 148:985-996). The mRNA and protein sequences in these and other patients have yet to be determined.

Two patients had a previously undescribed Gln1565X mutation. These patients are not known to be related, and analysis of single nucleotide polymorphisms (SNPs) reveals different haplotypes over at least a portion of the dystrophin gene, supporting the idea that they are unrelated, although distant relatedness with intragenic recombination cannot be excluded. This example illustrates one of the additional advantages of SCAIP analysis. That is, SNPs are found throughout the gene; some are quite common, others less so. Compared to screening strategies such as SSCP or DHPLC, SCAIP analysis allows one to detect a sequence variation with a greater degree of certainty, and the frequency of such variations can be readily established by comparison to the large and growing database of specific polymorphisms. By cataloging the SNPs throughout the coding and control regions for the dystrophin gene and establishing a rigorous and standardized phenotyping process, one is now enabled to generate testable hypotheses regarding the role of such SNPs on the presentation or progression of disease. For example, polymorphisms in the primary cardiac or brain isoform promoters could conceivably alter the clinical expression of cardiomyopathy or cognitive dysfunction. Studies to address these possibilities are underway.

### H. Example 8

### Implications for Clinical Use Including Genetic Counseling

Application of the SCAIP method to the study and clinical care of dystrophin-related diseases will obviate the need for muscle biopsy in a large number of patients. It will routinely allow rapid detection in an economical fashion of the following gene variations in dystrophinopathy patients: (1) all deletions of >1 exon; (2) small rearrangements of <1 exon in size (deletions and insertions); (3) premature stop codon mutations; (4) splice signal site mutations; and (5) missense mutations. Reports of non-synonymous polymorphisms as disease-causing missense mutations in the dystrophinopathies are rare. Analysis of data generated by the present method will allow identification of variants at highly conserved amino acids in patients without any other sequence variation, leading to identification of greater numbers of missense mutations.

The availability of rapid direct sequence analysis will have an immediate impact upon genetic counseling in the dystrophinopathies. Because approximately one-third of all dystrophinopathy patients harbor de novo mutations, X-linked family histories are often absent, and testing of both known and presumptive carriers can, at present, only be performed with high reliability if a proband's specific mutation is known. In the absence of large-scale deletions, carrier testing relies on haplotype analysis. The high quality sequence acquisition method described herein allows ready identification of point mutations or small-scale rearrangements in the heterozygous state, and will lead to improved genetic counseling for dystrophinopathies as well as for other diseases to which it is applied.

### I. Example 9

### LMGD2A and LMGD2B Detection

Limb-girdle muscular dystrophy type 2A (LGMD2A) is an autosomal recessive disorder caused by mutations in the CAPN3 gene, which encodes the skeletal muscle-specific calpain (calcium-activated neutral protease) (Richard et al., Mutations in the proteolytic enzyme calpain 3 cause limb-girdle muscular dystrophy type 2A. Cell. 1995;81:27-40). Mutations are found throughout the CAPN3 gene and include nonsense, splice-site, deletions/insertions, and missense mutations (Richard et al., Calpainopathy-a survey of mutations and polymorphisms. Am J Hum Genet. 1999;64:1524-1540). There is some evidence for founder effects, however most mutations observed are “private” within affected families. LGMD2B is caused by mutations in DYSF, encoding dysferlin, a skeletal muscle protein associated with the sarcolemma (Bashir et al., A gene related to Caenorhabditis elegans spermatogenesis factor fer-1 is mutated in limb-girdle muscular dystrophy type 2B. Nat Genet. 1998;20:37-42). PCR and sequencing primer systems for SCAIP analysis were developed for both the CAPN3 and DYSF genes. The PCR primers are shown in Table 6 and the sequencing primers in Table 7.

TABLE 6

Primer Pairs Used to Amplify the CAPN3
and DYSF Exons and Promoters.

| | | |
|---|---|---|
| GENE_EXON | FORWARD | REVERSE |

| | | |
|---|---|---|
| CAPN3_1 | GCAGTTCTCAGCTTCTTTCCA | GCTCTGTCATGTGCCCACTA |
| CAPN3_2 | CTGCCCTAACTCTCAAGTTGC | ATTGGTTTGAAGGTCCCAGA |
| CAPN3_3 | TTCCAAGGAAAGACTGGCTG | ACCAGCTCTATGCCAAGGTG |
| CAPN3_4 | TCAATGAGGGAGAAAGTGCC | GTTGAGGAAGGGCTGCATTA |
| CAPN3_5 | GCATTGCAAGTCTTGGATCA | TCAATATACTGAGCAGCCCTC |
| CAPN3_6 | AGCTCCAAGTGTCAGGAAGC | TCAGTATTCTCCAGTGAGCAGG |
| CAPN3_7 | CTCCTTAGGCACGGTCATGT | CACGAGAGAACAGGAAGCTCA |
| CAPN3_8 | GCTTCCTGTTCTCTCGTGTTC | CTTCCACTCCTGGCCCTT |
| CAPN3_9 | CCTGGTCTCAGGAATCTCCA | GAGAGAGGGTGAGGTTGACG |
| CAPN3_10 | TCAGAAGTGACAGCGTTTGC | TCCTTCCCTACATCACCCAA |
| CAPN3_11 | TGGCACTTGGTGATATGATAAGA | GTGCGAGGGAGAAAGTGC |
| CAPN3_12 | AGAGAAATGCCTGAATCGTG | AGAAGACCCGGAGGATGAAT |
| CAPN_13 | TTGTGGGCAGGACTGTGATA | GTGTCACCAGAAGCAAGCAG |
| CAPN3_14 | CTGAGCCACTGGCCACATTA | GACTTTGGGCTTCTCACTGC |
| CAPN3_15 | AGGTCAGTTTGAGAGAGCCAT | TGTGGGTCTGGACAACACAG |
| CAPN3_16 | TATCCTTGTCACTTGCACGA | AAGCTGGTTCTGTCTCAGCC |
| CAPN3_17 | GGCGTTGAGCTTTCACAAT | CTCCTTAAGTTTCCCTGGGC |
| CAPN3_18 | GGCTGGAGAGGTGTGAAGAG | GCTTTCCAGAGCCATCTGTC |
| CAPN3_19 | GGCAGCTCTGATCAGGAAAG | TTGACTGCATTTCGCATCTC |
| CAPN3_20 | TGAACCATGACCCTCCTCTC | GATGTGCAGGCAGAGAATCA |
| CAPN3_21 | GACCTGAAGACACACGGGTT | CGCACTCCGCCTCTACTACT |
| CAPN3_22 | CCTGGGTTACAGAGTAGGCG | GCAGCCACTGAAAGAAGTCC |
| CAPN3_23 | GAGATGCGAAATGCAGTCAA | TCTGCAGACAGCCTAGAGCA |
| CAPN3_24 | ATGGCAAAGGGAGGGTTACT | CCCGTTGTACATGACCCATT |
| CAPN3_EP1 | CAGCGAACACTGGATTCTGA | TGGCTCTCTCAAACTGACCTAA |
| CAPN3_DP1 | TTGTGGGCAGGACTGTGATA | GTGTCACCAGAAGCAAGCAG |
| DYSF_1 | GCTGCCAAATACCCAAATGT | TCTGAGAGAGAGCAAAGGGC |
| DYSF_2 | TTCTGGAGATGGATGTTGTTC | TCCCAACTCAGTTTCAACCC |
| DYSF_3 | GGTGCTCAGGGACTCTCTTG | GCAGGTTGGGTTGAACTTGT |
| DYSF_4 | TGTCAGTCAGAAATGCAGCC | AGGGCGGAAGTAGTTCCAAT |
| DYSF_5 | TGTCACCAGTCCCTCTCCTC | CTGAGACAGGCACAGCACTT |
| DYSF_6 | ATGGAGGTGCAGTAGGTTGG | GCTTGAACAAATTCAAATTCCA |
| DYSF_7 | TCATCCATCTTCCCATTGCT | GCGTGTGCACTGACACCTAT |
| DYSF_8 | GAAGCCAGTGGTGAGATGGT | CATTCACAGGGAACATGTGG |
| DYSF_9 | TAAACTGCTAGGCGTGGAGG | TGGATCATTGCCTGTGATGT |
| DYSF_10 | TTCTGAGAACCCAAGGGTTAAG | CAGCAGCCAGTTCCTGAGAT |
| DYSF_11 | TACAGAGAGCCCCGTGAGTT | AGCCATCAGCCATATTCAGG |
| DYSF_12 | CATCAATGCATGTGGGATGT | GTCTAGTATCGGGCCAACCA |
| DYSF_13 | TGTGTTGAATTCCCTGCAAC | GGTTCGGAGAGCTACGGAGT |
| DYSF_14 | TTGGATCTGGTTTCCACTCC | CTTTCTAAGACGCCCGTGAG |
| DYSF_15 | GAAAGCTGGTCTGGACTGGA | CAACTAGCAGGAGGTGGCAT |
| DYSF_16 | TCTGCATAGGATGTGGTTGG | GAAAGGTCTCGGAGTGCTAA |
| DYSF_17 | TTGTGGACAGTGTCTGGCTC | AGGTCATGCACTGTGAGTCG |
| DYSF_18 | TTAGGGCAGAGGGTATGTGC | ATGACACCTCAAGGCCAGTC |
| DYSF_19 | TGGATGACTACCTGGGCTTC | GGCAGGAACTCAATCCTACG |
| DYSF_20 | CGTAGGATTGAGTTCCTGCC | AGTAGTGGCACCCTGGAATG |
| DYSF_21 | CTGTTTGCGGCCTTCTACTC | TCTCCTTGCACTGGACACAG |
| DYSF_22 | GACAGTCCTTGGCCTCTCAG | TTAACCCTGTGGAGAGCAGA |
| DYSF_23 | TTCTGGGAAGGGTTCTGTTG | GAGCAGACGCTTCTCATTCC |
| DYSF_24 | AGCTGGGAGCAGTTGTCAAT | GCAGCTTTGGCTCTATGTCC |
| DYSF_25 | TTCATGTTGGGTTGTTGTGG | CAGTCCTGGGAGAGTTCAGC |
| DYSF_26 | AATCACTTGAAAGGGTAGGGA | CAGTCCTGGGAGAGTTCAGC |
| DYSF_27 | TCCTCAAAGACACCCAGGAC | ATTTGGCTGAGATCCCTCCT |
| DYSF_28 | TTGGTTGGCATTCAACTGTG | CAGGTCTGCATCTGTGCCTA |
| DYSF_29 | CTCCAGGAGGTGGTAGATGG | GATCTGTGGGTGTTCCCAGT |
| DYSF_30 | GCTGTGGTTGGGAAATAGGA | CTGGATTTCAGAGGGAGCAG |
| DYSF_31 | AAGTGGTCCAGTCTTGGTGC | CGAAAGCCAGATGTCTCCAT |
| DYSF_32 | ATCTGCCATAACCAGCTTCG | AGGGACTTGTCTGCTGTGCT |
| DYSF_33 | CTCACAGACACCAGCAGCTC | CAGCCCATAGCACTCTCTCC |
| DYSF_34 | GAGGAAGAGTCCATGTGGGA | CCATGGTTTGCAGCCTCTAT |
| DYSF_35 | GTTTATGGGTCGCTGCATCT | GCAGCTGAACTTGGCATGTA |
| DYSF_36 | GCACTGGATGCATTACCTGA | GGGCTCTCCTTCCTGTCTCT |
| DYSF_37 | CTTTCTGGCTCACAATGCAA | CAGACCTGCCTTACTCTGGC |
| DYSF_38 | CTTTCTGGCTGACAATGCAA | GCTTCTGTTGACAGCCACTG |
| DYSF_39 | GCCTAGACCTAGTGGCCAGA | GGGCTCCTTGTCATCAATGT |
| DYSE_40 | GGAGAGCTTCCTGTGTGACC | AGGGTGACAACCTGGAACAG |
| DYSF_41 | AGGTCAGGATTTGCCACAAC | CACAGAAACAGGGTTTCCCA |
| DYSF_42 | AACCTGTGTCACTTGCATAATTAAA | GGGTCACCAGTGTAGGTACGA |
| DYSF_43 | GAAGACATACCCAAGACTTGG | ACCTGGGACTCTGCCATGA |
| DYSF_44 | CTTGAAGCCTTCCTGATGCT | CCTCTAGCTCTTGCTACAAACACA |
| DYSF_45 | AATTCTCCCTCCATCCCATC | GTCCAGAGCTGAGGAGCAAG |
| DYSF_46 | ACAGGCTGCTGTCCAAGTTT | GCATCTCAGACACACGGAGA |
| DYSF_47 | CCTAGCAGGGAGGAGCTGTA | GCATCCTCATGGCTCACTTT |
| DYSF_48 | AAAGTGAGCCATGAGGATGC | TCTTCAAAGCCAATCATCCA |
| DYSF_49 | CTGAACGGTGCTCTTTGACA | CTTTAGAAGCCCTGGTGCTG |
| DYSF_50 | TCTTAAGGCCTTCCCATCCT | AAGCAACTCCCAATCCTGTG |
| DYSF_51 | TTTCAGCAGGAGACGGAACT | CTGCTCTCACAGATGAGCGT |
| DYSF_52 | TAATTGAAGAGGTGGGTGGC | TGCTTTGCAGACATTGGTAAT |
| DYSF_53 | GAAATGCTCATTGCTGCTGA | TCCAGCAAACACATTCCTGA |
| DYSF_54 | GAGACCCGTGAGACACCAGT | CCAAGTGAAAGGAAACCCAA |
| DYSF_55 | GCTCTGTTTCCAGAGTTGGC | AATAGGCCAAAGCCAGAGGT |

The primer sequences in Table 6 are SEQ ID NOs: 373-534, respectively (forward primer, reverse primer, from top to bottom).

TABLE 7

Primer Pairs Used to Sequence the CAPN3
and DYSF Exons and Promoters.

| | | |
|---|---|---|
| Gene_Exon | Internal Primer A | Internal Primer B |

| | | |
|---|---|---|
| CAPN3_1 | TCTCAGATGACAGAATTACTCCAA | CAGAGCTGCTGCCAGGAT |
| CAPN3_2 | CTGGCCAACATGGTGAAAC | GATGCATGGCAGAGTGCTAA |
| CAPN3_3 | CCTGTTGATCATATTGTCAAGGAA | AGGGATTAGGGAGCCAGAGA |
| CAPN3_4 | GCACCCAGTCCAGTTAGAGA | TTAGAGCTGTTGTTGCCTGG |
| CAPN3_5 | TCTTGGGTGGGTCACTTAGC | TCCCTTGAGAAATTCCCAGTC |
| CAPN3_6 | ATGGACAGCTTGGAAGGTCA | CTGGTTCTTGCACCCTCTTC |
| CAPN3_7 | TGGTCAGGACAGAGCCTTCT | AAACTGTGCACCAACTGTGG |
| CAPN3_8 | AGATGGCCAAGCCCTAAGTT | CTTCCAGTCCTGGCCCTT |
| CAPN3_9 | TCACCAGCCCATTTAAGGAG | CTGGAATAGAGTGTGTGGCG |
| CAPN3_10 | TCAGAAGTGACAGCGTTTGC | CAAGCAGCATCTGCATTGTT |
| GAPN3_11 | CTCCATCTGAATAAAGGTAGCG | CGCTCCACTGCCTCTCTAAT |
| CAPN3_12 | ATACTTTCCCAGGGAGGACG | GAGTGTGCAAAGGCATGTGT |
| CAPN3_13 | ATTTAAGCCTTGGGAGTCGG | GCCTGGAACATAGTAGGTGCTC |
| CAPN3_14 | CTCTGTCGTTGGAAGATGCAC | GACCCTCTTCCATATTTCCCA |
| CAPN3_15 | CCTTGCCATATGCAGTAAGAG | TAGGGCTGTTGTGAGGAAGG |
| CAPN3_16 | AGGAGGGATGGAGTGGGTAT | CCTGCCAGTCCACTCCTAGA |
| CAPN3_17 | CGCCATATCTCCTTTGGCT | GCACCTCAGCTATCAGGACC |
| CAPN3_18 | CACACAAATCCACAAGCCCT | CACCCTGTATGTTGCCTTGG |
| CAPN3_19 | AACACAGCCAGGTGGAATTT | CAGGCCTGAGAGAAGCACA |
| CAPN3_20 | TGTTGGGTTGTAACTGCCCT | ATTCCTGCTCCCACCGTCT |
| CAPN3_21 | TAGACCCTCCCTCCAAATCC | GCTGGTTGTTGAGGTGGAAT |
| CAPN3_22 | GAGATGCGAAATGCAGTCAA | AGCACAAAGATGTGCAGGC |
| CAPN3_23 | TGATAATCTCCAGTCTGCTCCA | GCAGTGGCTTACTGTTTCCTTT |
| CAPN3_24 | CAGGACACATGCACTTGAGG | ACTTTCCTCCACATGGCAAA |
| CAPN3_Ep1 | ACAGAGTGCTGTGTGTTGGG | GACACTGGAGCGAAATGTCA |
| CAPN3_Dp1 | TTGCATGACCCATGACTACC | CTTCCCAACTCCCTGGTCAC |
| DYSF_1 | GAGCCTTTCTCCTGTCCAAG | CTAGGTGCTCTCCAGGGTTG |
| DYSF_2 | TTAAGGAGAGTCAGCCTGGG | CAAGAGAGTCCCTGAGCACC |
| DYSF_3 | GGGTTGAAACTGAGTTGGGA | GGAAGCTCAGCTGTACCCAT |
| DYSF_4 | TTCCCATGCCCAAGTATTTC | CCTCTGCCCTTCCCATCT |
| DYSF_5 | GCCTAAGGTCACACAGCTCC | CACATTACTCCCTGCACCG |
| DYSF_6 | GACTGCCCTCAAGTTTCAGC | AACTCCCTGTTTGGCATCTG |
| DYSF_7 | CAGCCTGGCAGCTCTTCTAT | ATAGGGTGACAGGGATGTGG |
| DYSF_8 | TCTGTGGGACTGGAGAAAGG | TTCTGTGACCCGTAGAGCCT |
| DYSF_9 | TATGCCGTGTAGGGATTGTG | AGAGGGCTTGGCGTTGTTC |
| DYSF_10 | CTCCCAAAGTGCTGGGATTA | GCTTGTCACCCAAATGACCT |
| DYSF_11 | CAGCCTCTTACAGGCGTTTC | CAGAGGGATGTGCAATGAGA |
| DYSF_12 | ACTGGAGATGTTCCTCGCAC | AGGACATTGGAATGGAGCTG |
| DYSF_13 | AGCTGTTTGGGACTGGTGAC | CAGACCTGTCCACATTCGTG |
| DYSF_14 | GTAGAAGGGCTGTGGCATTC | CGCCCTAAAGACTCCAAGAC |
| DYSF_15 | CCCTGTGTCTTCTAGCTGTGC | CTGCCCTCAGAGATGATTCC |
| DYSF_16 | GCGTCTGTAGAGATCCAGGC | GGCATATCCCACAATCCAAG |
| DYSF_17 | CGGAACACACAGAGTGATGG | TCTAACTCGAGCATCAGCCC |
| DYSF_18 | TTCTTTGCATCTCCAAGCCT | CATGGAAGGATCAGACTGGC |
| DYSF_19 | CATCTGGGTGGCTTGTCATA | GAAGCAGGGCAAGTGTTGAT |
| DYSF_20 | ATGCTGTTTCTTTCTTGGGC | AATGATCAGGATGGGTCAGG |
| DYSF_21 | CACTAGGGAACACGGGTACG | TCTGTGTCCCACTGCACACT |
| DYSF_22 | AGACTGGATGTATTTGGGCG | GCTGCTGCAGGGAGATTTAT |
| DYSF_23 | AGATGGCTGTGTGTGTGGAG | TTCCTTCTGCAAATTGGTCC |
| DYSF_24 | GCCACTCAAGCCAGACACT | TGATTCCGGCTCAAACCTAC |
| DYSF_25 | GGAATGATGTAGCCTTTGCC | TTGGGTAGCTTGATCTTGCC |
| DYSF_26 | GATACGGGTCAAGCTGTGGT | CAGTCCTGGGAGAGTTCAGC |
| DYSF_27 | TCTCGGAGTGTCCCTAGGTC | GGCAAGCAATGAGAGGAGAC |
| DYSF_28 | TACCTCCGGAGACTTCATGC | CTCCTGGGACCATCTCTGAA |
| DYSF_29 | CCCTTCACTGGGCTATTTCA | ATCTTTGGGTATGCTGGGTG |
| DYSF_30 | TTCCTGTGGCTGCAGAAAG | AGCAAGTGTTTCAGTGCCAA |
| DYSF_31 | TTCCGTTCTGACTCATCTGG | GGGCCTTAAATGCCTGATCT |
| DYSF_32 | TGTGGCTGTCCCATTGTCTA | TCAGCGAAGCCTGATCCTAC |
| DYSF_33 | AGGACCCAGGCTCCATGT | GCATCTGTGCTAGCAATCCA |
| DYSF_34 | GTCACCACAGGCTGCTCAC | AACCACGTCAGGAGATGACC |
| DYSF_35 | TGGGTTGGACCTGTACCTTC | TCCTTCCATCTGGGATTCTG |
| DYSF_36 | GCACTGACATCCATCACACC | TTGTCTGGGTGAAATGTGGC |
| DYSF_37 | GGTGCTGGAATTGTGATCCT | GCAGATGTCAAAGTTGGGGT |
| DYSF_38 | GAGGGAGGCCAACATCTACA | CTGAACCCTTCCAGTGAGGA |
| DYSF_39 | TGAACAGGATGCATTTGGAA | CCTAAGGAAGGTCTCCACCC |
| DYSF_40 | AGAGAGGGCAGGGAGACAAT | GGATTGAGTCTTGCCCAGAT |
| DYSF_41 | CCAACCAAATGCTGAAACCT | GTTATCCCAGCCCACACTTG |
| DYSF_42 | GTTCCTTTCTGGCTCCCTCT | AACACCATCCCATCACCAGT |
| DYSF_43 | CACGAGAATAGCATGGGAAA | TACTGACACTGGCCTTCCCT |
| DYSF_44 | TGTTTCTGATAAGGGCCTGG | GGAGCTTCTGTTGGGATCAA |
| DYSF_45 | ACACTCAGGCCCAGTACAGC | TGTGATGAGCCAGGTTCTTG |
| DYSF_46 | TGAGCCTCCATTTCTCCATC | CAGTGGCATCACAGGTCAGT |
| DYSF_47 | AAGCCTGGAGCTAGTGGACA | CAGAGGAAGCCAGGACCTAA |
| DYSF_48 | ATCTCTGAGAAGCCCACCCT | GAAGCCAAGAAGCAGACTGG |
| DYSF_49 | AGAGCCAGAAGGTGACTTGC | CAACCCAAAGTTCAGTGCAG |
| DYSF_50 | TGCACTGAACTTTGGGTTGA | AGACAGCAGTGGTGGTGACA |
| DYSF_51 | TTGGGAGGATTAATGGAGCC | ACCTCTACTGACAGGCCCAC |
| DYSF_52 | GATGGAATGGGAGACAATGG | GGGAGGAAAGAGGGAGAATG |
| DYSF_53 | GCTATGATGCATGCAAATGTT | CTGCATCTTGAATTCGCTGA |
| DYSF_54 | CAGCACCCAGAAGAGGAGG | GGACTAAGAGCCTCCAAGGG |
| DYSF_55 | GTCCTCTCCCAGCCTCTG | ACTGCTTCTCAGCTGCCTCT |

The primer sequences in Table 7 are SEQ ID NOs: 535-696, respectively (internal primer A, internal primer B, from top to bottom).

Program Listing

The following is a program listing of an example of a Perl script for the analysis of primers for use in the disclosed method.

#!/usr/local/bin/perl
#####################################
#### Primer Prediction Utility
#####################################
use Getopt::Std;
use Bio::Seq;
use Bio::SeqIO;
use Bio::SeqI;
use Bio::SeqFeatureI;
use Bio::Tools::CodonTable;
use Getopt::Std;
use Cwd;
use Getopt::Std;
use Storable qw{dclone retrieve store};
#### Get Parameters
getopt(′o::l::L::s::p′);
### Error out if the required parameters are not passed
if (!$opt_o ∥ !$opt_s ∥ !$opt_l ∥ !$opt_L) {

die “Usage: single_primers.pl -o SEQOBJ.store -l Smallest -L Largest -s GenomicFlank

to grab
(-p 1 * for PCR primers, leave off if for sequencing primers)\n\n”;
}
#### Get Bio::Seq Object
eval{ $in=Bio::SeqIO−>new(′-file′ => “$filename”,

′-format′ => ′GenBank′);

};

$seqobj = retrieve “$opt_o”;
#### Retrieve Exons for the Seqobj
(@exons) = &feature_array(“exon”);
if($exons[0] == −1) {

die “No Exons in $opt_o\n”;

}
$exon_number = scalar(@exons);
#### Make a genomic file
&make_genomic;
print “There are $exon_number exons\n”;
#### Process the exon info
$exonc = 0;
print “Processing Exon Info\n”;
foreach (@exons) {

$exonp++;
$start = $_−>start( );
$end = $_−>end( );
print “START $start −> $end\n”;
$size = $end − $start;
$flank = $end;
$flank −= $start;
### calculate the distance for the exon from the end of the sequence segment
### and then extracts the segment of sequence with the exon centered in it
if ($flank < $opt_s) {

$flank = $opt_s − $flank;
$flank /= 2;
$flank = sprintf (“%.0f”, $flank);
$start −= $flank;
$end += $flank;

} else {

$start −= 250; ## for sequence
$end += 250; ## for sequencing
$flank = 250;

}
$exoncoords{“$exonp”} = “$start,$end”;
$flank{“$exonp”} = $flank;
$size{“$exonp”} = $size;

# print “$exonp = $start,$end\n”;
}
#### Now that we have exon info lets get the sequence
(@GENOMIC) = split(//,$seqobj−>seq( ));
### if PCR Primers mask Repeat Elements (Repeats are marked in the seqobject)
if ($opt_p) {

my $temp;
(@Repeats) = &feature_array(“misc_feature”,“note”,“RepeatMask”);
foreach $r (@Repeats) {

$start = $r−>start( );
$end = $r−>end( );
$temp = $start;
while ($temp <= $end) {

$GENOMIC[$temp−1] = “N”;
$temp++;

}

}

}
#### Lowercase all exons
(@e2) = &feature_array(′exon′);
foreach $r (@e2) {

$start = $r−>start( );
$end = $r−>end( );
$temp = $start;
while ($temp <= $end) {

$GENOMIC[$temp−1] =˜ tr/[A-Z]/[a-z]/;
$temp++;

}

}
$total_g = scalar(@GENOMIC);
print “Total bases = $total_g\n”;
#### now that i have the genomic i am going to extract the exon genomic ( minus 100 bases
for the sweet spot of sequencing)
print “Partitioning Exon Sequence\n”;
foreach (sort keys %exoncoords) {

($start, $end) = split(/,/,$exoncoords{“$_”});
$start −= 1; #want 100 bases not 99
$end += 1;
print “Coord = $_$start,$end\n”;

# print “$start, $end\n”;

$glob_start{$_} = $start;
$glob_end{$_} =$end;
$basec = 0;
foreach $agct (@GENOMIC) {

$basec++;
if ($basec == $start) {

$base on = 33;

}
if ($basec == $end) {

$base_on = 87;

}
if ($base_on == 33) {

if ($agct =˜ “G” ∥ $agct =˜ “C”) {

$gc++;

}
$exonsequence{“$_”} .= $agct;

}

}
$gc_content = $gc;
$gc = “”;
####### Mask Sequence Runs
$exonsequence{“$_”} =˜ s/GGGGGG/NNNNNN/g;
$exonsequence{“$_”} =˜ s/GGGGG/NNNNN/g;
$exonsequence{“$_”} =˜ s/GGGG/NNNN/g;
$exonsequence{“$_”} =˜ s/CCCCCC/NNNNNN/g;
$exonsequence{“$_”} =˜ s/CCCCC/NNNNN/g;
$exonsequence{“$_”} =˜ s/CCCC/NNNN/g;
$exonsequence{“$_”} =˜ s/TTTTTT/NNNNNN/g;
$exonsequence{“$_”} =˜ s/TTTTT/NNNNN/g;
$exonsequence{“$_”} =˜ s/TTTT/NNNN/g;
$exonsequence{“$_”} =˜ s/AAAAAA/NNNNNN/g;
$exonsequence{“$_”} =˜ s/AAAAA/NNNNN/g;
$exonsequence{“$_”} =˜ s/AAAA/NNNN/g;

}
### Create directories
if ($opt_p) {

if (!-d “pcr_pr3”) {

‘mkdir pcr_pr3‘;

}
$dir = “pcr_pr3”;
$oli_file = “PCR_OLI”;

} else {

if (!-d “seq_pr3”) {

‘mkdir seq_pr3‘;

}
$dir = “seq_pr3”;
$oli_file = “SEQ_OLI”;

}
#### Generate an error log
open(ERROR, “>$dir/error.log”);
print “Printing Sequnece Info\n”;
open(EXONFASTA, “>$dir/exons_seq_fasta”);
open(DMDOLI, “>$oli_file”);
foreach (sort keys %exoncoords) {

($start, $end) = split(/,/,$exoncoords{“$_”});
$flank = $flank{“$_”};
$target_start = $opt_s − $opt_l;
$target_start /= 2;
$target_start = sprintf(“%.0f”,$target_start);
$target_size = $opt_l;

### Target size is the smallest acceptable product size

$target_size = sprintf(”%.0f”,$target_size);
open(EXONIND, “>$dir/EXON_$_\_FASTA”);
open(PR3TEMP, “>$dir/PR3.tmp”);
print EXONFASTA “>EXON_$_\n”;
print EXONIND “>EXON_$_\n”;
print PR3TEMP “PRIMER_SEQUENCE_ID=EXON_$_\n”;
$exonsequence{“$_”} =˜ tr/[X]/[N]/;

## Some sequence has X's intead of NN's, primer 3
doesn't like X's

print PR3TEMP “SEQUENCE=$exonsequence{$_}\n”;
(@exons) = split(//,$exonsequence{“$_”});
$exon_seq_count = scalar(@exons);
print PR3TEMP “TARGET=$target_start,$opt_l\n”;
print PR3TEMP “PRIMER_NUM_NS_ACCEPTED=0\n”;
print PR3TEMP “PRIMER_PRODUCT_SIZE_RANGE=$opt_l-$opt_L\n”;
print PR3TEMP “PRIMER_EXPLAIN_FLAG=l\n”;
print PR3TEMP “=\n”;
close PR3TEMP;
print “Exon $_has $exon_seq_count in its PCR Region\n”;
$basec = 0;
$nl = 60;
foreach $e (@exons) {

$basec++;
print EXONFASTA “$e”;
print EXONIND “$e”;
if ($basec == $nl) {

print EXONFASTA “\n”;
print EXONIND “\n”;
$nl += 60;

}

}
print “Picking Primers for $_\n”;
@primer3 = ‘primer3 < $dir/PR3.tmp > $dir/EXON_$_\_PR3‘;

### PRIMER3 Prediction program

close EXONIND;
print EXONFASTA “\n”;
### Lets Process the PR3 Output
chomp($left_pcr_pos = ‘grep “PRIMER_LEFT=” $dir/EXON_$_\_PR3‘);
chomp($left_pcr = ‘grep “PRIMER_LEFT_SEQUENCE=” $dir/EXON_$_\_PR3‘);
chomp($left_pcr_tm = ‘grep “PRIMER_LEFT_TM=” $dir/EXON_$_\_PR3‘);
($label, $left_pcr) = split(/=/,$left_pcr);
($labe,$left_pcr_tm) = split(/=/,$left_pcr_tm);
chomp($right_pcr_pos = ‘grep “PRIMER_RIGHT=” $dir/EXON_$_\_PR3‘);
chomp($right_pcr = ‘grep “PRIMER_RIGHT_SEQUENCE=” $dir/EXON_$_\_PR3‘);
chomp($right_pcr_tm = ‘grep “PRIMER_RIGHT_TM=” $dir/EXON_$_\_PR3‘);
($label, $right_pcr) = split(/=/,$right_pcr);
($label,$right_pcr_tm) = split(/=/,$right_pcr_tm);
undef($lglobal_start);
undef($lglobal_end);
undef($rglobal_start);
undef($rglobal_end);
if ($left_pcr_pos =˜ [symbol] d+,\d+/) {

($j,$pos) = split(/=/,$left_pcr_pos);
($st,$len) = split(/,/,$pos);
$lglobal_start = $glob_start{$_} + $st + 1;
$lglobal_end = $lglobal_start + $len;
($j,$pos) = split(/=/,$right_pcr_pos);
($st,$len) = split(/,/,$pos);
$rglobal_start = $glob_start{$_} + $st − 1;
$rglobal_end = $rglobal_start − $len;
open(OLI, “>$dir/EXON_$_\_OLI”);
print OLI “>EXON_$_\_LEFT TM:$left_pcr_tm\n”;
print OLI “$left_pcr\n”;
print OLI “>EXON_$_\_RIGHT TM:$right_pcr_tm\n”;
print OLI “$right_pcr\n”;
close OLI;

}
print DMDOLI “>EXON_$_\_LEFT TM:$left_pcr_tm START:$lglobal_start END:

$lglobal_end\n”;

print DMDOLI “$left_pcr\n”;
print DMDOLI “>EXON_$_\_RIGHT TM:$right_pcr_tm START:$rglobal_start

END:$rglobal_end\n”;

print DMDOLI “$right_pcr\n”;
if (! $left_pcr ∥ ! $right_pcr) {

print ERROR “EXON_$_NO PRIMER\n”;

}

}
close EXONFASTA;
close ERROR;
close DMDOLI;
### Masked Sequence Subroutine
sub make_masked {

(@genomic) = split(//,$seqobj−>seq( ));
(@Repeats) = &feature_array(“misc_feature”,“note”,“RepeatMask”);
foreach $r (@Repeats) {

| | $start = $r−>start( ); |
|---|---|
| | $end = $r−>end( ); |
| # | print “$start −> $end\n”; |
| | $temp = $start; |
| | while ($temp <= $end) { |

$genomic[$temp−1] = “N”;
$temp++;

}

}

# die;

open(MASK,“>$opt_o.masked”);
print MASK “>$opt_o\_masked\n”;
$lb = 50;
$c = 0;
foreach $g (@genomic) {

print MASK “$g”;
$c++;
if ($c == $lb) {

print MASK “\n”;
$c = 0;

}

}
close MASK;

# die;
}
### Genomic Output Subroutine
sub make_genomic {

$seq = $seqobj−>seq( );
$genomic_query = “$opt_o.genomic”;
open(GENOMIC,“>$opt_o.genomic”);
print GENOMIC “>TEMP\n$seq\n”;
close GENOMIC;

}
### Feature retrieval subroutine
sub feature_array {

undef(@returns);
($tag) = $_[0];
($subtag) = $_[1];
($subvalue) = $_[2];
@all = $seqobj−>all_SeqFeatures( );
foreach (@all) {

if($_−>primary_tag =˜ /$tag/) {

if ($subtag && $subvalue) {

eval{

($cvalue) = $_−>each_tag_value(“$subtag”);
if ($cvalue =˜ /$subvalue/) {

push(@returns,$_);

}

};

} else {

push(@returns,$_);

}

}

}
if ($returns[0]) {

return(@returns);

} else {

return(−1);

}

}

We claim:

1. A method for characterizing a nucleic acid region, the method comprising
(a) adding to each of a plurality of reaction chambers a nucleic acid sample and a different set of amplification primers, wherein each set of amplification primers is complementary to a single amplicon of a nucleic acid region of interest;
(b) performing amplification reactions for each reaction chamber under the same reaction conditions;
(c) bringing into contact in each of a plurality of reaction chambers an amplicon from a different one of the amplification reactions and one or more internal sequencing primers corresponding to the amplicon;
(d) performing sequencing reactions for each reaction chamber under the same reaction conditions; and
(e) analyzing the sequences of the amplicons.

2. The method of claim 1, wherein the nucleic acid region of interest is a multi-exon gene.

3. The method of claim 2, wherein the multi-exon gene is dystrophin, SOD-1 NF-1, ATM, dysferlin, calpain, sarcoglycans, collagen VI, Nebulin, or Titin.

4. The method of claim 2, wherein the amplicons collectively comprise sequence from every exon of the multi-exon gene.

5. The method of claim 4, wherein the amplicons each comprise an exonic region or proximal promoter segment of the multi-exon gene.

6. The method of claim 1, wherein at least 30 amplicons of the nucleic acid region of interest are amplified.

7. The method of claim 1, wherein a single solid support comprises all of the reaction chambers.

8. The method of claim 7, wherein the solid support is a 96 well plate.

9. The method of claim 1, wherein the amplification reactions are PCR reactions and wherein the sequencing reactions are cycle sequencing reactions.

10. The method of claim 1, wherein the amplicons produced in the amplification reactions are purified prior to step (c) and wherein the sequencing products produced in the sequencing reactions are purified prior to step (e).

11. The method of claim 1, wherein the sequences of the amplicons are analyzed by electrophoretic separation and fluorescent detection of nucleotides on a sequence analyzer.

12. The method of claim 11, wherein the sequences of the amplicons are further analyzed by identifying mutations in the nucleic acid region of interest.

13. The method of claim 12, wherein the mutations are deletions, point mutations, frameshifts, or combinations thereof.

14. The method of claim 1, wherein the sets of amplification primers are selected from the group of primer sets as shown in Table 1 or Table 6.

15. The method of claim 1, wherein the sets of sequencing primers are selected from the group of primer sets as shown in Table 2 or Table 7.

16. The method of claim 1, wherein the nucleic acid sample was derived from a patient, wherein the analysis of the sequences of the amplicons indicates dystrophinopathy in the patient.

17. The method of claim 16, wherein the dystrophinopathy is Duchenne Muscular Dystrophy (DMD) and Becker Muscular Dystrophy (BMD).

18. The method of claim 1, wherein the sequences of the amplicons are analyzed by comparing the sequences of the amplicons to other known nucleotide sequences.

19. A primer set which recognizes a single exon or a proximal promoter for the dystrophin gene, the set comprising the primers as shown in Table 1 or Table 6.

20. A primer set which recognizes a single exon or a proximal promoter for the dystrophin gene, the set comprising the primers as shown in Table 2 or Table 7.
